(19) United States
(12) Patent Application Publication
(10) Pub. No.: US 2025/0332243 A1
(43) Pub. Date: Oct. 30, 2025

(54) METHODS OF MANUFACTURING PORCINE ENDOGENOUS RETROVIRUS (PERV) FREE ANIMAL HEALTH VACCINES

(71) Applicant: Zoetis Services LLC, Parsippany, NJ (US)

(72) Inventors: Jacqueline Gayle MARX, Portage, MI (US); Megan Marie Hardesty, Kalamazoo, MI (US); Everett Lee ROSEY, Schoolcraft, MI (US); Robert Gerard ANKENBAUER, Portage, MI (US)

(21) Appl. No.: 19/191,355

(22) Filed: Apr. 28, 2025

Related U.S. Application Data
(60) Provisional application No. 63/640,392, filed on Apr. 30, 2024.

(51) Int. Cl. A61K 39/12; A61K 39/00; C12N 5/071

(52) U.S. Cl. CPC A61K 39/12; C12N 5/0686; A61K 2039/552; C12N 2750/10034; C12N 2750/10051

(57) ABSTRACT

The invention provides a method of preparing a vaccine composition. The method includes infecting gene-edited porcine endogenous retrovirus (PERV) negative swine cells with a microorganism which expresses at least one protein antigen capable of inducing protective immunity in an animal against an infectious agent; culturing the infected cells in culture medium to propagate the microorganism; and harvesting the propagated microorganism from the culture medium to obtain a fraction comprising a PERV free antigen for use in immunizing an animal against the infectious agent.

### CROSS-REFERENCE TO RELATED APPLICATION

This application claims the benefit of U.S. Provisional Application No. 63/640,392, filed Apr. 30, 2024, the entire contents of which are incorporated herein by reference in their entirety.

### REFERENCE TO AN ELECTRONIC SEQUENCE LISTING

The contents of the electronic sequence listing (ZP000498.xml; Size: 58,190 bytes; and Date of Creation: Apr. 16, 2025) is herein incorporated by reference in its entirety.

### FIELD OF THE INVENTION

This application is concerned with the area of animal health vaccines, more particularly to methods of manufacturing PERV free modified live vaccines on CRISPR-edited cell lines intended for animals, such as pigs, including neonatal pigs as early as three days of age.

### BACKGROUND OF THE INVENTION

Infectious diseases impact pig health and the stability and productivity of the global swine industry. Pig vaccination helps to prevent infections with various swine pathogens. Global regulatory agencies require that vaccine manufacturers use virus-free cell lines, including endogenous retrovirus-free cell lines in modified live viral (MLV) products. However, in many circumstances, this has not been possible. For example, swine cell lines, including PK-15 cells, harbor Porcine Endogenous Retrovirus (PERV) since it is ubiquitous in the genome of every pig. As a result, modified live viral vaccines propagated on PERV positive swine cell lines have not been allowed by some regulatory agencies in view of their guidelines, such as European Medicines Agency's (EMA) guideline EP 5.2.4 because of the presence of live contaminating virus (i.e., PERV). In particular, there are unknown implications of potential retrovirus transmission to humans when vaccines are administered to pigs or through incidental transmission to the consumer (Denner, J. One Health. 2017; 3:17-22). Inactivated viral vaccines known to contain PERV have presumably been allowed in the past by the EMA and other regulatory agencies because it has been shown the contaminating PERV is simultaneously inactivated with the product. In fact, most swine vaccines on the market today are inactivated vaccines.

Prevalent swine pathogens include, but are not limited to, porcine circovirus (PCV), porcine reproductive and respiratory syndrome virus (PRRSV), swine influenza, and Mycoplasma hyopneumoniae (M. hyopneumoniae). These porcine pathogens cause worldwide welfare and health problems in pigs of different age classes. Vaccines for use in protecting pigs against these porcine pathogens exist. However, it is known that neonatal immunity differs substantially from adults, thus different vaccines may be required for neonatal pigs. For example, overcoming maternally derived antibodies (MDA) which are present in neonatal pigs is more effective with modified live vaccines as compared to inactivated viral vaccines. Also, inactivated viral vaccines generally include an adjuvant to enhance the immune response and often require that neonatal pigs receive a prime vaccination and a booster vaccination weeks later. Therefore, a modified live PCV vaccine approach would be desired for neonatal pigs except for the potential for PERV transmission to humans.

The presence of PERVs in cells and tissues also presents a problem for pig to human transplantation of organs and tissues. The shortage of human organs for transplantation is a major barrier to the treatment of patients suffering from organ failure. Although porcine organs are considered a promising alternative, the presence of porcine endogenous retroviruses (PERVs) within the genome of every pig has raised concern (R. A. Weiss, Xenotransplantation, Vol. 25, Issue 4 2018 e12401). PERVs do not appear to do the pig any harm, and their equivalent in humans (HERV) appear to be equally benign. However, concern has been raised over the unknown implications if pig organs containing PERV are transplanted into humans. Although PERV infectivity has been documented to date only in vitro in porcine and human cell line cocultures, the possibility that xenotransplantation with porcine tissue may induce porcine retroviral expression in the recipient is of concern (Principles of Tissue Engineering (Second Edition) 2000, pages 553-558).

PERV has been divided into three subtypes within the porcine genome, A, B, and C. PERV A and B were found to infect selected human and pig cell lines, in addition to other mammalian cell types, while PERV C was restricted to replication in porcine cell lines. However, PERV C can recombine with PERV A and produce high-titer, human-tropic A/C recombinants. Thus, if xenotransplantation resulted in the expression of PERV in vivo, the risk of transmission of PERV to the recipient could raise the potential for selection and spread of new variants of PERV adapted to growth in the human host, first to immediate contacts and then to the general public (P. Gianello, in Regenerative Applications in Organ Transplantation, Chapter 69, page 963, 2014).

The potential of CRISPR-Cas9 gene editing to eliminate the risk of PERV transmission during xenotransplantation has been realized. In a breakthrough study in 2015, members of the Church lab at Harvard Medical School demonstrated the successful use of CRISPR-Cas9 gene editing to target, for the first time, all 62 copies of PERVs in a porcine cell line (Yang et al. Science 350, 1101-1104, 2015). In 2017, these authors further announced the production of healthy, PERV free pigs whose organs would be used in preclinical trials of xenotransplantation, thus eliminating the risk of PERV transmission to the organ recipient (Niu et al. Science 357 (6357), 1303-1307, 2017). Current research involves the use of CRISPR-Cas9 to improve immunocompatibility between porcine organs and humans, as has previously been demonstrated between porcine organs and NHPs (baboons) (E. Shrock and Marc Güell, in Progress in Molecular and Translational Science, (2017), Volume 152, Chapter 6, pages 106-107).

It would be desirable to propagate modified live vaccine antigens on PERV negative swine cells. A modified live vaccine grown on PERV negative swine cells could potentially make the vaccine suitable for use in neonatal pigs to overcome MDA without the need for a booster vaccination, while also eliminating the potential for PERV transmission to the humans administering the vaccine. In addition, vaccines could be produced to keep PERV free pigs intended for xenotransplantation safe from infections with swine pathogens while simultaneously keeping them PERV free.

### SUMMARY OF THE INVENTION

The present invention provides a method of preparing a vaccine. The method includes infecting gene-edited porcine endogenous retrovirus (PERV) negative swine cells with a microorganism which expresses at least one protein antigen capable of inducing protective immunity in an animal against an infectious agent; culturing the infected cells in culture medium to propagate the microorganism; and harvesting the propagated microorganism from the culture medium to obtain a fraction comprising a PERV free antigen for use in immunizing an animal against the infectious agent.

In one embodiment, the microorganism used to infect the gene-edited PERV negative cells in the above-described method need not be derived from a live organism. For example, a live virus is not required for generating replicating virus on PERV-negative cells, where it is possible to recover replicating virus following introduction of DNA or RNA encoding the viral proteins into PERV-negative cells. This particular embodiment of the method may be relevant when a wild type or recombinantly-produced whole virus is derived from pigs, cells, cell supernates, or other sources which are not PERV negative to prevent re-introduction of PERV into the PERV-negative cells.

Accordingly, the present invention further provides a method of preparing a vaccine, which includes: transfecting gene-edited PERV negative swine cells with DNA or RNA carrying sequence encoding the viral proteins required for viral packaging, assembly, and recovery of a virus; culturing the transfected cells in culture medium to propagate the virus; and recovering the propagated virus from the culture medium to obtain a fraction comprising a PERV free antigen for use in immunizing an animal against an infectious agent. In this way, live virus can be recovered from a PERV negative source (e.g., the CRISPR-edited cells described herein). Once the virus is recovered, that virus stock can be used to propagate the virus in the PERV negative cells moving forward to make more antigen, if desired.

In the passages below, it is to be understood that these embodiments may apply to each of the above-described methods of preparing a vaccine.

In one embodiment, the PERV free antigen comprised in the fraction comprising the PERV free antigen is the propagated microorganism (e.g., the propagated virus) or is derived therefrom. In another embodiment, the method further include isolating the PERV free antigen from the propagated microorganism.

In one embodiment, the PERV free antigen for use in immunizing the animal is a live antigen. In some embodiments, the method further includes lyophilizing the PERV free live antigen. In another embodiment, the method includes inactivating the PERV free live antigen with a chemical inactivant. In yet another embodiment, the method further includes removing the chemical inactivant. In a still further embodiment, the method further includes combining the inactivated PERV free antigen with an adjuvant.

In a further embodiment, the method of preparing a vaccine includes separating the PERV free antigen from cellular material. In another embodiment, the microorganism used to infect the PERV negative swine cells is contained in a cell lysate. In yet another embodiment, the method further includes employing culture medium containing the infected PERV negative swine cells as seed material for serial passages to produce a further amount of the PERV free antigen.

In further embodiments, the method of preparing a vaccine includes concentrating the PERV free antigen. In one embodiment, the PERV free antigen is concentrated by ultrafiltration. In another embodiment, the method includes washing the concentrated PERV free antigen with a balanced salt solution to reduce serum proteins.

In another embodiment, the method of preparing a vaccine further includes combining the PERV free antigen with an additional antigen. In yet another embodiment, the method includes combining the PERV free antigen with a pharmaceutically acceptable carrier.

In some embodiments, the microorganism used to infect the PERV negative swine cells is selected from the following: a porcine circovirus (PCV), porcine reproductive and respiratory syndrome virus (PRRSV), Porcine parvovirus (PPV), Influenza A Virus of Swine (IAV-S), Porcine epidemic diarrhea virus (PEDV), Swine delta coronavirus (SDCoV), Lawsonia intracellularis, Salmonella, and Bovine Viral Diarrhea Virus-Type 1 (BVDV-1) and Bovine Viral Diarrhea Virus-Type 2 (BVDV-2).

In a particular embodiment, the microorganism used to infect the PERV negative swine cells is a porcine circovirus. In one embodiment, the porcine circovirus is a wild-type virus. In another embodiment, the porcine circovirus is a chimeric whole virus. In yet another embodiment, the chimeric whole virus is a recombinant PCV1-2 virus comprising the ORF1 replicase of PCV1 and the ORF2 of a pathogenic PCV2 genotype. In one embodiment, the ORF2 is from a PCV2 genotype selected from PCV2 type 2a, PCV2 type 2b, PCV2 type 2d, and combinations thereof. In a particular embodiment, the ORF2 is from a PCV2d genotype.

In one embodiment of the method of preparing a vaccine, the PERV negative swine cells infected with the microorganism are PERV negative PK-15 cells. In a particular embodiment, the PERV negative PK-15 cells are infected with a chimeric PCV1-2 virus comprising the ORF1 replicase of PCV1 and the ORF2 of a PCV2 genotype. In one embodiment, the ORF2 in the PCV1-2 virus used to infect the PERV negative PK-15 cells is from a PCV2 genotype selected from PCV2 type 2a, PCV2 type 2b, PCV2 type 2d, and combinations thereof.

In another embodiment of the method of preparing a vaccine, the PERV negative swine cells which are infected with the microorganism are engineered to express porcine CD163.

In a particular embodiment, the PERV negative swine cells engineered to express porcine CD163 are infected with PRRS virus.

In still further embodiments of the method of preparing a vaccine, the PERV negative swine cells which are infected with the microorganism include PERV sequences disrupted at genetic locations within the PERV pol gene, wherein one of said genetic locations is within the catalytic region of the PERV pol gene and another of said genetic locations is upstream of the catalytic region of the PERV pol gene.

The present invention further provides a porcine endogenous retrovirus (PERV) negative swine cell line, wherein the cell line comprises PERV sequences disrupted at genetic locations within the PERV pol gene, wherein one of said genetic locations is within the catalytic region of the PERV pol gene and another of said genetic locations is upstream of the catalytic region of the PERV pol gene. In one embodiment, the PERV negative swine cell line is a porcine kidney (PK) cell line.

In some embodiments, the PERV negative swine cell line is produced by a method including: (a) introducing into parent swine cells (i) two guide ribonucleic acids (gRNAs) that target the PERV pol gene, wherein one of said gRNAs targets the catalytic region of the PERV pol gene and the other of said gRNAs targets a region upstream of the catalytic region of the PERV pol gene; and (ii) a nucleic acid sequence that encodes a Cas protein; (b) culturing the cells under suitable conditions in which the Cas protein is expressed and the gRNAs recruit the Cas protein to the site of targeted PERV pol gene; and (c) allowing the Cas protein to create double-stranded breaks in multiple copies of the targeted PERV pol gene, thereby inactivating expression of said copies. In one embodiment, the Cas protein is a Cas9 protein.

In some embodiments of the PERV negative swine cell line, all functional copies of the targeted PERV pol gene are inactivated. In one embodiment, the gRNA that targets the catalytic region of the PERV pol gene comprises the sequence: 5′ TACTGGAGGAGGGTCACCTG 3′ (SEQ ID NO: 6). In another embodiment, the gRNA that targets a region upstream of the catalytic region of the PERV pol gene comprises the sequence: 5′ ACCAGTACAGGACTTGAG 3′ (SEQ ID NO: 4).

The present invention further provides an allelic variant of the porcine endogenous retrovirus (PERV) pol gene wherein the allelic variant results from a disruption in the PERV pol gene at about nucleotide 437 to about nucleotide 971 of the PERV pol consensus gene represented by the sequence of SEQ ID NO: 1, and wherein said allelic variant is capable of conferring loss of PERV function upon a swine cell line. In one embodiment, the allelic variant results from a disruption in the PERV pol gene at about nucleotides 537 to about 853 of the PERV pol consensus gene represented by the sequence of SEQ ID NO: 1.

The present invention further provides a PERV free vaccine for use in protecting a pig against a swine pathogen, wherein the vaccine includes an immunogenic fraction derived from one or more microorganisms capable of infecting the PERV negative swine cell line described above. In some embodiments, the one or more microorganisms from which the immunogenic fraction is derived are selected from viruses, bacteria, and combinations thereof. In another embodiment, the one or more microorganisms from which the immunogenic fraction is derived is an obligate intracellular parasite. In one embodiment, the PERV free vaccine is for use in protecting a PERV free pig or a neonatal pig.

The present invention also provides PERV free cultures of microorganisms, wherein said PERV free cultures are propagated on the PERV free cell line described above. In one embodiment, the microorganisms are selected from viruses, bacteria, and combinations thereof.

### BRIEF DESCRIPTION OF THE DRAWINGS

FIG. 1 is a graph of the results of digital droplet PCR (ddPCR) analysis of the PK-15 parental cell line.

FIG. 2 depicts the first 1540 bases (of 3441 total bases) of the PERV polymerase gene consensus sequence SEQ ID NO: 1, as well as a schematic showing of the target sequences of candidate gRNAs 1844, 1845, 1847, and 1850, and the template sequences to which the primers used for amplification and sequencing bind.

FIG. 3A depicts guide RNA, gRNA 1844, which was selected for final editing of the PERV polymerase sequence in PK-15 cells.

FIG. 3B depicts guide RNA, gRNA 1847, which was further selected for final editing of the PERV polymerase sequence in PK-15 cells.

FIG. 4A shows TIDE analysis of the 3B7_1 (SKB7) clone at the g1847 target site.

FIG. 4B shows TIDE analysis of the 3B7_1 (SKB7) clone at the g1844 target site.

FIG. 5A shows TIDE analysis of Clone 3D8_2 at the g1847 target site.

FIG. 5B shows TIDE analysis of Clone 3D8_2 at the g1844 target site.

FIG. 6 is a schematic depiction of the plasmid construct referred to herein as pUC57+PCV1-2d, or pUC57-Kan+PCV1-2d, which included a PCV1-2d region.

FIGS. 7A, 7B, and 7C collectively depict the process of digestion of the pUC57-Kan+PCV1-2d plasmid and ligation of the cPCV1-2d sequence.

FIG. 8 is a graph of the PCV2 viremia results in Table 15.

FIG. 9 is a graph of the PCV2 fecal shedding results in Table 17.

FIG. 10 is a graph of the PCV2 nasal shedding results in Table 19.

FIG. 11 is a graph of the PCV2 viremia results in Table 26.

FIG. 12 is a graph of the PCV2 fecal shedding results in Table 28.

FIG. 13 is a graph of the PCV2 oral nasal shedding results in Table 30.

FIG. 14 is a graph summarizing the lymphoid depletion results per treatment and tissue from Table 32.

FIG. 15 is a graph summarizing the histiocytic replacement results per treatment and tissue from Table 34.

FIG. 16 is a graph summarizing the immunohistochemistry results per treatment and tissue from Table 37.

FIG. 17 is a graph depicting the PCV2 serum antibody titers by treatment and day of study from Table 44.

FIG. 18 is a graph of the PCV2 viremia results in Table 45.

FIG. 19 is a graph of the PCV2 fecal shedding results in Table 48.

FIG. 20 is a graph of the PCV2 nasal shedding results in Table 51.

FIG. 21 is a graph of SKB7 viable cell counts.

FIG. 22 is a graph of SKB7 percent viability over time.

FIG. 23 is a graph depicting cPCV1-2d live viral titers during multi-harvest growth process.

FIG. 24 is a graph of IAV-S PAH 618 growth on SKB7 cells measured by RT-qPCR.

### BRIEF DESCRIPTION OF THE SEQUENCES

SEQ ID NO: 1 is a consensus PERV Polymerase gene sequence.

SEQ ID NO: 2 is the sequence for gRNA 1848, originally disclosed by Yang et al. supra as gRNA1.

SEQ ID NO: 3 is the sequence for gRNA 1849, originally disclosed by Yang et al. supra as gRNA2.

SEQ ID NO: 4 is the sequence for gRNA 1844.

SEQ ID NO: 5 is the sequence for gRNA 1845.

SEQ ID NO: 6 is the sequence for gRNA 1847.

SEQ ID NO: 7 is the sequence for gRNA 1850.

SEQ ID NO: 8 is a PERV guide DNA sequence. Guide RNA g1844 binds to the complement of the sequence ACCAGTACAGGACTTGAGAG which is contained within SEQ ID NO: 8. SEQ ID NO: 9 is a PERV guide DNA sequence. Guide RNA 1847 binds to the sequence CAGGTGACCCTCCTCCAGTA which is contained within SEQ ID NO: 9.

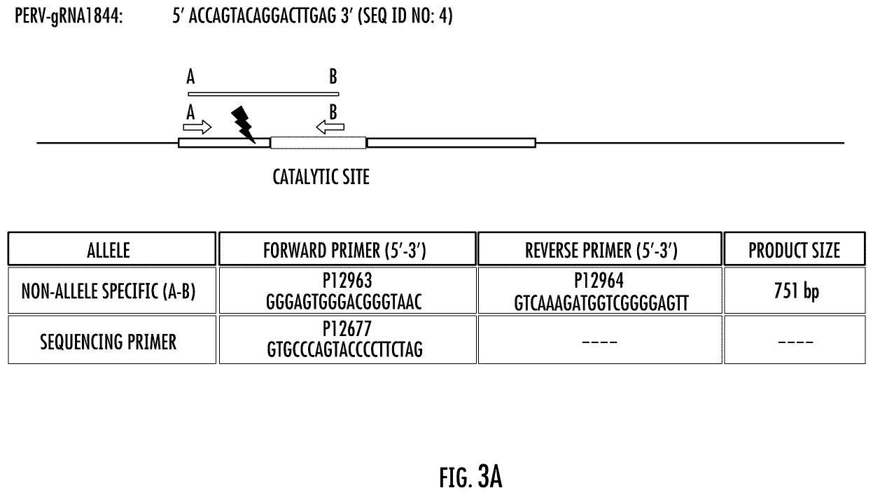

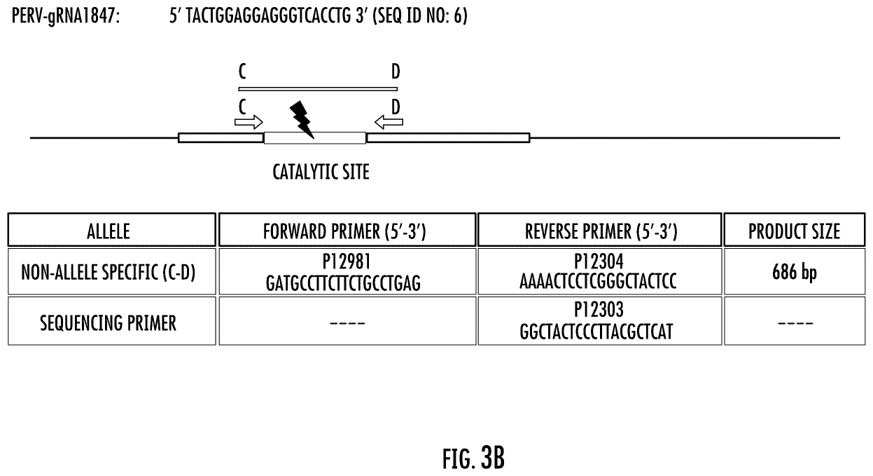

SEQ ID NO: 10 is a forward primer (5′-3′) termed P12963 used to amplify the A-B target region schematically depicted in FIG. 3A to confirm the expected product size for the allele of 751 bp.

SEQ ID NO: 11 is a reverse primer termed P12964 used to amplify the A-B target region schematically depicted in FIG. 3A to confirm the expected product size for the allele of 751 bp.

SEQ ID NO: 12 is a forward (5′-3′) sequencing primer termed P12677 in FIG. 3A, which was used to sequence the A-B PCR fragment for comparison with the wild-type sequence. SEQ ID NO: 13 is a forward primer (5′-3′) termed P12981 used to amplify the C-D target region schematically depicted in FIG. 3B to confirm the expected product size for the allele of 686 bp.

SEQ ID NO: 14 is a reverse primer termed P12304 used to amplify the C-D target region schematically depicted in FIG. 3B to confirm the expected product size for the allele of 686 bp.

SEQ ID NO: 15 is a reverse-sequencing primer termed P12303 in FIG. 3B, which was used to sequence the C-D PCR fragment for comparison with the wild-type sequence.

SEQ ID NO: 16 is the nucleotide sequence of a chimeric PCV1-2d virus described herein which includes a PCV1 replicase portion and the ORF2 of a PCV2d genotype.

SEQ ID NO: 17 is the nucleotide sequence of a chimeric PCV1-2d virus which includes a PCV1 replicase portion, the ORF2 of a PCV2d genotype and an additional repeat portion of the PCV1 replicase gene.

SEQ ID NO: 18 is the nucleotide sequence of the PCV1 ORF1 replicase portion of SEQ ID NO: 16.

SEQ ID NO: 19 is the nucleotide sequence of the PCV2d ORF2 portion of SEQ ID NO: 16.

SEQ ID NO: 20 is the amino acid sequence of the full-length ORF2 capsid protein encoded by the ORF2 gene sequence of SEQ ID NO: 18.

SEQ ID NO: 21 is the sequence of the plasmid pUC57-Kan from GenScript USA.

SEQ ID NO: 22 is the complete genome sequence of a PCV2d isolate referred to herein as “PCV2d, Isolate Z12” which was previously referred to in the art as “PCV2b-DIV-MUT”.

SEQ ID NO: 23 is the complete nucleotide sequence “DIV Clone_pUC57-Kan” received by Zoetis from GenScript.

SEQ ID NO: 24 is the nucleotide sequence of a chimeric PCV1-2a virus described herein which includes a PCV1 replicase portion and the ORF2 of a PCV2a genotype.

SQ ID NO: 25 is the nucleotide sequence of a chimeric PCV1-2b virus described herein which includes a PCV1 replicase portion and the ORF2 of a PCV2b genotype.

SEQ ID NO: 26 is a forward primer used to PCR amplify Influenza A Virus, Swine (IAV-S) in Example 7.

SEQ ID NO: 27 is a reverse primer used to PCR amplify Influenza A Virus, Swine (IAV-S) in Example 7.

SEQ ID NO: 28 is a probe used to confirm the successful PCR amplification of Influenza A Virus, Swine (IAV-S) in Example 7.

SEQ ID NO: 29 is a forward primer used to PCR amplify BVDV in Example 8.

SEQ ID NO: 30 is a reverse primer used to PCR amplify BVDV in Example 8.

SEQ ID NO: 31 is a forward primer used to PCR amplify the Porcine Epidemic Diarrhea Virus (PEDV) in Example 9.

SEQ ID NO: 32 is a reverse primer used to PCR amplify the PEDV in Example 9.

SEQ ID NO: 33 is a probe used to confirm the successful PCR amplification of PEDV in Example 9.

### DETAILED DESCRIPTION OF THE INVENTION

As used in the specification and claims, the singular form “a”, “an” and “the” include plural references unless the context clearly dictates otherwise. For example, the term “a protein antigen” includes a plurality of protein antigens, including mixtures thereof. As used herein, the term “comprising” is intended to mean that the compositions and methods include the recited elements, but do not exclude other elements.

The term clustered regularly interspaced palindromic repeats (CRISPR)/Cas 9 is a gene editing technology that makes it possible to correct errors in the genome and turn on or off genes in cells and organisms. As particularly used herein, CRISPR/Cas 9 was used to inactivate the catalytic activity of the retroviral polymerase protein encoded by the polymerase (pol) gene of a porcine endogenous retrovirus, thus making it impossible for PERV to replicate within a host.

As used herein, the term “porcine endogenous retrovirus (PERV) negative swine cells” “PERV negative swine cell line” and the like is intended to mean that all functional copies of the PERV pol gene within the swine cells or swine cell line are inactivated. In one embodiment, all the targeted PERV copies carry out-of-frame or in-frame modifications in the catalytic region that would either create a frame shift within the protein coding sequence and likely generate an early stop codon or disable the catalytic activity of the retroviral polymerase protein within the swine cells or swine cell line following deletion of essential codons.

As used herein, the term “PCV2d” refers to a PCV2 genotype that is believed to have diverged from the PCV2b genotype. Before the nomenclature was established and accepted, a PCV2d strain was alternatively referred to in the art as “PCV2b divergent strain”, “PCV2b divergent”, “PCV2 mutant”, “novel mutant PCV2”, “mutant PCV2”, and the like. PCV2d encodes an ORF2 capsid polypeptide that includes Asparagine (N) at position 68, Leucine (L) at position 89, Threonine (T) at position 90, and Asparagine (N) at position 134, according to the numbering of SEQ ID NO: 20. SEQ ID NO: 20 is an ORF2 polypeptide encoded by a representative PCV2d strain referred to herein as “PCV2d, Isolate Z12” which was previously referred to in the art as “PCV2b-DIV-MUT” whose genomic sequence corresponds to SEQ ID NO: 22. The encoded PCV2d ORF2 polypeptide can further include at least one residue selected from: a Lysine (K) at residue 59, a Lysine (K) at residue 234, a Threonine (T) at residue 190, an Isoleucine (I) at residue 53, an Arginine (R) or Glycine (G) at residue 169, and an Isoleucine (I) at residue 215 according to the numbering of SEQ ID NO: 20.

As used herein, the term “chimeric PCV1-2 virus” is intended to refer to a virus comprising and/or expressing the ORF1 replicase of PCV1 and the ORF2 of PCV2, wherein the ORF2 of PCV2 can be derived from a PCV2 genotype, including but not limited to PCV2 type 2a, PCV2 type 2b, PCV2 type 2d, and combinations thereof. In such embodiments, the chimeric PCV1-2 virus may be referred to herein as chimeric PCV1-2a, chimeric PCV1-2b, and chimeric PCV1-2d, respectively, and the like, depending on the PCV2 genotype from which the ORF2 portion is derived. The chimeric PCV1-2 virus contains a nucleotide sequence encoding the capsid protein of a PCV2 genotype, such as PCV2 type 2d (PCV2d) in the genomic backbone of PCV1 in place of the sequence encoding the ORF2 capsid protein of PCV1. The PCV2 ORF2 polypeptide is expressed by the virus, such that the ORF2 polypeptide is a component of the virus itself (e.g., protein coat of the virus). The terms “chimeric PCV1-2d”, “cPCV1-2d”, “PCV1-2d chimeric whole virus”, “PCV1-2d, “chimeric PCV1-2d vaccine” and the like may be used interchangeably herein to refer to a virus comprising and/or expressing the ORF1 of PCV1 and the ORF2 of the PCV2d genotype or to a vaccine comprising such a virus.

The term “antigen”, “protein antigen” and the like refers to a compound, composition, or immunogenic substance that can stimulate the production of antibodies or a T-cell response, or both, in an animal, including compositions that are injected or absorbed into an animal. The immune response may be generated to the whole molecule, or to a portion of the molecule (e.g., an epitope or hapten). The term “antigen” can include a whole virus, a polypeptide, such as a protein, or a fragment thereof.

The term “PERV free antigen” is intended to mean an antigen or antigenic fraction which has been made using swine cells in which the catalytic activity of the retroviral polymerase protein of PERV has been inactivated.

The term “microorganism” as used herein is an organism that can be seen only through a microscope, which may be single celled or multicellular. Microorganisms include, but are not limited to bacteria, viruses, and protozoa, as well as multicellular animal parasites (helminths). The term “microorganism” as used herein for use in infecting the gene-edited PERV negative cells is intended to include a whole microorganism (e.g., wild-type or recombinantly-produced), as well as a microbial vector carrying a nucleic acid sequence encoding an immunogenic protein capable of eliciting protective immune response to a viral, bacterial, or protozoal strain(s) or types disclosed herein, A vaccine comprising such a microbial vector may be alternatively referred to herein as a vectored vaccine. Microbial vectors include a viral vector such as, for example, a canarypox vector, an adenovirus vector or a vaccinia vector, or a bacterial vector such as Salmonella, E. coli, or other bacteria), wherein the viral or bacterial vector carries a nucleic acid sequence encoding the immunogenic protein. In one embodiment, a vectored vaccine can be used alone and be the only part of the vaccine produced in PERV negative cells according to the methods of the present invention (e.g., for a viral vector or obligate/facultative intracellular bacterial vector). In another embodiment, a vectored vaccine could be made in a defined media lacking any cells at all (e.g., for a bacterial vector), optionally inactivated, and then used in combination with an antigen produced in the PERV negative cells according to the methods of the present invention.

As used herein, the term “intracellular parasite” refers to both obligate intracellular parasites and facultative intracellular parasites. Obligate intracellular parasites are microorganisms that cannot reproduce outside their host cell, which means that the microorganism is entirely reliant on intracellular resources. Facultative intracellular parasites are microorganisms that can utilize intracellular resources to reproduce inside a cell, but that can also reproduce outside their host cell (e.g., Salmonella spp., Listeria spp.). Obligate intracellular parasites include all viruses, as well as certain bacteria, for example Chlamydia and Rickettsia, or Protozoa (e.g., Plasmodium spp., Toxoplasma gondii, Cryptosporidium parvum, and Leishmania spp). However, it is to be understood that the present invention is not limited to the use of intracellular parasites. For example, as described above, additional antigens which may be added to a PERV free vaccine composition prepared as described herein can include, but are not limited to, live or inactivated microbial antigens which are capable of growing and reproducing without a host cell. As another non-limiting example described above, the antigen component may comprise a microbial vector, such as a viral vector or a bacterial vector carrying a nucleic acid sequence encoding an immunogenic protein capable of eliciting protective immune response to a viral, bacterial, or protozoal strain(s) or types disclosed herein. In one embodiment, no live or inactivated antigens included in the final vaccine are grown in PERV positive cells. In another embodiment, an antigen grown in PERV positive cells may be combined with an antigen produced in PERV negative cells as described herein if it were inactivated since the contaminating PERV would be simultaneously inactivated.

As used herein, the term “vaccine composition”, “vaccine” and the like refers to a composition which includes at least one antigen or immunogen in a pharmaceutically acceptable vehicle useful for inducing an immune response in a host. Vaccine compositions can be administered in dosages, and by techniques well known to those skilled in the medical or veterinary arts, taking into consideration factors such as the age, sex, weight, species and condition of the recipient animal, and the route of administration. The route of administration can be percutaneous, via mucosal administration (e.g., oral, nasal, anal, vaginal) or via a parenteral route (intradermal, transdermal, intramuscular, subcutaneous, intravenous, or intraperitoneal). Vaccine compositions can be administered alone, or can be co-administered or sequentially administered with other treatments or therapies. Forms of administration may include suspensions, syrups or elixirs, and preparations for parenteral, subcutaneous, intradermal, intramuscular or intravenous administration (e.g., injectable administration) such as sterile suspensions or emulsions. Vaccine compositions may be administered as a spray, or mixed in food and/or water, or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired. Standard pharmaceutical texts, such as “Remington's The Science and Practice of Pharmacy 23rd Edition” (Oct. 30, 2020), may be consulted to prepare suitable preparations, without undue experimentation.

“Needle-free administration”, “needle-free vaccine delivery” and the like is intended to mean that a vaccine is delivered to the animal through a jet injector which is a needle-free device that delivers liquid vaccine through a nozzle orifice and penetrates the skin of the animal with a high-speed narrow stream. As disclosed herein, a cPCV1-2d vaccine propagated on PERV negative swine cells was suitably administered intramuscularly to piglets via needle as well as needle-free vaccine delivery. The piglets elicited a protective immune response in response to both the needle and needle-free delivery.

The term “immune response” as used herein refers to a response elicited in an animal. An immune response may refer to cell-mediated immunity (CMI), humoral immunity, or may involve both. The present invention also contemplates a response limited to a part of the immune system. Usually, an “immunological response” includes, but is not limited to, one or more of the following effects: the production or activation of antibodies, B cells, helper T cells, suppressor T cells, and/or cytotoxic T cells and/or γδ T cells, directed specifically to an antigen or antigens included in the composition or vaccine of interest.

The term “protective immunity” and the like refers to either a therapeutic or protective immunological response elicited in an animal host, such that resistance to new infection will be enhanced, and/or the clinical severity of the disease reduced. Such protection will be demonstrated by either a reduction or lack of symptoms normally displayed by an infected host, a quicker recovery time, and/or a lowered viral titer in the infected host.

As used herein, the term “immunogenicity” means capable of producing an immune response in a host animal against an antigen or antigens. This immune response forms the basis of the protective immunity elicited by a vaccine against a specific infectious organism.

An “adjuvant” as used herein means a composition comprised of one or more substances that enhances the immune response to an antigen(s). The mechanism of how an adjuvant operates is not entirely known. Some adjuvants are believed to enhance the immune response by slowly releasing the antigen, while other adjuvants are strongly immunogenic in their own right and are believed to function synergistically through selective interaction and activation of toll-like receptors (TLR) which induce cellular signaling cascades and generation of immune response.

As used herein, the term “multivalent” means a vaccine containing more than one antigen, whether from the same microbiological species (e.g., different isolates of M. hyopneumoniae or PCV), from different species (e.g., isolates from both Pasteurella hemolytica and Pasteurella multocida), or a vaccine containing a combination of antigens from different genera (for example, a vaccine comprising antigens from Pasteurella multocida, Salmonella, Escherichia coli, Haemophilus somnus and Clostridium).

The term “pig” or “piglet” as used herein means an animal of porcine origin, while “sow” refers to a female pig of reproductive age and capability. A “gilt” is a female pig who has never been pregnant. A “neonatal pig” is a newborn piglet, typically between 1 to 3 days of age. Neonatal piglets are considered to have an immature but rapidly developing adaptive immune system. Neonatal piglets are capable of innate immune responses, and inherit short-term passive protection imparted by cells and antibodies transferred during gestation and through milk/colostrum ingestion during early growth. Accordingly, the ability to obtain a consistent and protective immune response in neonatal piglets is often more difficult than in older pigs.

As used herein, the term “virulent” means an isolate that retains its ability to be infectious in an animal host and is capable of causing disease in the host animal.

“Inactivated vaccine” means a vaccine composition containing an infectious organism or pathogen that is no longer capable of replication or growth. The pathogen may be bacterial or viral in origin and/or may be an obligate intracellular parasite, which as described herein refers to microorganisms, such as all viruses and certain bacteria that cannot reproduce outside their host cell. Additionally, the pathogen may be a facultative intracellular parasite or a viral vector or bacterial vector which is treated in such a way as to render it unable to replicate. Inactivation may be accomplished by a variety of methods, including freeze-thawing, chemical treatment (for example, treatment with β-propiolactone (BPL) or formalin), sonication, radiation, heat, or any other conventional means sufficient to prevent replication or growth of the organism, while maintaining its immunogenicity.

A “balanced salt solution” is a solution made to a physiological PH and isotonic salt concentration.

The term “variant” as used herein refers to a polypeptide or a nucleic acid sequence encoding a polypeptide, that has one or more conservative amino acid variations or other minor modifications such that the corresponding polypeptide has substantially equivalent function when compared to the wild-type polypeptide. The term “variant” can also refer to a microorganism comprising a polypeptide or nucleic acid sequence having said variations or modifications as well.

“An allelic variant” as used herein refers to any of two or more variants of a gene that have the same relative position on homologous chromosomes and are responsible for alternative characteristics, such as a full-length or truncated version of a protein.

“Conservative variation” denotes the replacement of an amino acid residue by another biologically similar residue, or the replacement of a nucleotide in a nucleic acid sequence such that the encoded amino acid residue does not change or is another biologically similar residue. Examples of conservative variations include the substitution of one hydrophobic residue, such as isoleucine, valine, leucine or methionine, for another hydrophobic residue, or the substitution of one polar residue with another, such as the substitution of arginine for lysine, glutamic acid for aspartic acid, or glutamine for asparagine, and the like. The term “conservative variation” also includes a substituted amino acid in place of a parent amino acid, provided that antibodies raised to the substituted polypeptide also immunoreact with the parent (unsubstituted) polypeptide.

As used herein, the terms “pharmaceutically acceptable carrier”, “pharmaceutically acceptable vehicle”, “veterinary acceptable carrier” and the like refer to a fluid vehicle for containing vaccine antigens that can be injected into a host, such as an animal host, without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, coloring additives, and the like.

“North American PRRS virus” means any PRRS virus having genetic characteristics associated with a North American PRRS virus isolate, such as, but not limited to, the PRRS virus that was first isolated in the United States around the early 1990's (see, e.g., Collins, J. E., et al., 1992, J. Vet. Diagn. Invest. 4:117-126); North American PRRS virus isolate MN-1b (Kwang, J. et al., 1994, J. Vet. Diagn. Invest. 6:293-296); the Quebec LAF-exp91 strain of PRRS virus (Mardassi, H. et al., 1995, Arch. Virol. 140:1405-1418); and North American PRRS virus isolate VR 2385 (Meng, X.-J et al., 1994, J. Gen. Virol. 75:1795-1801). Additional examples of North American PRRS virus strains are known in the art. Genetic characteristics refer to genomic nucleotide sequence similarity and amino acid sequence similarity shared by North American PRRS virus strains. “Chinese PRRS virus” strains generally evidence about 80-93% nucleotide sequence similarity with North American strains.

“European PRRS virus” refers to any strain of PRRS virus having the genetic characteristics associated with the PRRS virus that was first isolated in Europe around 1991 (see, e.g., Wensvoort, G., et al., 1991, Vet. Q. 13:121-130). “European PRRS virus” is also sometimes referred to in the art as “Lelystad virus”. Further examples of European PRRS virus strains are known in the art.

“CD163” is a macrophage differentiation antigen belonging to the scavenger receptor cysteine-rich (SRCR) family of membrane proteins. CD163 is a cellular receptor capable of mediating infection of otherwise PRRSV non-permissive cell lines.

As used herein, a genetically modified virus is “attenuated” if it is less virulent than its unmodified parental strain. A strain is “less virulent” if it shows a statistically significant decrease in one or more parameters determining disease severity. Such parameters may include level of viremia, fever, severity of respiratory distress, severity of reproductive symptoms, or number or severity of pathological lesions, etc.

An “infectious clone” is an isolated or cloned genome of the disease agent (e.g. viruses) that can be specifically and purposefully modified in the laboratory, and then used to re-create the live genetically-modified organism. A live genetically-modified virus produced from the infectious clone can be employed in a live viral vaccine. Alternatively, inactivated virus vaccines can be prepared by treating the live virus derived from the infectious clone with inactivating agents such as formalin, beta-propriolactone, binary ethylenemine or hydrophobic solvents, acids, etc., by irradiation with ultraviolet light or X-rays, by heating, etc.

The present invention provides a method of preparing a vaccine composition. The method includes infecting gene-edited porcine endogenous retrovirus (PERV) negative swine cells with a microorganism which expresses at least one protein antigen capable of inducing protective immunity in an animal against an infectious agent; culturing the infected cells in culture medium to propagate the microorganism; and harvesting the propagated microorganism from the culture medium to obtain a fraction comprising a PERV free antigen for use in immunizing an animal against the infectious agent. In one embodiment, the PERV free antigen comprised in the fraction is the propagated microorganism or is derived therefrom. In another embodiment, the method of preparing a vaccine composition further includes isolating the PERV free antigen from the propagated microorganism.

In some embodiments, the PERV free antigen used to immunize an animal is a modified live antigen. In one embodiment, the PERV free modified live antigen is in a lyophilized form and can be rehydrated with a liquid prior to administration to the animal subject. The liquid used to rehydrate the PERV free antigen can optionally include one or more additional antigens such that the rehydration results in a suitable multivalent vaccine.

In another embodiment, the PERV free live antigen may be inactivated, such as with a chemical inactivant. In one embodiment, the method further includes removing the chemical inactivant.

It is envisioned that a PERV free modified live vaccine composition can be made in accordance with the method of the present invention and thus satisfy global regulatory agencies which require that vaccine manufacturers use virus-free cell lines in their manufacturing process, including endogenous retrovirus-free cell lines in modified live products. Alternatively, a PERV free live antigen may be inactivated as part of the method, if desired, and optionally combined with an adjuvant. For example, as will be explained in further detail below, in some embodiments the PERV free antigen may be combined with an additional antigen to form a multivalent vaccine. In some embodiments, it may be desirable to combine a PERV free inactivated antigen made as described herein with another inactivated antigen rather than having one antigen be live and the other inactivated.

It is further envisioned that a modified live PERV free antigen prepared in accordance with the method of the present invention can be combined with one or more inactivated vaccines provided the adjuvant in the inactivated vaccine is not detrimental to the live PERV free antigen, and provided the combined antigenic fractions are otherwise compatible with each other. This is irrespective of whether the one or more inactivated vaccines which are to be combined with the live PERV free antigen are PERV free or not.

A significant advantage of employing a modified live PERV free antigen is that it can be given to younger piglets (1-3 days of age) since live antigen compositions typically do not include adjuvants. In addition, an immune response elicited in an animal immunized with a live antigen typically provides the same immunity, including both cellular mediated immunity (CMI) and humoral immunity, produced by natural exposure. Because animals immunized with modified live antigens typically have sterilizing immunity that prevents clinical disease and infection, a booster (i.e., second dose) may not be needed. Also, the immune response elicited in younger piglets vaccinated with modified live antigens should ideally overcome maternally derived antibodies which are typically present in younger piglets.

In a further embodiment, the method includes separating the PERV free antigen from cellular material, although this may not be necessary. In another embodiment, the microorganism used to infect the PERV negative swine cells is contained in a cell lysate. In yet another embodiment, the method further includes employing culture medium containing the infected PERV negative swine cells as seed material for serial passages to produce a further amount of the PERV free antigen.

In further embodiments, the method of preparing a vaccine includes concentrating the PERV free antigen. In one embodiment, the PERV free antigen is concentrated by ultrafiltration. In another embodiment, the method includes washing the concentrated PERV free antigen with a balanced salt solution to reduce serum proteins.

As described above, in one embodiment, the method of preparing a vaccine further includes combining the PERV free antigen with an additional antigen. In yet another embodiment, the method includes combining the PERV free modified live antigen with a pharmaceutically acceptable carrier.

In some instances, it may be desirable to combine a modified live PERV free antigen with an additional modified live PERV free antigen, wherein both have been prepared in accordance with the methods described herein. However, the present invention is not limited in this respect. For example, a liquid vaccine comprising an inactivated additional antigen can be used to rehydrate a lyophilized PERV free antigen prepared in accordance with the methods described herein.

In some embodiments, the microorganism used to infect the PERV negative swine cells is selected from the following: a porcine circovirus (PCV), porcine reproductive and respiratory syndrome virus (PRRSV), Porcine parvovirus (PPV), Influenza A Virus of Swine (IAV-S), Porcine epidemic diarrhea virus (PEDV), Swine delta coronavirus (SDCoV), Lawsonia intracellularis, Salmonella, and Bovine Viral Diarrhea Virus-Type 1 (BVDV-1) and Bovine Viral Diarrhea Virus-Type 2 (BVDV-2).

In a particular embodiment, the microorganism used to infect the PERV negative swine cells is a porcine circovirus. In one embodiment, the porcine circovirus is a wild-type virus. In another embodiment, the porcine circovirus is a chimeric whole virus. In yet another embodiment, the chimeric whole virus is a recombinant PCV1-2 virus comprising the ORF1 replicase of PCV1 and the ORF2 of a pathogenic PCV2 genotype. In one embodiment, the ORF2 is from a PCV2 genotype selected from PCV2 type 2a, PCV2 type 2b, PCV2 type 2d, and combinations thereof. In a particular embodiment, the ORF2 is from a PCV2d genotype.

In a further embodiment, a whole virus is not required for initial infection of PERV negative cells, where it is possible to recover replicating virus following introduction of DNA or RNA encoding the viral proteins into PERV negative cells. This particular embodiment of the method is important when a wild type or chimeric whole virus is derived from pigs, cells, cell supernates, or other sources which are not PERV negative to prevent re-introduction of PERV into the PERV negative cells.

In one embodiment, the PERV negative swine cells infected with the microorganism are PERV negative PK-15 cells. In a particular embodiment, the PERV negative PK-15 cells are infected with a recombinant PCV1-2 virus comprising the ORF1 replicase of PCV1 and the ORF2 of a PCV2 genotype. In one embodiment, the ORF2 in the PCV1-2 virus used to infect the PERV negative PK-15 cells is from a PCV2 genotype selected from PCV2 type 2a, PCV2 type 2b, PCV2 type 2d, and combinations thereof.

In another embodiment, the PERV negative swine cells which are infected with the microorganism are engineered to express porcine CD163. In a particular embodiment, the PERV negative swine cells engineered to express porcine CD163 are infected with PRRS virus. CD163 has been identified as the essential receptor for PRRS virus. CD163 is capable of mediating infection of otherwise PRRSV non-permissive cell lines. In one embodiment, the PRRS virus is a North American PRRS virus which has been engineered to express porcine CD163. In another embodiment, the PRRS virus is a European PRRS virus.

In still further embodiments, the PERV negative swine cells which are infected with the microorganism include PERV sequences disrupted at genetic locations within the PERV pol gene, wherein one of said genetic locations is within the catalytic region of the PERV pol gene and another of said genetic locations is upstream of the catalytic region of the PERV pol gene.

The present invention further provides a porcine endogenous retrovirus (PERV) negative swine cell line, wherein the cell line comprises PERV sequences disrupted at genetic locations within the PERV pol gene, wherein one of said genetic locations is within the catalytic region of the PERV pol gene and another of said genetic locations is upstream of the catalytic region of the PERV pol gene. In one embodiment, the PERV negative swine cell line is a porcine kidney (PK) cell line.

In some embodiments, the PERV negative swine cell line is produced by a method including: (a) introducing into parent swine cells (i) two guide ribonucleic acids (gRNAs) that target the PERV pol gene, wherein one of said gRNAs targets the catalytic region of the PERV pol gene and the other of said gRNAs targets a region upstream of the catalytic region of the PERV pol gene; and (ii) a nucleic acid sequence that encodes a Cas protein; (b) culturing the cells under suitable conditions in which the Cas protein is expressed and the gRNAs recruit the Cas protein to the site of targeted PERV pol gene; and (c) allowing the Cas protein to create double-stranded breaks in multiple copies of the targeted PERV pol gene, thereby inactivating expression of said copies. In one embodiment, the Cas protein in a Cas9 protein.

In some embodiments of the PERV negative swine cell line, all functional copies of the targeted PERV pol gene are inactivated. In one embodiment, the gRNA that targets the catalytic region of the PERV pol gene comprises the sequence: 5′ TACTGGAGGAGGGTCACCTG 3′ (SEQ ID NO: 6). In another embodiment, the gRNA that targets a region upstream of the catalytic region of the PERV pol gene comprises the sequence: 5′ ACCAGTACAGGACTTGAG 3′ (SEQ ID NO: 4).

The present invention further provides a PERV free vaccine for use in protecting a pig against a swine pathogen, wherein the vaccine includes an immunogenic fraction derived from one or more microorganisms capable of infecting the PERV negative swine cell line described above. In some embodiments, the one or more microorganisms from which the immunogenic fraction is derived are selected from viruses, bacteria, and combinations thereof. In another embodiment, the one or more microorganisms from which the immunogenic fraction is derived is an obligate intracellular parasite. In one embodiment, the PERV free vaccine is for use in protecting a PERV free pig or a neonatal pig.

The present invention also provides PERV free cultures of microorganisms, wherein said PERV free cultures are propagated on the PERV negative cell line described above. In one embodiment, the microorganisms are selected from viruses, bacteria, and combinations thereof.

The present invention further provides an allelic variant of the porcine endogenous retrovirus (PERV) pol gene wherein the allelic variant results from a disruption in the PERV pol gene at about nucleotide 437 to about nucleotide 971 of the PERV pol consensus gene represented by the sequence of SEQ ID NO: 1, and wherein said allelic variant is capable of conferring loss of PERV function upon a swine cell line. In one embodiment, the allelic variant results from a disruption in the PERV pol gene at about nucleotides 537 to about 853 of the PERV pol consensus gene represented by the sequence of SEQ ID NO: 1.

In one embodiment, a chimeric PCV1-2d whole virus-based vaccine composition may be prepared in accordance with the vaccine manufacturing method according to the present invention. In such an instance, a live version of the PCV1-2d chimeric whole virus is employed as the microorganism used to infect gene-edited PERV negative swine cells. The PCV1 replicase portion of the chimeric virus is used as the means to express the ORF2 capsid protein, which as known in the art is the major immunodominant protein of PCV2.

The examples support the use of a chimeric PCV1-2d virus (a microorganism) to infect gene-edited PERV negative cells; and the culturing of those infected cells in a suitable culture medium to propagate the virus. The propagated chimeric PCV1-2d virus was thereafter harvested from the culture medium to obtain a fraction including a PERV free antigen which in this instance was the propagated chimeric PCV1-2d virus for use in immunizing a piglet, such as including, but not limited to, a neonatal pig, against PCV2. The PERV free modified live PCV1-2d vaccine was made using gene-edited PERV negative swine cells that had been gene edited using CRISPR/Cas technology to inactivate the PERV. The PCV1-2d chimeric vaccine made in accordance with the method of the present invention was shown to elicit a protective immune response in neonatal pigs that received the vaccine and without the need for a booster vaccination, and this protection was observed even in the presence of maternally derived antibodies (MDAs).

The examples further support that microorganisms in addition to the PCV1-2d chimeric virus can be grown on the PERV negative swine cells. These other microorganisms included a PCV1-2a chimeric virus, a PCV1-2b chimeric virus, a wild type PCV2d field virus, an Influenza A Virus of Swine (IAV-S), BVDV-1a, BVDV-1b and BVDV-2. Regarding the BVDV examples, it is noted that they support that BVDV-1a, BVDV-1b and BVDV-2 were capable of growing on the CRISPR-edited PERV negative swine cells (e.g., SKB7 cells). Therefore, the method of the present invention is not limited to preparation of a swine vaccine composition.

In some embodiments, a modified live PERV free antigen for use in combination with the PERV free cPCV1-2d vaccine antigen described herein can be selected from one or more of the following: a porcine circovirus (PCV), porcine reproductive and respiratory syndrome virus (PRRSV), Porcine parvovirus (PPV), Influenza A Virus of Swine (IAV-S), Porcine epidemic diarrhea virus (PEDV), Swine delta coronavirus (SDCoV), Lawsonia intracellularis and Salmonella. As described above, the examples support that not only cPCV1-2d, but also cPCV1-2a, cPCV1-2b, a PCV2d field virus, and Influenza A Virus of Swine (IAV-S) are capable of being propagated on the PERV negative swine cells described herein, and it is reasonably expected that the other swine pathogens can be likewise grown on PERV negative swine cells. For example, PEDV and SDCoV have been shown to grow on LLC-PK1 cells (Xiao et al., (2021). Arch Virol. 166 (3): 935-941). Also, LLC-PK cells expressing CD163 have been shown to be permissible to PRRS infection and supported PRRS replication (Patton et al., (2009). Virus Research 140:161-171). Furthermore, Dulac-pCD163 cells have been shown to support PRRS replication (Research Report submitted by investigator Dongwan Yoo (University of Illinois) on May 23, 2008, to the National Pork Board, available at www.porkcheckoff.org/research/development-of-stable-cell-lines-permissive-for-prrsv-replication-and-production/. Also, PPV was shown to induce activation of NF-κB signaling pathways in PK-15 cells mediated by toll-like receptors (Cao et al., (2017). Mol Immunol., 85:248-255). In addition, a study has shown interaction, survival, and propagation of L. intracellularis in swine macrophages (Pereira et al., (Jul. 31, 2020). PLoS One. 15 (7): e0236887 pp. 1-10).

The PCV1-2d vaccine composition prepared in accordance with the method of the present invention is for protecting pigs against PCV2d, as well as for cross-protecting against other PCV2 genotypes, including PCV2b. The PCV2d ORF2 portion was derived from the strain referred to herein as PCV2d, Isolate Z12. However, the source from which the PCV2d ORF2 can be derived is not limited to this PCV2d strain. For example, the PCV2d ORF2 gene can be derived from other representative PCV2d strains which include, but are not limited to, the following:
PCV2 strain: 798-1, with GenBank Accession number AB462384,
PCV2 strain: FF, with GenBank Accession number DQ23151,
PCV2 strain: VC 2002-k2, with GenBank Accession number EF990645,
PCV2 isolate: GY09, with GenBank Accession number GQ845025,
PCV2 isolate: XS09, with GenBank Accession number GQ845028,
PCV2 isolate: SDId01, with GenBank Accession number HM535640,
PCV2 isolate: SDId02, with GenBank Accession number HM755880,
PCV2 isolate: HM01, with GenBank Accession number HM755881,
PCV2 strain: NIVS-1, with GenBank Accession number HQ378157,
PCV2 isolate: C/2010-2*, with GenBank Accession number JF683394,
PCV2 isolate: G/2009-2, with GenBank Accession number JF683408,
PCV2 isolate: I/2010, with GenBank Accession number JF927984,
PCV2 isolate: J/2010, with GenBank Accession number JF927985,
PCV2 isolate: K/2010, with GenBank Accession number JF927986,
PCV2 isolate: M/2010, with GenBank Accession number JF927988,
PCV2 isolate: WB/ROM89, with GenBank Accession number JN006445,
PCV2 isolate: EU-RO-F4-3, with GenBank Accession number JN382188,
PCV2 isolate: HNing09, with GenBank Accession number JN411096,
PCV2 isolate: YWu09, with GenBank Accession number JN411099,
PCV2 isolate: CH-IVT4, with GenBank Accession number JX984586,
PCV2 isolate: CH-IVT6, with GenBank Accession number JX984588,
PCV2 isolate: CH-IVT7, with GenBank Accession number JX984589,
PCV2 isolate, with GenBank Accession number JX984590,
PCV2 isolate: CH-IVT9, with GenBank Accession number JX984591,
PCV2 isolate: CH-IVT10, with GenBank Accession number JX984592,
PCV2 isolate: CH-IVT11, with GenBank Accession number JX984593,
PCV2 isolate: GDYX, with GenBank Accession number JX519293, and
PCV2 isolate: GDYX, with GenBank Accession number JX519293.

Each of the above PCV2d isolates comprises a genomic sequence which encodes and/or expresses an ORF2 polypeptide sequence bearing the signature ORF2 amino acid residues of the PCV2d genotype, wherein the ORF2 polypeptide comprises Leucine (L) at position 89, Threonine (T) at position 90, and Asparagine (N) at position 134, according to the numbering of SEQ ID NO: 20. As described above, this PCV2d ORF2 polypeptide can further include at least one residue selected from the following: a Lysine (K) at residue 59, a Lysine (K) at residue 234, a Threonine (T) at residue 190, an Isoleucine (I) at residue 53, an Asparagine (N) at residue 68, an Arginine (R) or Glycine (G) at residue 169, and an Isoleucine (I) at residue 215, according to the numbering of SEQ ID NO: 20.

In one embodiment, a PCV2d ORF2 polypeptide encoded and/or expressed by a PCV1-2d chimeric virus prepared in accordance with the method of the present invention includes Leucine (L) at position 89, Threonine (T) at position 90, Asparagine (N) at position 134, Lysine (K) at residue 59 and a Lysine (K) at residue 234, according to the numbering of SEQ ID NO: 20.

In a further embodiment, a PCV2d ORF2 polypeptide encoded and/or expressed by a PCV1-2d chimeric virus prepared in accordance with the method of the present invention includes Leucine (L) at position 89, Threonine (T) at position 90, Asparagine (N) at position 134, a Lysine (K) at residue 59 and a Lysine (K) at residue 234, a Threonine (T) at residue 190, an Isoleucine (I) at residue 53, an Asparagine (N) at residue 68, an Arginine (R) or Glycine (G) at residue 169, and an Isoleucine (I) at residue 215, according to the numbering of SEQ ID NO: 20.

As described above, in one embodiment, a vaccine composition made by the method of the present invention includes at least one additional antigen. In one embodiment, the at least one additional antigen is protective against a disease in pigs caused by a microorganism. In one embodiment, the at least one additional antigen can be a modified live PERV free antigen or an inactivated PERV free antigen prepared in accordance with the method described herein, but the invention is not limited in this way.

In some embodiments, at least one additional antigen component is protective against a disease in pigs caused by bacteria, viruses, or protozoans that are known to infect pigs. Examples of such microorganisms include, but are not limited to, the following: other porcine circoviruses (PCV) (e.g., 1, 3, 4), Mycoplasma hyopneumoniae, porcine reproductive and respiratory syndrome virus (PRRSV), porcine parvovirus (PPV), Haemophilus (Glaesserella) parasuis, Pasteurella multocida, Streptococcus suis, Staphylococcus hyicus, Actinobacillus pleuropneumoniae, Bordetella bronchiseptica, Salmonella choleraesuis, Salmonella enteritidis, Erysipelothrix rhusiopathiae, Mycoplasma hyorhinis, Mycoplasma hyosynoviae, Leptospira spp., Lawsonia intracellularis, Influenza A Virus of Swine (IAV-S), Escherichia coli, Brachyspira hyodysenteriae, porcine respiratory coronavirus, Porcine Epidemic Diarrhea (PED) virus, Swine delta coronavirus (SDCoV), porcine rotavirus (e.g., groups A, B, and C), Porcine Cytomegalovirus, Porcine enteroviruses, Classical Swine fever (CSF), or combinations thereof.

In other embodiments, the PERV free antigen prepared by the method of the invention and the additional antigen are both PCV2 antigens protective against porcine circovirus-associated disease (PCVAD). By way of example only, a PERV free chimeric PCV1-2d vaccine prepared by the method disclosed herein can be combined with another PCV1-2 chimeric vaccine, such as a PCV1-2b or PCV1-2a vaccine. Preferably, each of these PCV1-2 chimeric vaccines will have been made using the SKB7 cells described herein so that each is PERV free. However, if the additional antigen has been inactivated, then any contaminating PERV is simultaneously inactivated. That said, a clear advantage of employing PERV free modified live vaccines such as PERV free modified live PCV1-2 chimeric whole virus vaccines prepared as described herein is that younger pigs (e.g., as early as 1-3 days of age) can be vaccinated, although the present invention is not limited by the age of the pig. In addition, an immune response elicited in an animal in response to a live antigen typically includes both cellular mediated immunity (CMI) and humoral immunity, thus a booster (i.e., second dose) may not be needed.

In one embodiment, at least one additional antigen which can be added to a PCV1-2d chimeric virus prepared as described herein is selected from the following: another chimeric porcine circovirus such as PCV1-2a or PCV1-2b, a wild type PCV2a or PCV2b strain, porcine reproductive and respiratory syndrome virus (PRRSV), such as a North American PRRS virus strain, a Chinese PRRS virus strain, or a European PRRS virus strain, Influenza A Virus of Swine (IAV-S), Porcine Epidemic Diarrhea (PED) virus, Lawsonia intracellularis, M. hyopneumoniae or other combinable viruses and bacteria. Also, combinations of these additional antigens may be employed. The additional antigens can in some cases be grown on PERV negative swine cells. However, again, since most swine vaccines on the market today are inactivated and since the inactivation process likewise inactivates any contaminating PERVs, it is not strictly necessary that inactivated swine vaccines containing additional antigens for use in combination with a PERV negative chimeric PCV1-2d vaccine also be PERV free.

In one embodiment, the ORF1 replicase portion of the PCV1-2d chimeric virus described herein corresponds to SEQ ID NO: 18. In another embodiment, the ORF2 capsid gene of the PCV1-2d chimeric virus described herein corresponds to SEQ ID NO: 19. In a still further embodiment, the entire sequence encoding the PCV1-2 chimeric virus described herein corresponds to SEQ ID NO: 16. However, the present invention is not limited to these embodiments. For example, as described above, the ORF2 portion can be derived from other PCV2d strains besides PCV2d, Isolate Z12. Also, the PCV2d ORF2 portion can, in some embodiments, be a variant or a fragment of a PCV2d ORF2 derived from a PCV2d field virus, examples of which include, but are not limited to those with the GenBank accession numbers referred to herein. Any ORF2 fragment should maintain the epitopes on PCV2d required to elicit a protective response in the animal.

In one embodiment, the vaccine is in the form of a modified live chimeric PCV1-2d virus grown on PERV negative swine cells. The modified live chimeric virus is a recombinant modified live porcine circovirus type 1 that comprises and/or expresses a PCV2d ORF2 polypeptide (PERV free modified live chimeric PCV1-2d virus). The present examples demonstrate that a modified live PCV1-2d chimeric virus-based vaccine which was grown on PERV negative swine cells is suitable for use in neonatal pigs to overcome maternally derived antibodies without the need for a booster vaccination. In addition, since the PCV1-2d virus is PERV free, it eliminates the potential for PERV transmission to the humans administering the vaccine. In addition, vaccines could be produced to keep PERV free pigs intended for xenotransplantation safe from infections with swine pathogens, including PCV2, while simultaneously keeping them PERV free.

In another embodiment, the composition is in the form of an inactivated version of the PERV free chimeric virus, wherein the chimeric virus comprises an inactivated recombinant porcine circovirus type 1 that comprises and/or expresses a PCV2d ORF2 polypeptide (PERV free inactivated chimeric PCV1-2d virus). For example, it may be desirable to combine an inactivated version of the PERV free PCV1-2d virus with inactivated vaccines directed against the same or different swine pathogens to arrive at a multivalent vaccine.

Alternatively, the modified live chimeric PCV1-2d virus can be combined with one or more other modified live PERV free vaccines. An example includes, but is not limited to, a PERV free modified live PRRS virus vaccine that has been grown on PERV negative swine cells that have been engineered to express porcine CD163. One or more liquids can be used to rehydrate these vaccines since they are typically in lyophilized form. It is envisioned that the different antigenic components can be present in lyophilized form in the same or in different containers. If present in the same container, a single fluid can be used to rehydrate the lyophilized material to arrive at a modified live multivalent vaccine composition suitable for administration to pigs, including neonatal pigs. If the different antigenic components are present in different containers, then it is envisioned that the same or different liquids can be used to individually rehydrate the lyophilized antigens before they are combined to arrive at the modified live multivalent vaccine.

Chimeric porcine circoviruses and methods for their preparation are described in WO 03/049703 A2, and in U.S. Pat. Nos. 7,279,166, 7,575,752, and 9,987,348, which are incorporated herein by reference in their entirety. Exemplary methods used for preparation of the PERV free modified live PCV1-2d chimeric virus are disclosed in the example section, together with suitable inactivation methods if inactivation of the PERV free PCV1-2d vaccine is desired.

In some forms, immunogenic portions of PCV2d ORF2 protein are used as the antigenic component in the PCV1-2d vaccine composition. For example, truncated and/or substituted forms or fragments of PCV2d ORF2 protein may be employed in the compositions of the present invention.

It is understood by those of skill in the art that variants of the of the PERV free PCV1-2d chimeric whole virus can be employed in the compositions of the present invention, provided they still retain the antigenic characteristics that render it useful in the vaccine compositions of this invention. For example, in some embodiments, the PCV2d ORF2 capsid polypeptide, which is encoded by the PERV free chimeric PCV1-2d and bears the signature ORF2 amino acid residues of the PCV2d genotype discussed above will have at least 80%, preferably at least 85%, more preferably at least 90%, even more preferably at least 95% sequence identity with the ORF2d capsid sequence of SEQ ID NO: 20. The antigenic characteristics of an immunological composition can be, for example, estimated by the challenge experiment as provided in the Examples. Moreover, the antigenic characteristic of a modified PCV2d ORF2 antigen is still retained when the modified antigen confers at least 70%, preferably 80%, more preferably 90% of the protective immunity as compared to the wild-type PCV2d ORF2 protein having the amino acid sequence of SEQ ID NO: 20.

The PCV1-2d antigen component is provided in the immunogenic/vaccine composition at an antigen inclusion level effective for inducing the desired immune response, namely reducing the incidence of, or lessening the severity of clinical signs resulting from infection with a virulent PCV2d strain, an example of which is the virus termed PCV2d, Isolate Z12 herein. In some embodiments, the composition also provides heterologous protection against classical PCV2a and PCV2b strains.

In one embodiment, a PERV free vaccine composition according to the present invention is in the form of a modified live or inactivated recombinant porcine circovirus type 1 that comprises and/or expresses a PCV2d ORF2 polypeptide (chimeric PCV1-2d virus). This chimeric live virus is included in the compositions of the invention at a level of at least 2.8 log (10) TCID (50)/Dose, for modified live vaccines. This chimeric virus is included in the compositions of the invention at a level of at least 1.0 RP (where 1.0 RP is determined to be between 0.4 and 50 ug/ml of PCV2d ORF 2 polypeptide), for inactivated vaccines, wherein RP is the Relative Potency unit determined by ELISA antigen quantification (in vitro potency test) compared to a reference vaccine with antigen concentrations demonstrated to elicit the desired protective immune response in pigs In another embodiment, a chimeric PCV1-2d virus is included in the composition of the invention at a final concentration of about 0.5% to about 70% of up to 50-times (50×) concentrated bulk chimeric PCV1-2d antigen.

In one embodiment, the chimeric PCV1-2d content of the modified live finished product can be determined by live viral titration using a mouse monoclonal antibody specific for detection of PCV2d (primary antibody) and a commercial fluorometric antibody (secondary antibody, e.g., Jackson ImmunoResearch #115-545-003) for detection of virus in infected cells using fluorescent light. In a further embodiment, the chimeric PCV1-2d content of the inactivated finished product can be determined by ELISA using a mouse monoclonal antibody specific for PCV2d as capture antibody and a biotinylated mouse monoclonal PCV2 antibody and commercial streptavidin peroxidase for detection (e.g., Jackson ImmunoResearch #016-030-084). If the finished vaccine product is a monovalent vaccine comprising only the chimeric PCV1-2d as the antigen, then the reference vaccine is containing only chimeric PCV1-2d antigen. A placebo vaccine without chimeric PCV1-2d antigen and a positive control vaccine batch are used as controls.

In one embodiment, the PCV2d ORF2 protein coat of the virus can be included in the compositions of the invention of inactivated vaccine at a level of at least 0.2 ug antigen/ml of the final immunogenic/vaccine composition (ug/ml). In a further embodiment, the PCV2d ORF2 polypeptide inclusion level is from about 0.2 to about 400 μg/ml. In yet another embodiment, the recombinant PCV2d ORF2 polypeptide inclusion level is from about 0.3 to about 200 μg/ml. In a still further embodiment, the PCV2d ORF2 polypeptide inclusion level is from about 0.35 to about 100 μg/ml. In still another embodiment, the PCV2d ORF2 polypeptide inclusion level is from about 0.4 to about 50 g/ml.

In one embodiment, a vaccine composition of the present invention includes the combination of a PERV free PCV1-2d chimeric virus, and at least one M. hyopneumoniae protein antigen (e.g., two or more). In one embodiment, a vaccine composition of the invention includes a PCV2d ORF2 polypeptide and one or more of the following M. hyopneumoniae-specific protein antigens: M. hyopneumoniae proteins of approximately 46 kD (p46), 64 kD (p64) and 97 kD (p97) molecular weights. The M. hyopneumoniae protein of approximately 64 kD (p64) may be alternatively referred to as the p65 surface antigen from M. hyopneumoniae described by Kim et al. [Infect. Immun. 58 (8): 2637-2643 (1990)], as well as in U.S. Pat. No. 5,788,962. Futo et al. described the cloning and characterization of a 46 kD surface protein from M. hyopneumoniae, which can be employed in the compositions of this invention [J. Bact 177:1915-1917 (1995)]. Zhang et al. described and characterized a p97 adhesin protein of M. hyopneumoniae [Infect. Immun. 63:1013-1019, 1995]. Additionally, King et al. described a 124 kD protein termed Mhp1 from the P-5722 strain of M. hyopneumoniae and presented data suggesting that Mhp1 and p97 are the same protein [Vaccine 15:25-35 (1997)]. Such p97 proteins can be employed in the compositions of this invention. Vaccine compositions of the present invention may include further M. hyopneumoniae specific protein antigens such as, but not limited to, proteins of approximately 41 kD (p41), 42 kD (p42), 89 kD (p89), and 65 kD (p65). See, Okada et al., 2000, J. Vet. Med. B 47:527-533 and Kim et al., 1990, Infect. Immun. 58 (8): 2637-2643. In addition, the M. hyopneumoniae component can include M. hyopneumoniae specific protein antigens of approximately 102 kD (p102) and 216 kD (p216). See, U.S. Pat. Nos. 6,162,435 and 7,419,806 to Minion et al.

The M. hyopneumoniae content of the finished product can be determined by ELISA using a monoclonal antibody specific for the p46 protein content of M. hyopneumoniae pneumoniae and a monoclonal antibody labelled with peroxidase for detection. In one embodiment, M. hyopneumoniae p46 antigen is included at a final concentration of about 1.5 μg/ml to about 10 μg/ml, preferably at about 2 μg/ml to about 6 μg/ml.

In another embodiment, a vaccine composition of the present invention includes the combination of a PERV free PCV1-2d chimeric virus, at least one M. hyopneumoniae protein antigen (e.g., two or more), as well as a PRRS virus antigen. Suitable PRRS virus antigens for use in PCV1-2d/M. hyopneumoniae/PRRS compositions of the present invention include North American PRRS virus isolates, Chinese PRRS virus strains, and European PRRS virus strains, as well as genetically modified versions of such isolates/strains. In one embodiment, the PRRS virus antigen component employed in the compositions according to the present invention is a North American PRRS virus. Preferably, the genetically modified PRRS virus is unable to produce a pathogenic infection yet is able to elicit an effective immunoprotective response against infection by the wild-type PRRS virus.

In some embodiments, the PRRS virus antigen component employed in the compositions of this invention is the North American PRRS virus isolate designated P129 or a live, genetically modified version thereof.

A genetically modified PRRS virus for use in the compositions of the invention can be produced from an infectious clone. The preparation of an infectious cDNA clone of the North American PRRS virus isolate designated P129 is described in U.S. Pat. No. 6,500,662 which is hereby incorporated fully by reference. The sequence of P129 cDNA is disclosed in Genbank Accession Number AF494042 and in U.S. Pat. No. 6,500,662.

In one embodiment, a combination vaccine including a PERV free modified live PCV1-2d chimeric virus prepared as described herein and at least one M. hyopneumoniae protein antigen is provided as a single-dose, 2-bottle vaccine. For example, in some embodiments, a modified live PERV free PCV1-2d vaccine is provided in a lyophilized state in one bottle and an M. hyopneumoniae vaccine including one or more M. hyopneumoniae protein antigens is provided as a stable liquid composition in another bottle, wherein the M. hyopneumoniae liquid composition can be used to rehydrate the lyophilized PERV free modified live PCV1-2d virus so that both vaccines can be administered to the animal in a single dose. It is also possible that both antigens can be included in the same bottle, particularly if both antigenic fractions are inactivated forms and if the prospect of interference between the PERV free PCV1-2 antigenic component and the M. hyopneumoniae antigenic component has been addressed. For example, in one embodiment, the M. hyopneumoniae antigenic component is the soluble portion of an M. hyopneumoniae whole cell preparation which has been treated with Protein A or G to remove anti-PCV2 antibodies (derived from the swine serum used in the M. hyopneumoniae culture media) which would otherwise interfere with the PCV2 antigen (i.e., the PERV free PCV1-2d antigen). Such an M. hyopneumoniae soluble portion is described for example in U.S. pat. No. 9,12,885, the disclosure of which is incorporated herein by reference in its entirety. In some embodiments, additional porcine antigens can be added to either the first or the second bottle.

Methods of making viral or obligate intracellular bacterial antigens according to the methods of the present invention which are suitable for inclusion in the vaccine compositions disclosed herein will now be described in some further detail. For example, the viruses or obligate intracellular bacteria can be propagated in the PERV negative SKB7 cells disclosed herein in tissue culture flasks, shaker flasks, bioreactors, wave bags or other vessels commonly known in the art. The cells may be propagated in suspension or by using treated vehicles for cell attachment and/or can be adapted to environments that contain low oxygen concentrations (microaerophilic conditions) by methods which are readily known and accessible to those of ordinary skill in the art. Said viruses or obligate intracellular bacteria that are capable of being grown/propagated on the PERV negative SKB7 cells can be attenuated or inactivated prior to use in an immunogenic composition or vaccine.

Methods of making bacterial antigens which do not require a cell line to support their propagation, but which are still suitable for inclusion in the vaccine compositions disclosed herein will now be described in some further detail. For example, the bacteria can be propagated in shaker flasks, fermentors, bioreactors, wave bags or other vessels commonly known in the art. The bacteria may be propagated in suspension or by using treated vehicles for attachment and/or can be adapted to environments that contain low oxygen concentrations (microaerophilic conditions) by methods which are readily known and accessible to those of ordinary skill in the art. Said bacteria can be attenuated or inactivated prior to use in an immunogenic composition or vaccine.

Methods of attenuation and inactivation are well known to those skilled in the art. Methods for attenuation include, but are not limited to, serial passage, ultraviolet irradiation, and chemical mutagenesis. Preferred inactivation methods include, but are not limited to, treatment with formalin, betapropriolactone (BPL) or binary ethyleneimine (BEI), or other chemical and/or physical methods known to those skilled in the art. Inactivation by formalin can be performed by mixing the virus or bacteria suspension with 37% formaldehyde to a final formaldehyde concentration of 0.05%. The virus-formaldehyde mixture or bacteria-formaldehyde mixture is mixed by constant stirring for approximately 24 hours at room temperature. The inactivated virus or bacteria mixture is then tested for residual live virus or obligate intracellular bacteria by assaying for growth on a suitable cell line or for residual live bacteria for growth on a suitable media.

Inactivation by BEI can be performed by mixing the virus or bacteria suspension of the present invention with 0.1 M BEI (2-bromo-ethylamine in 0.175 N NaOH) to a final BEI concentration of 1 mM. The virus-BEI mixture or bacteria-BEI mixture is mixed by constant stirring for approximately 48 hours at room temperature, followed by neutralization with the addition of 1.0 M sodium thiosulfate to a final concentration of 0.1 mM. Mixing is continued for an additional two hours. The inactivated virus or bacteria mixture is tested for residual live virus by assaying for growth on a suitably susceptible cell line (for virus or obligate intracellular bacteria) or on suitable media (for bacteria).

Inactivation by Beta-propiolactone (BPL) can be performed by the addition of BPL to the viral or bacterial material to obtain for example a concentration of 0.2% v/v. The viral or bacterial fluids are then agitated for a minimum of 15 minutes at room temperature followed by additional agitation at 2-7° C., for a minimum of 24 hours. After a minimum of 24 hours, a second addition of 0.2% v/v of BPL could be added to the viral or bacterial suspension and maintained at 2-7° C., with constant agitation, for an additional time of not less than 84 hours. In general, the total inactivation time is not less than 108 hours and not more than 120 hours.

Other preferred chemical inactivation agents comprise but are not limited to Triton X-100, Sodium deoxycholate, Cetyltrimethylammonium bromide, Thimerosal, and Phenol. The inactivation may also comprise a neutralization step. Preferred neutralization agents include but are not limited to sodium thiosulfate, sodium bisulfite and the alike.

Purified viruses, such as including but not limited to the PERV free PCV1-2d chimeric virus described herein can be used directly in an immunogenic composition or vaccine, or can be further attenuated, or inactivated. Typically, an immunogenic composition or vaccine contains between about 1×10\hat{ }2 and about 1×10\hat{ }12 virus particles, or between about 1 ×10\hat{ }3 and about 1×10\hat{ }11 virus particles, or between about 1×10\hat{ }4 and about 1×10\hat{ }10 virus particles, or between about 1×10\hat{ }1 and about 1×10\hat{ }9 virus particles, or between about 1×10\hat{ }6 and about 1×10\hat{ }8 virus particles. The precise amount of a virus in an immunogenic composition or vaccine effective to provide a protective effect can be determined by a skilled artisan. Antigen components may also contain membrane fractions or purified immunogenic proteins of the viruses disclosed above. Methods of fractionation and/or purification and/or recombinant techniques for production of the immunogenic proteins are well known in the art. The antigen component may comprise a vectored vaccine containing a vector (e.g., a viral vector such as, for example, a canarypox vector, an adenovirus vector or a vaccinia vector) carrying a nucleic acid sequence encoding an immunogenic protein capable of eliciting protective immune response to a challenge with virus strain(s) or types disclosed herein.

The antigen component may comprise a live or non-replicating vectored vaccine containing a vector (e.g., a viral vector such as, for example, a canarypox vector, an adenovirus vector or a vaccinia vector or a bacterial vector such as, for example, Salmonella or E. coli) carrying a nucleic acid sequence encoding an immunogenic protein capable of eliciting protective immune response to a strain(s) or types disclosed herein.

The antigen component may be either a chimeric virus, such as a PCV1-2 chimeric virus (which is part PCV1 and part PCV2), a single virus, such as BVDV Type 1, or a combination of viruses of a similar type, such as BVDV Type I and BVDV Type 2 or a combination of viruses of differing types, such as PCV2 and AIV-S, or an obligate intracellular bacteria such as Lawsonia intracellularis. Multivalent combinations of viruses and bacteria such as PCV2, AIV-S and PRRS or PCV2, PRRS and Lawsonia intracellularis or PCV2, PRRS and M. hyopneumoniae may also be considered in the present invention.

Viral and bacterial vaccine compositions made in accordance with the method of the present invention can include one or more veterinary or pharmaceutically acceptable carriers. These carriers include any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others. Stabilizers include albumin, among others.

Vaccines made according to the method of the present invention can further include one or more additional immunomodulatory components such as, e.g., an adjuvant, cytokine, or interferon, among others. Types of suitable adjuvants for use in the compositions of the present invention include the following: an oil-in-water emulsion, a polymer and water adjuvant, a water-in-oil emulsion such as, e.g., Freund's complete and incomplete adjuvants, alum, an aluminum hydroxide adjuvant, a vitamin E adjuvant and combinations thereof. Some specific examples of adjuvants include, but are not limited to, complete Freund's adjuvant, incomplete Freund's adjuvant, Corynebacterium parvum, Bacillus Calmette Guerin, aluminum hydroxide gel, glucan, dextran sulfate, iron oxide, sodium alginate, Bacto-Adjuvant, certain synthetic polymers such as poly amino acids and co-polymers of amino acids, Block copolymer (CytRx, Atlanta, GA.), QS-21 (Cambridge Biotech Inc., Cambridge MA), SAF-M (Chiron, Emeryville CA), AMPHIGEN® adjuvant, saponin, Quil A, GPI-0100 (Galencia Pharmaceuticals, Inc., Birmingham, AL) or other saponin fractions, monophosphoryl lipid A, and Avridine lipid-amine adjuvant (N,N-dioctadecyl-N′,N′—-bis(2-hydroxyethyl)-propanediamine), “REGRESSIN” (Vetrepharm, Athens, GA), paraffin oil, RIBI adjuvant system (Ribi Inc., Hamilton, Mont.), cholesterol, heat-labile enterotoxin from E. coli (recombinant or otherwise), cholera toxin, or muramyl dipeptide, among many others.

Another example of an adjuvant useful in the compositions of the invention is SP-oil. As used in the specification and claims, the term “SP oil” designates an oil emulsion comprising a polyoxyethylene-polyoxypropylene block copolymer, squalane, polyoxyethylene sorbitan monooleate and a buffered salt solution. Polyoxyethylene-polyoxypropylene block copolymers are surfactants that aid in suspending solid and liquid components. These surfactants are commercially available as polymers under the trade name Pluronic®. The preferred surfactant is poloxamer 401 which is commercially available under the trade name Pluronic® L-121. In general, the SP oil emulsion is an immunostimulating adjuvant mixture which will comprise about 1 to 3% vol/vol of block copolymer, about 2 to 6% vol/vol of squalane, more particularly about 3 to 6% of squalane, and about 0.1 to 0.5% vol/vol of polyoxyethylene sorbitan monooleate, with the remainder being a buffered salt solution. In one embodiment, the SP-oil emulsion is present in the final composition in v/v amounts of about 1% to 25%, preferably about 2% to 15%, more preferably about 5% to 12% v/v.

Other examples of adjuvants useful in the compositions of the invention are the following proprietary adjuvants: Microsol Diluvac Forte® duel emulsion adjuvant system, Emunade adjuvant, and Xsolve adjuvant. Both the Emunade and Xsolve adjuvants are emulsions of light mineral oil in water, but Emunade also contains alhydrogel, and d,I-α-tocopheryl acetate is part of the XSolve adjuvant. A still further example of a suitable adjuvant for use in the compositions of the invention is ImpranFLEX™ adjuvant (a water-in-oil adjuvant). A still further example of a suitable adjuvant is a Carbomer (Carbopol®) based adjuvant. Preferred Carbopol® adjuvants include Carbopol® 934 polymer and Carbopol®941 polymer.

In one embodiment, the adjuvant or adjuvant mixture is added in an amount of about 100 μg to about 10 mg per dose. In another embodiment, the adjuvant/adjuvant mixture is added in an amount of about 200 μg to about 5 mg per dose. In yet another embodiment, the adjuvant/adjuvant mixture is added in an amount of about 300 μg to about 1 mg/dose.

The adjuvant or adjuvant mixture is typically present in the vaccine composition of the invention in v/v amounts of about 1% to 25%, preferably about 2% to 15%, more preferably about 5% to 12% v/v.

The vaccine compositions can also include other additives such as antibiotics or preservatives. By way of example only, Gentamicin and Merthiolate can be added.

While the amounts and concentrations of adjuvants and additives useful in the context of the present invention can readily be determined by the skilled artisan, the present invention contemplates compositions comprising from about 10 μg to about 2000 μg of adjuvant and preferably about 0.5 mL to 2 mL dose of the vaccine composition. In another preferred embodiment, the present invention contemplates vaccine compositions comprising from about 1 μg/ml to about 60 μg/ml of antibiotic, and more preferably less than about 30 μg/ml of antibiotic.

The immunogenic compositions of the present invention can be made in various forms depending upon the route of administration. For example, the immunogenic compositions can be made in the form of sterile aqueous solutions or dispersions suitable for injectable use or made in lyophilized forms using freeze-drying techniques. Lyophilized immunogenic compositions are typically maintained at about 4° C., and can be reconstituted in a stabilizing solution, e.g., saline or and HEPES, with or without adjuvant.

Vaccine compositions according to the present invention can be administered in dosages and by techniques well known to those skilled in the medical or veterinary arts, taking into consideration such factors as the age, sex, weight, species and condition of the recipient animal, and the route of administration. The route of administration can be percutaneous, via mucosal administration (e.g., oral, nasal, anal, vaginal) or via a parenteral route (intradermal, transdermal, intramuscular, subcutaneous, intravenous, or intraperitoneal). Vaccine compositions according to the present invention can be administered alone, or can be co-administered or sequentially administered with other treatments or therapies. Forms of administration may include suspensions, syrups or elixirs, and preparations for parenteral, subcutaneous, intradermal, intramuscular or intravenous administration (e.g., injectable administration), such as sterile suspensions or emulsions. Vaccine compositions according to the present invention may be administered as a spray, or mixed in food and/or water, or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired.

Typical administration routes of viral and bacterial vaccines include, but are not limited to, intramuscular or subcutaneous injection (by needle or needless administration) of between about 0.1 and about 5 mL of vaccine. Other routes of administration can be used as well, such as e.g., by oral, intranasal (e.g. aerosol or other needleless administration), intra-lymph node, intradermal, intraperitoneal, rectal or vaginal administration, or by a combination of routes. Boosting regimens and administration methods may be required and the dosage regimen can be adjusted to provide optimal immunization.

The following examples set forth preferred materials and procedures in accordance with the present invention. However, it is to be understood that these examples are provided by way of illustration only, and nothing therein should be deemed a limitation upon the overall scope of the invention.

### Examples

### Example 1 CRISPR GENE-EDITING OF PK-15 CELL LINE

### Overall Strategy

A knock-out strategy was chosen consisting of targeting the polymerase gene of Porcine Endogenous Retrovirus (PERV) in a PK-15 cell line. The polymerase (pol) gene of PERV codes for reverse transcriptase (RT) activity which is essential for PERV replication. An initial guide RNA (gRNA) was designed to target outside the catalytic region (gRNA 1847) of the PERV pol gene. Cells were sorted based on CRISPR cleavage efficiency, and then subjected to an additional round of gene editing with another gRNA (gRNA 1844) targeting inside the catalytic region of pol. The details regarding how the inventors arrived at this strategy are discussed below.

### Origin of the PK-15 Cell Line

A PK-15 cell line available from the American Type Culture Collection (ATCC accession number CCL-33) was employed as the original cell line from which a PERV negative CRISPR edited cell line was ultimately derived. The PK-15 cell line was received by Fort Dodge Animal Health (FDAH), now part of Zoetis, from Dr. X. J. Meng at Virginia Polytechnic Institute and State University, Blacksburg, Virginia. At FDAH, the PK-15 cells were expanded to passage 4 and frozen. Thereafter, the fourth passage was thawed and further expanded to passage 9. The ninth passage of the PK-15 cells was frozen and established as the Master Cell Stock and is the parental cell line.

The PK-15 cells were grown in 1× Minimum Essential Medium supplemented with 0.05% lactalbumin hydrolysate, 30 μg/mL gentamicin sulfate, and 5% fetal bovine serum. No special grown enhancer was added to this medium for PK-15 cell propagation. The Master Cell Stock was thawed and then further expanded 7 times to generate a working cell stock (WCS) which was sent to Horizon Discovery Ltd, Cambridge, United Kingdom, for gene editing using CRISPR/Cas9 gene to remove the PERV activity in the PK-15 cell line.

Evaluation of PK-15 parental Cell Line-preparation for Gene Editing

The PK-15 cells which Horizon Discovery Ltd obtained from Zoetis were further expanded, characterized, and used to optimize CRISPR reagents and processes in preparation for gene editing.

### Characterization of the Cells

The PK-15 cells (referred to as the parental cell) were analyzed and optimized for transfection efficiency by both electroporation and lipid transfection, single cell cloning in 384 well plates, CRISPR reagent delivery, and Florescence-Activated Cell Sorting (FACS). It was determined that PK-15 cells can be transfected by electroporation at a rate of 84.8% with 73.6% cell viability and that single cell PK-15 clones can be recovered at a rate of 20% and successfully expanded into 12 well plates. In addition, Florescence-activated Cell Sorting (FACS) was confirmed as a suitable method for segregation of the cells edited by transfection with fluorescently labeled Cas9 nuclease mRNA. This method was used for enrichment of the targeted cells following each transfection replicate.

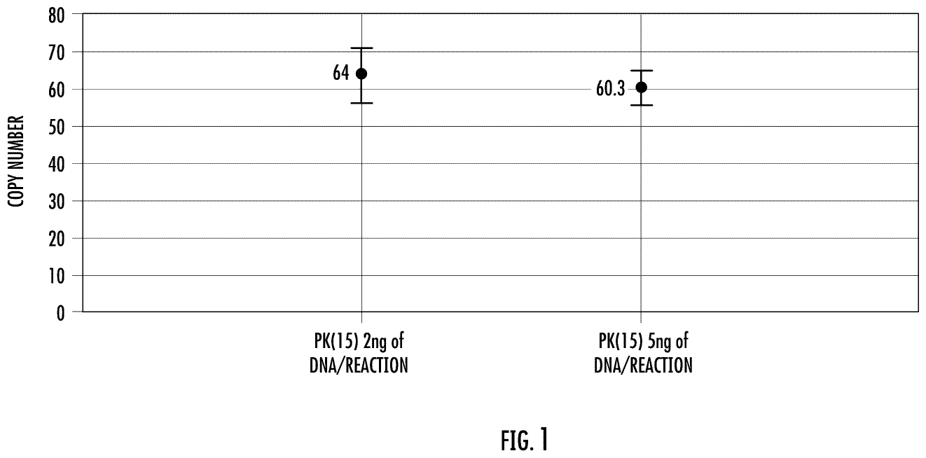

Digital droplet PCR (ddPCR) indicated that approximately 62 copies of the PERV locus were present in the PK-15 parental cell line, based on a copy number of 2 for the reference gene (assumption based on the diploid karyotype of the cell line)—FIG. 1. This was consistent with other reports demonstrating integration across the genome (Yang et al., Science 350:1101-4, 2015).

### Selection of Guide RNA for Inactivation of PERV

A consensus PERV polymerase gene sequence was generated that was derived from an alignment of 16 published PERV polymerase sequences from PERV-A, PERV-B and PERV-C. This 3441 bp sequence was used for reference to design target editing guide RNAs (gRNAs). The sequences included in the alignment have the following NCBI GenBank accession numbers. HQ536009.1, Y17013.1, AY099323.1, AY099324.1, HQ536008.1, HQ536010.1, HQ540593.1, HQ540594.1, HQ540595.1, AJ133816.1, AJ133817.1, AJ133818.1, AJ293657.1, AJ279057.1, AY056035.1, and EU523109.1. The consensus PERV sequence which is represented by SEQ ID NO: 1 was sent from Zoetis to Horizon Discovery Ltd.

### Genotyping Strategy-qDNA

Amplification and sequencing primers were designed in conserved regions of the PERV polymerase (pol) sequence of SEQ ID NO: 1. The polymerase region of PERV was amplified and sequenced to identify regions for editing. Based on the conserved PERV sequence, eight guide RNA sequences (gRNAs) were designed to target the polymerase region, either adjacent to or within the conserved catalytic sequence. Two of these eight gRNAs were the same as those previously disclosed by Yang et al. (Yang et al., Science 350:1101-4, 2015). These were chosen for comparison as Yang et al. previously demonstrated successful pol gene editing with these gRNA sequences. The gRNAs (20 bp), as disclosed by Yang et al., are shown below. These were renamed as gRNA 1848 and gRNA1849 in the studies described herein.

| | gRNA1/gRNA1848: |
|---|---|
| (SEQ ID NO: 2) | |
| | 5′ GGTCATCCACGTACTGGAGG 3′ |
| | gRNA2/gRNA1849: |
| (SEQ ID NO: 3) | |
| | 5′ GGTGACCCTCCTCCAGTACG 3′ |

The eight gRNAs were individually transfected using Dharmacon™ Edit-R fluorescent (GFP) Cas9 nuclease mRNA and Dharmacon™ Edit-R synthetic crRNA: trRNA complexes. Pools of cells transfected with individual gRNAs were harvested for analysis 24 hours post transfection. TIDE (Tracking of Indels by Decomposition) analysis was used to assess the efficiency of each gRNA to direct Cas9 nuclease activity to the target loci. A higher percentage of directed Cas9 nuclease activity for an individual gRNA will increase the likelihood of all copies of PERV being disrupted in an individual targeted cell. Cas9 nuclease activity ranged from 4.8 to 43.3% for the individual guides, with the Yang et al. gRNAs performing at the lower range (4.8% and 7.5%). These results are presented below in Table 1 below, where gRNA1 and gRNA2, as described by Yang et al. were renamed as gRNA1848 and gRNA1849, respectively.

TABLE 1
gRNA validation: Cas9 nuclease activity assessment using TIDE

| | | Cas9 nuclease |
|---|---|---|
| RNA ID | Genomic location | activity (%) |
| 1844 | PERV polymerase | 43.3 |
| | sequence | |
| 1845 | PERV pol Catalytic region | 39.1 |
| 1846 | PERV pol Catalytic region | 7.1 |
| 1847 | PERV pol Catalytic region | 12.1 |
| 1848 (Yang L., et al.) | PERV pol Catalytic region | 7.5 |
| 1849 (Yang L., et al.) | PERV pol Catalytic region | 4.8 |
| 1850 | PERV pol Catalytic region | 11.5 |
| 1851 | PERV pol Catalytic region | 5.9 |

Although a higher percentage of directed Cas9 nuclease activity for an individual gRNA was one factor which can influence the likelihood of generating a functional PERV knock-out in an individual cell and was considered, additional factors were also considered. For example, when selecting which of the eight gRNAs to move forward, the efficiency of cutting at the target loci, location of the specific gRNA within the PERV genomic sequence and the potential of the gRNA to create mismatches to gRNA target sequence were all considered. Table 2 below presents the benefits and limitations of four gRNAs which were considered possibly suitable for use in generating a functional PERV knockout.

TABLE 2
Properties of gRNAs Considered for Use in
Generating PERV Knockout

| | | | Cas9 | | Mismatches |
|---|---|---|---|---|---|
| gRNA | | | nuclease | Position of | to PERV |
| ID | Sequence (5′-3′) | PAM | activity | gRNA | sub-variants |
| 1844 | ACCAGTACAGGACTTGAGAG | AGG | High | Upstream | No |
| | (SEQ ID NO: 4) | | | of catalytic | mismatches |
| | | | | region | to high-copy |
| | | | | | number |
| | | | | | subvariants |
| 1845 | CTTGAACCCTTGGGGCAGTC | GGG | High | Within | Terminal 5′ |
| | (SEQ ID NO: 5) | | | catalytic | base |
| | | | | region | has mis- |
| | | | | | match to ca. |
| | | | | | 30% of PERV |
| | | | | | variants |
| 1847 | TACTGGAGGAGGGTCACCTG | AGG | Low- | Adjacent to | No |
| | (SEQ ID NO: 6) | | medium | gRNAs | mismatches |
| | | | | from Yang | to high-copy |
| | | | | et al. | number |
| | | | | | subvariants |
| 1850 | TCTGGCGGGAGCCACCAAAC | AGG | Low- | Adjacent to | No |
| | (SEQ ID NO: 7) | | medium | gRNAs | mismatches |
| | | | | from Yang | to high-copy |
| | | | | et al. | number |

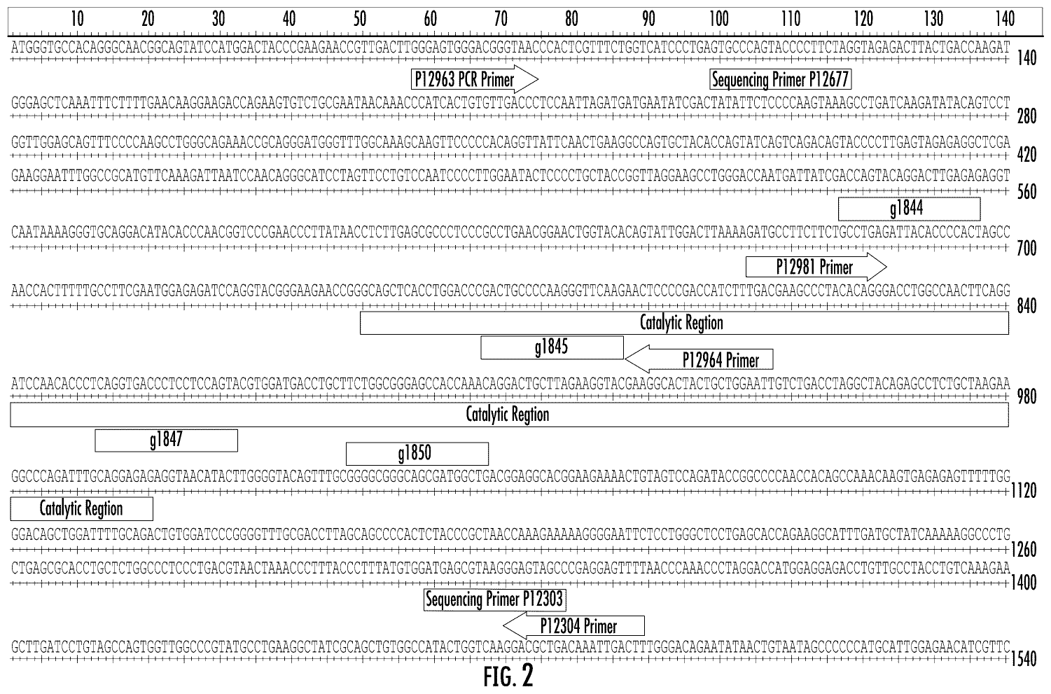

In Table 2 above, gRNAs 1844, 1845, 1847, and 1850 correspond respectively to SEQ ID Nos. 4, 5, 6, and 7. The location of the catalytic region within the consensus PERV polymerase gene sequence of SEQ ID NO: 1, the target sequences of candidate gRNAs 1844, 1845, 1847, and 1850, and the template sequences to which the primers used for amplification and sequencing bind are depicted in FIG. 2. The two gRNA candidates which were ultimately selected for inactivation of the PERV polymerase sequence in the PK15 parental cell line were gRNA1844 (43.3% Cas 9 activity) and gRNA1847 (12.1% Cas 9 activity), neither of which demonstrated mismatches to PERV sub-variants. Both g1844 and g1847 demonstrated higher Cas9 nuclease activity than the Yang et al. gRNAs. The target for guide RNA 1844 is upstream of the catalytic region, at approximately 537 base pairs of the 3441 bp polymerase gene. See FIG. 2. Disruptions in the g1844 region (537 to 556 bp) and subsequent insertions and deletions (indels) that result from NHEJ repair could result in frame-shift mutations and allow for an early stop codon and non-expression of the gene at the protein level. The target for guide 1847 is at approximately 853 base pairs in the pol gene and is located in the catalytic domain of the protein. See FIG. 2. In addition to the potential of NHEJ repair resulting in indels that cause frame-shifts and early stop codons, it is likely that an in-frame edit would also lead to non-functionality of the target protein due to insertion or deletions of amino acids within the essential catalytic region.

To avoid potential introduction of foreign DNA or AMR genes associated with plasmids, the approach used for generating the PERV edited cell line utilized co-electroporation of only the gRNA targeting sequence and mRNA expressing Cas9. The Cas9 mRNA used was Dharmacon's Edit-R fluorescent (GFP) Cas9 nuclease mRNA. The crRNA: tracrRNA sequences were formed using Edit-R synthetic crRNA (gRNA1844 or gRNA1847), which specifically target the PERV polymerase gene (encoding reverse transcriptase activity) and Edit-R synthetic tracrRNA as supplied by Dharmacon. These RNA's were introduced into the cells for the editing reaction. The resulting cells have only a specifically modified target region. Accordingly, there is no inserted vector or foreign DNA but instead there is disruption of the PERV polymerase gene at the targeted regions (gRNA1844 and/or gRNA1847 binding sites) and the relevant assay is the loss of RT activity in the cell line.

Guide RNAs, gRNA 1844 and gRNA 1847, which were selected for final editing of the PERV polymerase sequence in PK-15 cells are shown in FIG. 3A and FIG. 3B, respectively.

Guide RNA 1844 comprises the sequence: 5′ ACCAGTACAGGACTTGAG 3′ (SEQ ID NO: 4), wherein the gRNA binds to the complement of the site which is underlined with a solid line in the following PERV nucleic acid sequence, and the protospacer adjacent motif (PAM) sequence is underlined with a dashed line:

(SEQ ID NO: 8)
TGATTATCGACCAGTACAGGACTTGAGAGAGGTCAATAAAAGGGTGCAGG
AC

Guide RNA 1847 comprises the sequence: 5′ TACTGGAGGAGGGTCACCTG 3′ (SEQ ID NO: 6), wherein the gRNA binds to the site which is underlined with a solid line in the following PERV nucleic acid sequence, and the protospacer adjacent motif (PAM) sequence is underlined with a dashed line:

(SEQ ID NO: 9)
CAACACCCTCAGGTGACCCTCCTCCAGTACGTGGATGACCTGCTTCTGGC
GG

In FIG. 3A, the forward primer designated as P12963 and the reverse primer designated as P12964 correspond to SEQ ID Nos 10 and 11, respectively. These were used to amplify the A-B target region schematically depicted in FIG. 3A to confirm the expected product size for the allele of 751 bp. The forward primer designated as P12677 (SEQ ID NO: 12) was used to sequence the A-B fragment for comparison with the wild-type sequence. In FIG. 3B, the forward primer designated as P12981 and the reverse primer designated as P12304 correspond to SEQ ID Nos 13 and 14, respectively. These were used to amplify the C-D target region schematically depicted in FIG. 3B to confirm the expected product size for the allele of 686 bp. The reverse primer designated as P12303 (SEQ ID NO: 15), was used to sequence the C-D PCR fragment. Both A-B and C-D PCR fragment sequences were compared with the wild type PERV pol sequence to determine modifications (insertions/deletions) that resulted from CRISPR editing using TIDE (Tracking of Indels by Decomposition) analysis. TIDE analysis provides a rapid and reliable assessment of genome editing experiments of a target locus. Based on the quantitative sequence trace data from two standard capillary sequencing reactions (the edited sequence and wild type sequence) the TIDE software uses a decomposition algorithm which quantifies the editing efficacy and identifies the predominant types of insertions and deletions (indels) in the DNA of a targeted cell pool.

### PERV-Edited Cell Line Generation

The two guide RNAs 1844 and 1847 were transfected sequentially to increase the potential for disruption of the PERV polymerase gene. The first guide RNA to be transfected, g1847, targeted the catalytic region of the pol gene and non-cloned pools of two transfection experiments enriched by FACS sorting were shown to have 40-50% Cas9 cleavage efficiency as assessed by TIDE analysis. The g1847 edited pool was subjected to further editing with g1844, targeting upstream of the pol catalytic region. Cas 9 cleavage efficacy was identified at 80% by TIDE analysis.

Single cell clones were obtained through low density plate out experiments. Nineteen clones were identified via TIDE analysis with a targeting rate of >70% at the gRNA1847 site and >80% at the gRNA1844 site. All 19 clones were taken forward for targeting confirmation at each site. Nine clones with the overall highest targeting rates, as determined by TIDE analysis, were selected for an in-house Reverse Transcriptase Assay. The results are shown in Table 3 below.

TABLE 3
Reverse Transcriptase Negative Clones with Overall Highest Targeting Rates

| Clone | Clone | % Targeting | % Targeting | RT Activity | |
|---|---|---|---|---|---|
| ID | Name | (gRNA 1847) | (gRNA 1844) | (MS2) | Clone Growth Rate |
| 1 | 3B5_1 | 78.0 | 93.1 | Negative | Similar to parental |
| 2 | 3B7_1 | 83.0 | 96.9 | Negative | Similar to parental |
| 3 | 3D1_1 | 85.0 | 92.0 | Negative | Similar to parental |
| 4 | 3G3_1 | 79.5 | 92.5 | Negative | Similar to parental |
| 5 | 1B1_2 | 86.4 | 82.7 | Negative | Slower than parental, |
| | | | | | vacuoles in cytoplasm |
| 6 | 2B5_2 | 78.6 | 91.0 | Negative | Similar to parental |
| 7 | 2D11_2 | 94.2 | 82.8 | Negative | Slower than parental, |
| | | | | | vacuoles in cytoplasm |
| 8 | 4E6_2 | 83.3 | 82.6 | Negative | Slower than parental, |
| | | | | | vacuoles in cytoplasm |
| 9 | 3D8_2 | 91.6 | 91.7 | Negative | Similar to parental |

* The cause of the altered growth properties (clones 5, 7, 8) was not determined. These could be changes due to clonal selection or off-target events from cell editing.

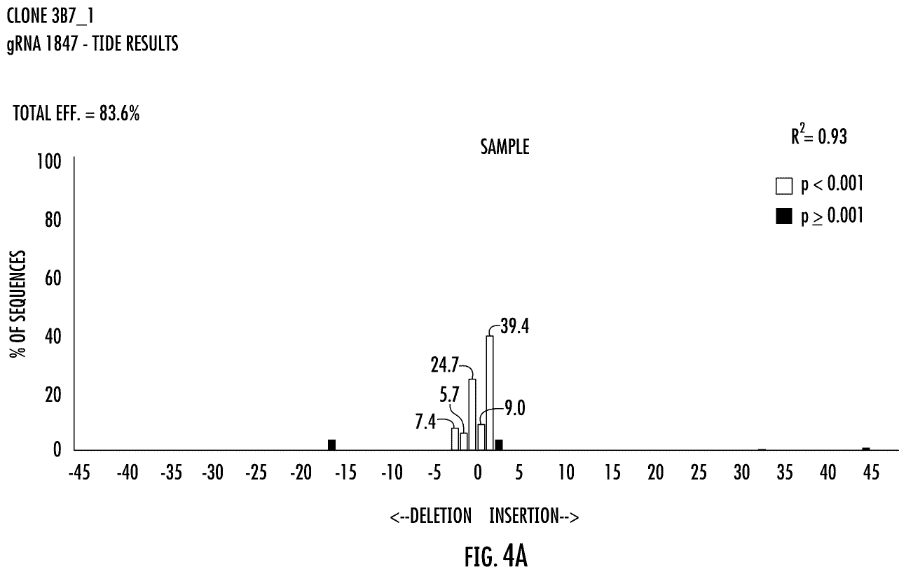

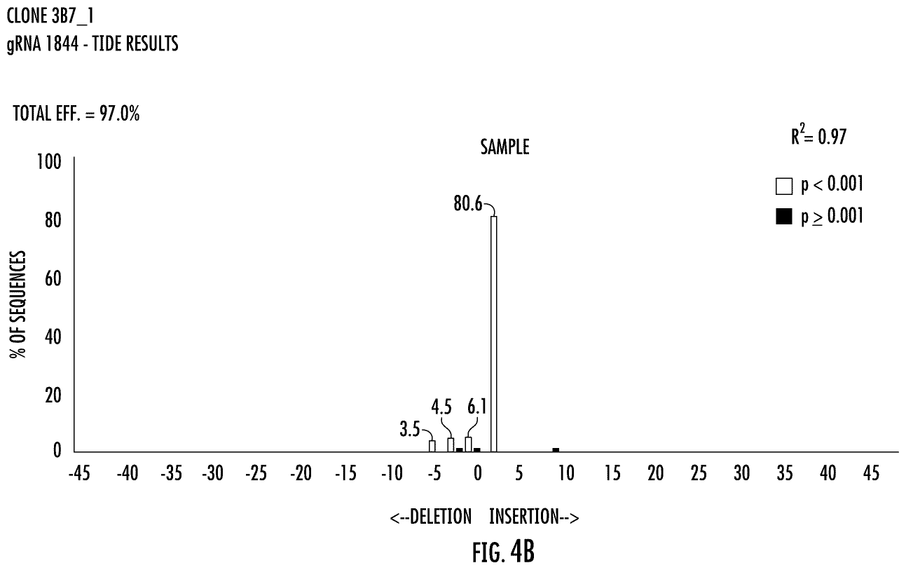

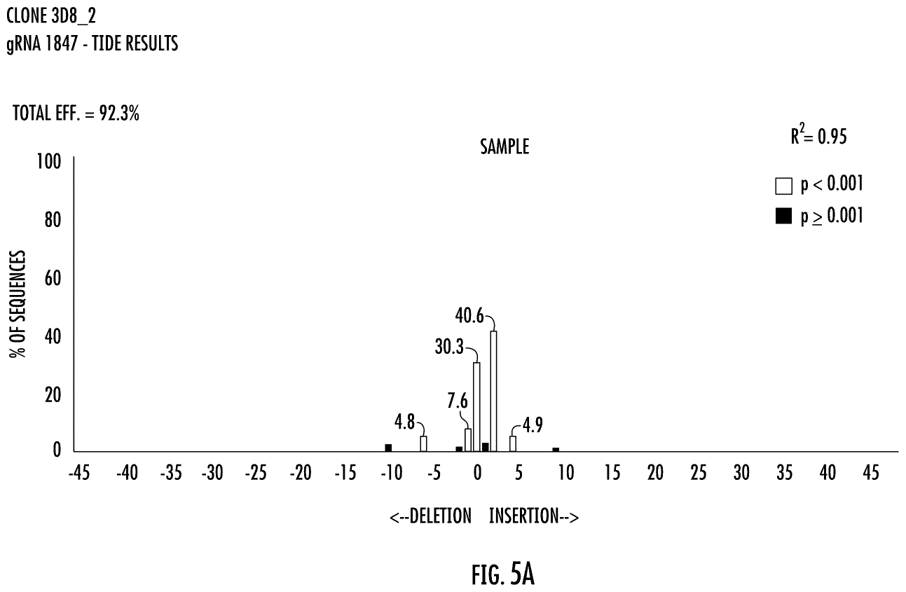

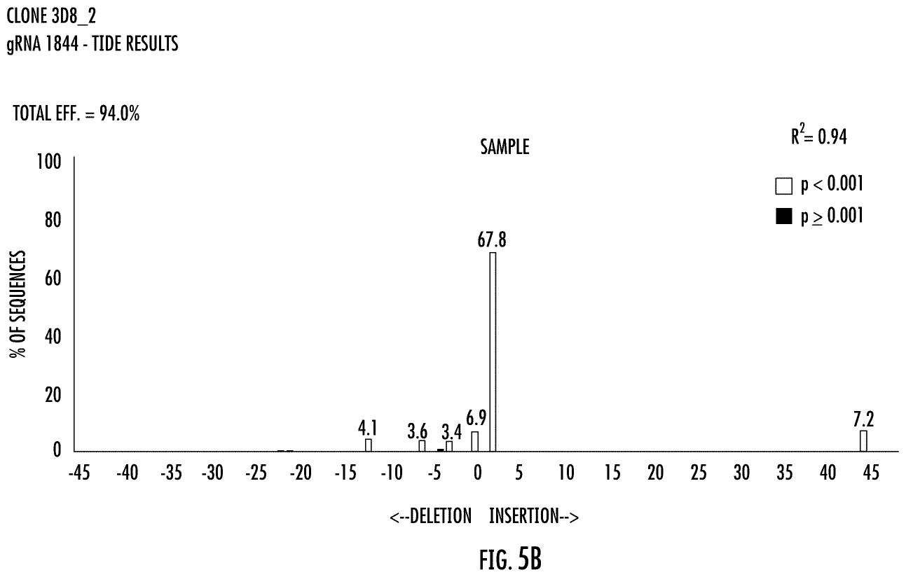

As can be seen from the results in Table 3, all 9 clones were tested negative for Reverse Transcriptase (RT) activity in an MS2 qRT-PCR assay. Of the 9 RT negative clones, 2 were chosen to move forward due to the overall high targeting efficiencies at both targeting loci and growth characteristics similar to the parental PK-15 cells. The two clones chosen to move forward were clones 2 and 9, alternatively referred to herein as clone 3B7_1 and clone 3D8_2, respectively. The TIDE analysis for these two clones is depicted in FIGS. 4A, 4B, 5A and 5B where the different insertion/deletion (indels) size analyzed at the particular gRNA target site are indicated in the X-axis and their distribution represented with histograms.

TIDE analysis of 3B7_1 (SKB7) clone at the g1847 target site is shown in FIG. 4A. TIDE analysis at the gRNA1847 target site showed a targeting efficiency of 83.6% across all the PERV copies and only 9% of the sequence that doesn't contain any indels. All the targeted PERV copies carry out-of-frame or in-frame modifications in the catalytic region that would either create a frame shift within the protein coding sequence and likely generate an early STOP codon or disable the catalytic activity of the retroviral polymerase protein following deletion of essential codons.

TIDE analysis of 3B7_1 (SKB7) at the g1844 target site is shown in FIG. 4B. TIDE analysis at the gRNA1844 target site showed a targeting efficiency of 97% across all the PERV copies. These results appear to indicate that all the PERV copies carry in-frame or out-of-frame modification with 80.6% of them having a 1 bp insertion that creates a frame shift in the protein coding sequence and likely generate and early stop codon. In particular, the gRNA1844 target is near the beginning of the polymerase gene and therefore likely generates out-of-frame (missense) codons and an early STOP codon resulting in efficient inactivation of the retroviral polymerase.

TIDE analysis of Clone 3D8_2 at the g1847 target site is shown in FIG. 5A. TIDE analysis at gRNA1847 target site shows a targeting efficiency of 92.3% across all the PERV copies and less than 5% of the sequence that doesn't contain any indels. All the targeted PERV copies carry out-of-frame or in-frame modifications in the catalytic region that would either create a frame shift within the protein coding sequence and likely generate an early stop codon or disable the catalytic activity of the viral polymerase protein following deletion of essential codons.

TIDE analysis of Clone 3D8_2 at the g1844 target site is shown in FIG. 5B. TIDE analysis at gRNA1844 target site shows a targeting efficiency of 94% across all the PERV copies. The graph seems to indicate that all the PERV copies carry in-frame or out-of-frame modification with 67.8% of them which have a 1 bp insertion that creates a frame shift in the protein coding sequence and likely generate an early stop codon.

The Two clones (clones 2 and 9, alternatively referred to herein as clone 3B7_1 and clone 3D8_2, respectively) with the highest targeting rates and growth properties most similar to parental cells were sent to BioReliance for the PERT-Ultra 107325.BUK Reverse Transcriptase Assay (gold standard) testing. The parental cell line was positive, while clones 2 and 9 were negative. These results are shown in Tables 4A, 4B, and 4C below, where NTC stands for no template control; DMEM stands for Dulbecco's Modified Eagle Medium; MLV stands for Murine Leukemia Virus;—stands for negative which means the mean Ct value was greater than that generated by the 1×1-7 U RT enzyme control; and +stands for positive which means a minimum of 2/3 wells were less than or equal to that generated by the 1×1-7 U RT enzyme control, with amplification greater than or equal to that generated by the 1×1-7 U RT enzyme control.

TABLE 4A
PERT Reverse Transcriptase Testing Results: PK-15 Parental Cell Line

| Sample description | Mean C₁ value | Result | |
|---|---|---|---|
| NTC (nuclease free water) | 39.95 | − | Result PK(15) Parental |
| DMEM control | 34.93 | − | Positive for the presence |
| Test article AF44PB | 23.42 | + | of retroviral reverse |
| Test article AF44PB spiked | 23.96 | + | transcriptase activity. |
| with 10 IU MLV | | | The test article was shown |
| Positive control 10 IU MLV | 27.02 | + | to contain 4.55 × 10⁻⁵ units |
| 10⁻² units RT positive | 14.48 | + | of retroviral reverse |
| control enzyme | | | transcriptase activity per |
| 10⁻³ units RT positive | 18.40 | + | reaction (1.49 × 10⁻³ |
| control enzyme | | | units/ml of test articles) |
| 10⁻⁴ units RT positive | 21.73 | + | |
| control enzyme | | | |
| 10⁻⁵ units RT positive | 27.77 | + | |
| control enzyme | | | |
| 10⁻⁶ units RT positive | 29.17 | + | |
| control enzyme | | | |
| 10⁻⁷ units RT positive | 32.39 | + | |
| control enzyme | | | |

TABLE 4B
PERT Reverse Transcriptase Testing Results: Clone 3B7_1 (aka Clone 2)

| Sample description | Mean C₁ value | Result | |
|---|---|---|---|
| NTC (nuclease free water) | 39.95 | − | Result clone 3B7_1 |
| DMEM control | 34.93 | − | Negative for the |
| Test article AF44PC | 33.23 | − | presence of retroviral |
| Test article AF44PC spiked | 27.46 | + | transcriptase activity |
| with 10 IU MLV | | | |
| Positive control 10 IU MLV | 27.02 | + | |
| 10⁻² units RT positive | 14.48 | + | |
| control enzyme | | | |
| 10⁻³ units RT positive | 18.40 | + | |
| control enzyme | | | |
| 10⁻⁴ units RT positive | 21.73 | + | |
| control enzyme | | | |
| 10⁻⁵ units RT positive | 27.77 | + | |
| control enzyme | | | |
| 10⁻⁶ units RT positive | 29.17 | + | |
| control enzyme | | | |
| 10⁻⁷ units RT positive | 32.39 | + | |
| control enzyme | | | |

TABLE 4C
PERT Reverse Transcriptase Testing Results: Clone 3D8_2 (aka Clone 9)

| Sample description | Mean C₁ value | Result | |
|---|---|---|---|
| NTC (nuclease free water) | 39.95 | − | Result clone 3D8_2 |
| DMEM control | 34.93 | − | Negative for the |
| Test article AF44PD | 33.46 | − | presence of retroviral |
| Test article AF44PD spiked | 27.60 | + | transcriptase activity |
| with 10 IU MLV | | | |
| Positive control 10 IU MLV | 27.02 | + | |
| 10⁻² units RT positive | 14.48 | + | |
| control enzyme | | | |
| 10⁻³ units RT positive | 18.40 | + | |
| control enzyme | | | |
| 10⁻⁴ units RT positive | 21.73 | + | |
| control enzyme | | | |
| 10⁻⁵ units RT positive | 27.77 | + | |
| control enzyme | | | |
| 10⁻⁶ units RT positive | 29.17 | + | |
| control enzyme | | | |
| 10⁻⁷ units RT positive | 32.39 | + | |
| control enzyme | | | |

Following successful testing of clone 3B7_1 and clone 3D8_2, they were renamed SK-B7 and SK-D8 cells, respectively.

### Example 2: Chimeric Porcine Circovirus (cPCV) 1-2d Production Methods

A DNA sequence consisting of the PCV1 ORF-1 (replicase gene) and PCV2d ORF-2 (immunogenic capsid gene) and corresponding to SEQ ID NO: 16 was provided to GenScript USA, Inc. 860 Centennial Ave. Piscataway, NJ 08854, USA for synthesis and cloning. It included an additional repeat of ORF1. The PCV1 ORF-1 replicase portion corresponds to SEQ ID NO: 17 and the PCV2d ORF2 portion corresponds to SEQ ID NO: 18, which encodes the PCV2d ORF2 capsid protein corresponding to SEQ ID NO: 19. The plasmid construct was received at Zoetis, Kalamazoo, MI for preparation of the cPCV1-2d virus. The resultant virus is a completely synthetic chimeric virus whose genome encodes the ORF-1 replicase gene from Porcine Circovirus Type 1 and the ORF-2 immunogenic capsid gene from Porcine Circovirus Type 2d (PCV2d isolate Z12), isolated from a pig from a loss of efficacy case in Oakville, Iowa. The PCV2d isolate Z12 donor portion is 705 bp ORF-2 which encodes a capsid protein which corresponds to GenBank accession #HM03801 (complement region 1030-1734).

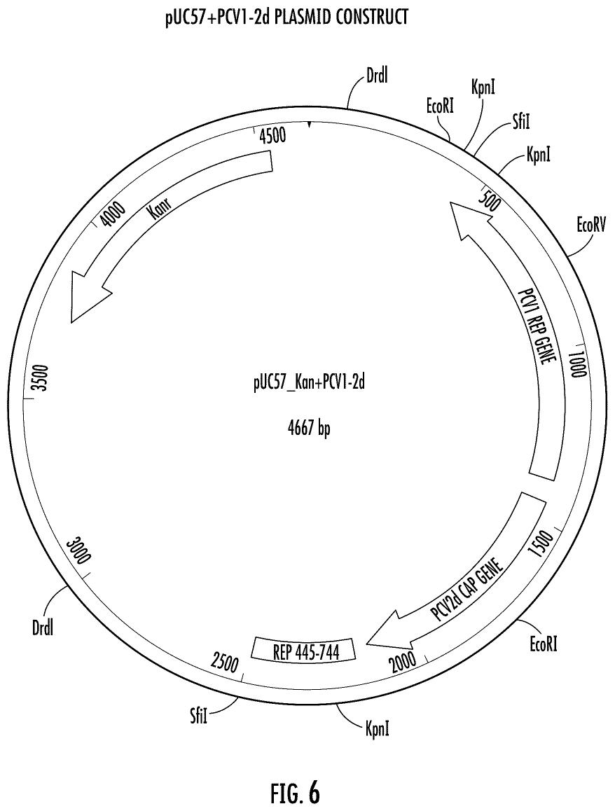

GenScript USA utilized the pUC57-Kan plasmid for cloning, the sequence of which corresponds to SEQ ID NO: 20. The sequence of the PCV2d capsid gene corresponding to SEQ ID NO: 18 was derived from a Zoetis isolate originally referred to as ‘PCV2b-Divergent’, which was disclosed in WO2015/048115 A1 (Zoetis LLC). The genomic sequence for this PCV2d isolate, which was subsequently renamed ‘PCV2d, Isolate Z12’, corresponds to SEQ ID NO: 21. Genscript USA named the pUC57 plus PCV1-2d DNA plasmid construct (depicted in FIG. 6) ‘DIV Clone_pUC57-Kan’.

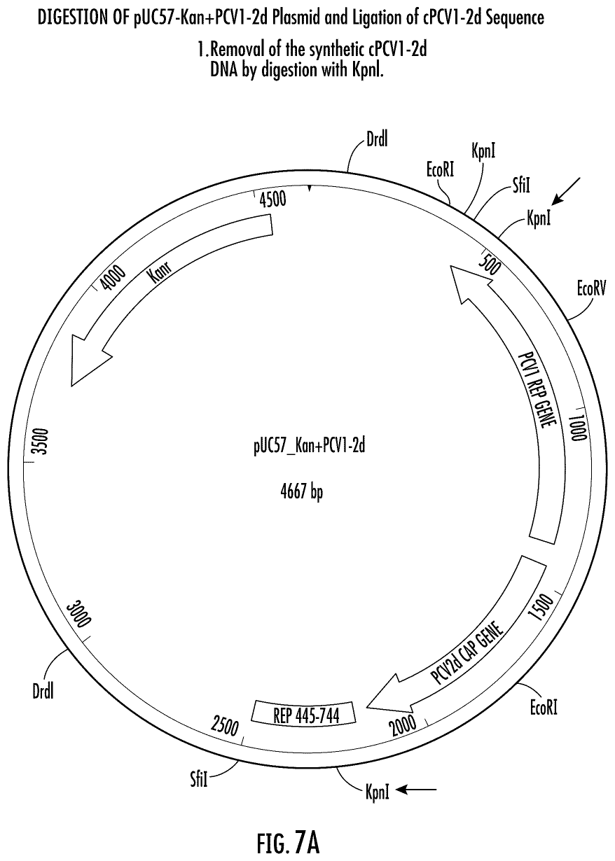

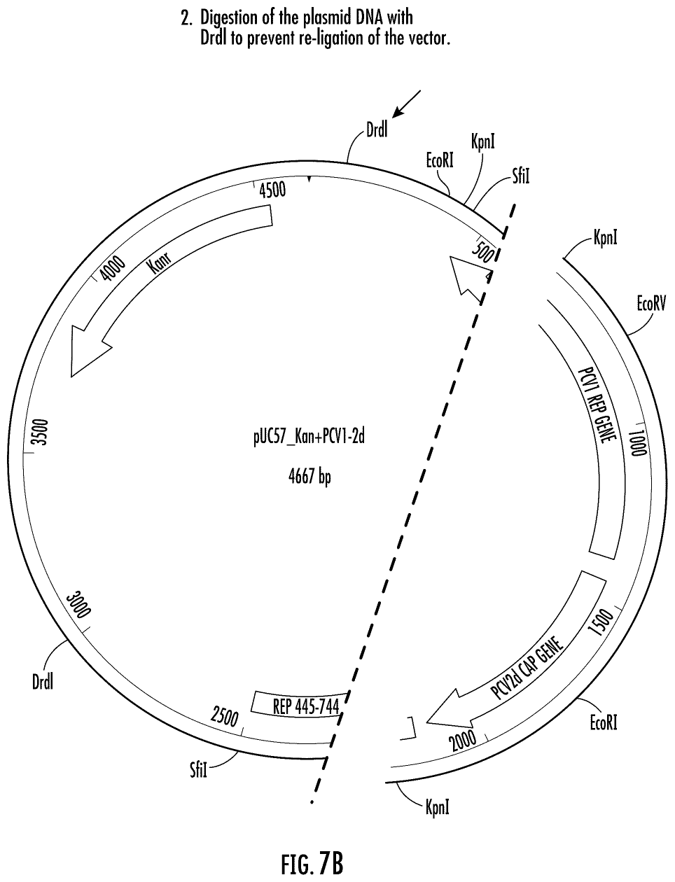

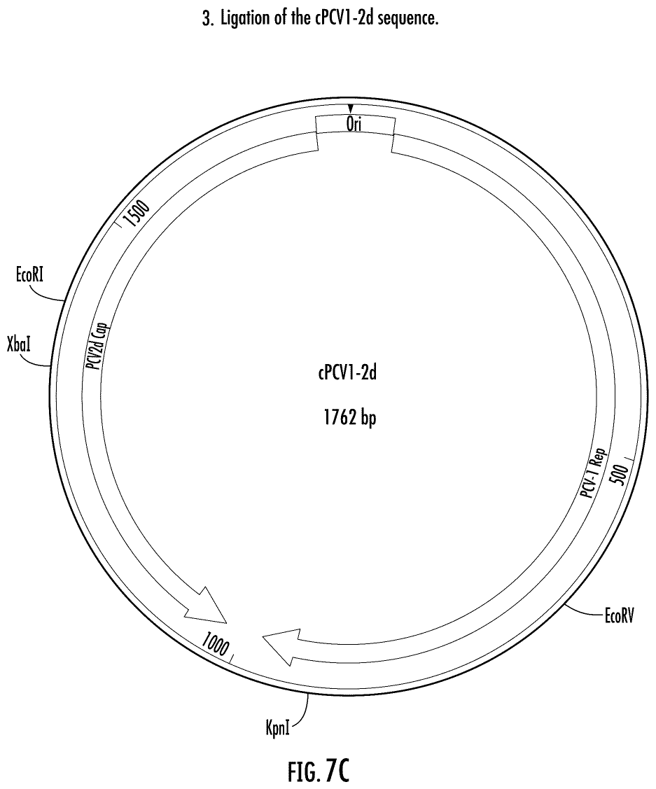

The pUC57+PCV1-2d plasmid construct (GenScript DIV Clone_pUC57-Kan) was digested with Kpnl restriction enzyme to release the PCV sequences (PCV1-2d region, 1762 bp, SEQ ID NO: 17). The digested material was then precipitated with sodium acetate and ethanol to inactivate Kpnl enzyme and concentrate the digested DNA. A second restriction enzyme digest with Drdl was carried out to further digest the plasmid sequences that are devoid of the PCV sequence. This second digestion was carried out to aid in preventing full construct reassembly during the PCV1-2d DNA ligation step. After an additional precipitation with sodium acetate and ethanol, the digested DNA was ligated with ElectroLigase for 30 minutes at room temperature. The ElectoLigase was inactivated with a heating step at 65° C. for 20 minutes and the ligated DNA was transfected into SK-B7 cells using Lipofectamine 3000 reagent. The process of digestion of the pUC57-Kan+PCV1-2d plasmid and ligation of the cPCV1-2d sequence is schematically depicted collectively in FIGS. 7A, 7B, and 7C. Four days post-transfection, the supernatant and cells were passaged into a T-75 flask. The PCV1-2d virus was then passaged and expanded into a T-160 flask for 2 passages and then into 850 cm2 roller bottles. A total of 9 harvests of P5 virus from 6 roller bottles was pooled to make the Pre-Master Seed Virus.

### Virus Construction

### Materials

The following materials were used during plasmid digestion, precipitation, and ligation of the pUC57+PCV1-2d plasmid construct to free the cPCV1-2d region.

TABLE 5
Material List of Vendor Sourced Components for
Plasmid Digestion, Precipitation and Ligation

| Vendor | Catalog Number | Material Description |
|---|---|---|
| Sigma-Aldrich | W4502-1L | Water-Molecular Biology |
| | | Grade |
| New England BioLabs | B7201S | 10X NEB Buffer 1.1 |
| New England BioLabs | R0142L | Kpnl Restriction Enzyme |
| Sigma | S-7899 | 3M NaAcetate |
| Pharmco-AAPER | 111000200 | 200 proof ethyl alcohol |
| New England BioLabs | B7204S | Cut Smart Buffer |
| New England BioLabs | R0530L | Drdl Restriction Enzyme |
| New England BioLabs | M0369S | ElectroLigase |
| New England BioLabs | B0369S | ElectroLigase Buffer |

Once the cPCV1-2d region was freed from the plasmid construct, it was transfected into SK-B7 cells propagated in Dulbecco's Modified Eagle Medium (DMEM) media without lactalbumin hydrolysate (LAH) containing 5% Fetal Bovine Serum (FBS), 1× GlutaMAX and Gentamicin at 20 μg/mL using Lipofectamine 3000 reagent. Tryple Select was used as the disassociation reagent for expansion passages.

TABLE 6
Material List of Vendor Sourced Components
for Transfection and Expansion

| | Catalog | Material |
|---|---|---|
| Vendor | Number | Description |
| Invitrogen | 100022050 | Lipofectamine 3000 |
| Gibco | 31985-062 | OptiMem |
| Gibco | 35050-061 | GlutaMAX |
| Gibco | 15710-066 | Gentamicin |
| Gibco | 12563-011 | TrypLE Select |
| Gibco | 14190-144 | DPBS |

TABLE 7
Material List of Internally Sourced
Components for Transfection and Expansion
Material Description

DMEM w/o LAH
Fetal Bovine Serum

Transfection of cPCV1-2d DNA

SK-B7 cells (Zoetis' proprietary cell line) were prepared by planting at 7.5×105 cells per well into 6-well plates in DMEM media without LAH containing 2% FBS, 1× GlutaMAX and Gentamicin at 20 μg/mL. The following day, the media was replaced with fresh media containing 2% FBS per well prior to transfection and incubated for 1 hour at 37° C. 492 ng/uL/well of digested and re-ligated PCV1-2d chimeric DNA was combined with 10 μL of Lipofectamine™ 3000 reagent (Invitrogen) in 250 μL of OptiMEM media. 250 μL of this mixture was transfected into each well of the culture of SK-B7 cells. Cells were incubated at 37° C. in a humidified incubator with 5% CO2 for four days before Passage 1 and expansion to a 75 cm2 flask.

Passage and Expansion of the cPCV1-2d Virus

All passages used DMEM media without LAH containing 2% FBS, 1× GlutaMAX and Gentamicin at 4 mL/2L as the cell culture media and DPBS as a rinse media prior to cell disassociation with TrypLET Select Enzyme (Gibco).
Passage 1; After the transfected SK-B7 cells were incubated for 4 days, the cells were expanded to a 75 cm2 flask.
Passage 2; 4 days later: Expanded to a 160 cm2 flask.
Passage 3; 4 days later: Passaged into a fresh 160 cm2 flask.
Harvest; 8 days later: Cell culture fluid was harvested by centrifugation to remove cellular debris. Supernatant was collected and labeled as ‘PCV1-2d P3’.

### Pre-Master Seed Virus Preparation

The following materials were used during the cPCV1-2d pre-MSV preparation on SK-B7 cells.

TABLE 8
Material List of Vendor Sourced Components
for Pre-MSV Preparation

| | Catalog | Material |
|---|---|---|
| Vendor | Number | Description |
| Gibco | 35050-061 | GlutaMAX |
| Gibco | 15710-066 | Gentamicin |
| Gibco | 12563-011 | TrypLE Select |
| Gibco | 14190-144 | DPBS |

TABLE 9
Material List of Internally Sourced
Components for Pre-MSV Preparation
Material Description

DMEM w/o LAH
Fetal Bovine Serum

### Methods

Passage 4; 31 days after Passage 3, new SK-B7 cells (P33) were planted in a 160 cm2 flask containing DMEM media without LAH containing 2% FBS, 1× GlutaMAX and Gentamicin at 20 μg/mL and were infected with virus labelled ‘PCV1-2d P3’ at an MOI of 0.01 and incubated at 37° C. in a humidified incubator with 5% CO₂ for three days.

Passage 5; 3 days after Passage 4: Infected cells and a portion of cell culture fluids were expanded by transferring cells and a 1:4 dilution of spent fluids to new 490 cm2 roller bottle with fresh media added to complete the dilution. Infected cells were placed back into the incubator and incubated at 37° C. in a humidified incubator with 5% CO₂ for 3 days before Harvest 1.

Harvest 1; 3 days after Passage 5: 80% of the cell supernatant was removed and replaced with fresh media (FBS concentration of 0.5%).

Harvest 2; 1 day later: 80% of the cell supernatant was removed and replaced with fresh media (FBS concentration of 0.5%).

Harvest 3; 3 days later: 80% of the cell supernatant was removed and replaced with fresh media (FBS concentration of 0.5%).

Harvest 4; 2 days later: 80% of the cell supernatant was removed and replaced with fresh media (FBS concentration of 0.5%).

Harvest 5; 2 days later: 80% of the cell supernatant was removed and replaced with fresh media (FBS concentration of 0.5%).

Harvest 6; 3 days later: 80% of the cell supernatant was removed and replaced with fresh media (FBS concentration of 0.5%).

Harvest 7; 2 days later: 80% of the cell supernatant was removed and replaced with fresh media (FBS concentration of 0.5%).

Harvest 8; 2 days later: Roller bottle was placed in a −80° C. freezer.

Pre-master seed virus was prepared from pooled bulk harvests 1 through 8 and dispensed into appropriately sized aliquots, labeled and frozen at −80° C.

TABLE 10
Pre-Master Seed Virus Passage History

| Day of | | |
|---|---|---|
| Passage | | Passage |
| Initiation | Activity | # |
| Day 45 | Inoculated SK-B7 (P33) cells with ‘PCV1-2d P3’ | 4 |
| | virus into a 160 cm² flask | |
| Day 48 | Expanded infected cells into a 490 cm² roller | 5 |
| | bottle with 1:4 split of spent culture media and | |
| | cells with fresh media | |
| Day 51- | Bulk harvest/refeed of 80% volume (total of 7 | 5 |
| Day 64 | times) | |
| Day 66 | Final bulk harvest (#8) was taken by freezing the | 5 |
| | entire contents of the roller bottle at −80° C. | |
| | Material was thawed and pooled with harvests | |
| | 1-7; aliquot as pre-MSV and labelled cPCV1-2d, | |
| | x + 5 pre-MSV | |

### Pre-Master Seed Virus Testing Summary

The cPCV1-2d, x+5 pre-Master Seed Virus (MSV) was tested by Zoetis VMRD, Kalamazoo, Michigan, USA using the BacT Alert Sterility test method and tested for the absence of mycoplasma by PCR. No bacterial or fungal contamination was found with the BacT Alert Sterility test and the test for mycoplasma indicated the absence of mycoplasma (results not shown).

cPCV1-2d Production Methods
Infection of SK-B7 cells with cPCV1-2d

Swine Kidney (SK)-B7 cells are grown in Pfizer Minimum Essential Medium (PMEM), 30 μg/mL gentamicin sulfate, 1% L-glutamine, and 5% fetal bovine serum. The resulting cells are transferred to microcarriers (Cytodex or Solohill) and subsequently infected with cPCV1-2d virus using OptiMEM Media, up to 0.5% lactabumin hydrosolate (LAH), up to 1% L-glutamine, and up to 2% fetal bovine serum upon initial infection, but up to 0.5% fetal bovine serum upon harvests/re-feeds. All harvests are pooled, the pool is filtered through a 10 micron filter, and the resulting lysates are used to prepare a pre-master seed and subsequent master seed.

The medium which is used for producing virus seeds is the same as that used in producing virus stock. For the growth medium, PMEM, OptiMEM, or equivalent is the basal medium which can be used for planting the SKB7 cell line for outgrowth. The growth medium can be supplemented with up to 10% bovine serum, up to 0.5% lactalbumin hydrolysate, up to 0.5% bovine serum albumin, and up to 30 μg/mL gentamicin. For the virus propagation medium, PMEM, Optimem, or equivalent is used. The virus propagation medium can be supplemented with up to 0.5% lactalbumin hydrolysate, up to 2% bovine serum, up to 0.5% bovine serum albumin, and up to 30 μg/mL gentamicin. Up to 5 g/L glucose and up to 5 mmol/L L-glutamine can be added to the growth medium and/or the virus propagation medium as required to sustain the cells.

Seed virus is diluted in growth basal medium to provide a multiplicity of infection (MOI) of 0.01 to 0.08.

Cultures of SK-B7 cells are initially co-inoculated with working seed virus at the time of cell planting. The cPCV1-2d infected cells are incubated for two (2) to 6 days (target 3 days) at 36±2° C. when the harvest fluids containing microcarriers are allowed to settle for 30 minutes, 70% of the resulting supernatant is removed and replaced with fresh media; this process is repeated up to a total of 6 times, at which all the fluids are removed at the final harvest. Total time for growth through all harvests is 36 days (target 18 days). The cPCV1-2d virus causes observable cytopathic changes during viral replication. At harvest, rounding of cells and considerable floating debris is observed. Cultures are also observed for visual evidence of bacterial or fungal contamination. The incubation time between harvests for the cPCV1-2d antigen is provided in Table 11 below:

TABLE 11
Minimum and Maximum Times for Harvesting cPCV1-2d Antigen

| | Minimum / | Temperature |
|---|---|---|
| Method | Maximum Time | Range |
| Multi-harvest/Re-feed | 2 to 6 days (each | 36 ± 2° C. |
| | harvest); up to 36 | |
| | total days | |

The cPCV1-2d culture fluids are harvested into sterile vessels and are sampled for mycoplasma contamination using known methods. Multiple harvests may be conducted from roller bottles, bioreactors and perfusion vessels.

In some embodiments, the harvested cPCV1-2d virus fluids are clarified through filters resulting in a final filter pore size of 10 microns and then concentrated. For example, one or more antigen lots may be concentrated (e.g., up to 60×) by ultrafiltration. The concentrates may be washed via diafiltration with balanced salt solution to reduce serum proteins.

In some embodiments, the concentrated and washed fluids are characterized via live replicating viral titer, and formulated to the target dose with an appropriate extender and stabilizer (e.g., for example stabilizer containing dextran, lactose, sorbitol, casein and KR Hals extender without phenol red and lactalbumin hydrosolate), pH adjusted, and lyophilized. Product is freeze dried in suitable lyophilization equipment. The process may begin by freezing the shelves to a temperature of −40° C. or colder and holding at this temperature for a minimum of 3 hours before initiating the vacuum drying cycle. The drying cycle should include a primary and secondary drying time. The primary maximum temperature is 10° C. for a specified length of time which is a function of fill volume and container size until primary drying is complete. Secondary drying continues with the shelf temperature maintained at 32° C. (minimum 27° C. to maximum 37° C.) for a range of 2 to 16 hours (target of 8 hours). Upon completion of secondary drying, the product is vacuum stoppered or backfilled with dry nitrogen and stoppered.

In some embodiments, the cPCV1-2d virus can be subjected to a method of inactivation. In further embodiments, one or more antigen lots may be concentrated prior to the inactivation. The method of inactivation, attenuation, or detoxification of the cPCV1-2 virus will now be described. After cPCV1-2d antigen concentration, B-propiolactone (BPL) is added to the pooled cPCV1-2d viral material to obtain an approximate concentration of 0.2% v/v. The pooled viral fluids are then agitated for a minimum of 15 minutes and then the inactivating bulk antigen fluids are transferred to a second sterile vessel. The transferred antigen fluids are maintained at 2-7° C., with constant agitation, for a minimum of 24 hours. After a minimum of 24 hours, a second addition of 0.2% v/v of BPL is added to the pooled suspension. The contents are subsequently agitated, transferred to a third vessel, and maintained at 2-7° C., with constant agitation, for an additional time of not less than 84 hours. In general, the total inactivation time is not less than 108 hours and not more than 120 hours. One embodiment of a suitable inactivation method is summarized in Table 12 below.

TABLE 12
Inactivation Method

| | | | Time-Hours |
|---|---|---|---|
| Inactivant | Final Concentration | Temp. Range | (Min/Max) |
| β-propiolactone | 0.4% v/v(2 × 0.2% | 2-7° C. | 108-120 |
| (BPL) | v/v additions) | (w/Agitation) | |

The inactivation is terminated by the addition of a final concentration of not more than 0.1 M solution of sodium thiosulfate. The pH of the inactivated antigen stock is adjusted to about 6.8 using NaOH or HCl. Following inactivation, a representative sample is taken from the pool and tested for completion of inactivation. The inactivated cPCV1-2d antigen product is standardized to a meet a target antigen concentration as measured via potency ELISA. In one embodiment, the final composition is prepared by combining the inactivated cPCV1-2d virus with a suitable adjuvant and/or other pharmaceutically acceptable carrier. In some embodiments, at least one additional swine antigen may be added to the composition.

### Example 3 Dose Titration of Porcine Circovirus Type 1-Type 2d Chimera, Modified Live Virus Administered to Piglets at 3 Days of Age Followed by Challenge with a Virulent PCV2d Isolate

The objective of the study described in this example was to evaluate the efficacy of a Porcine Circovirus Type 1-Type 2d Chimera, Modified Live Virus candidate when administered intramuscularly once to pigs at approximately 3 days of age at different doses. The PCV1-2d modified live chimeric whole virus was grown on the SK-B7 PERV negative cells described in Example 1.

In this study, 12 clinically healthy pregnant sows, PCV2 seronegative and without apparent disease were brought onto a Zoetis Research Farm site ˜3 weeks pre-farrow. After farrowing, piglets originating from the dams were tested and found to be seronegative and PCV2 viremia free. There were 159 piglets originally enrolled in the study across four treatment groups: 40 in T01, 43 in T02, 41 in T03, and 35 in T04. All eligible piglets were enrolled. On Day 0/1, when animals were 2-9 days of age, T01 were vaccinated in the left neck with 1 mL of Saline as a control group and T02-T04 animals were vaccinated in the left neck with 1 mL of Porcine Circovirus Type 1-Type 2d Chimera, Modified Live Virus at targeted concentrations of 2.5, 3.5, and 4.5 log 10TCID50/mL. The actual doses for T02, T03, and T04 were determined on study day 0 and were 2.1, 3.1, and 4.1 log 10TCID50/mL, respectively. Due to housing constraints, some pigs were randomly culled prior to challenge, leaving 28 animals per treatment group to be challenged on Day 58/59 (eight weeks after vaccination) with PCV2d, given 2 mL intranasally (1 mL/nare) and 2 mL intramuscularly. The challenge strain was Porcine Circovirus-Type 2d, Isolate Z12, the genomic sequence for which is SEQ ID NO: 21. Challenged piglets were necropsied three weeks after challenge on Day 79/80. The study design is shown below in Table 13.

TABLE 13
Study design

| | | | | |
|---|---|---|---|---|
| Target | Actual | | | |
| Dose | Dose | Vaccination | Challenge | Necropsy |

| | | | | | | |
|---|---|---|---|---|---|---|
| Trt | N* | Description | log₁₀ TCID₅₀/mL | Day 0 | Day 56 | Day 77 |

| | | | | | | |
|---|---|---|---|---|---|---|
| T01 | 28 | Control | N/A | N/A | N/A | 2 mL |
| T02 | 28 | cPCV1-2d | 2.5 | 2.1 | 1 mL^{†}, IM | IN^{(1 ml/nare)}, |
| T03 | 28 | | 3.5 | 3.1 | Left Neck | 2 mL |
| T04 | 28 | | 4.5 | 4.1 | | IM^{(right neck)} |

IM = intramuscular
IM = intranasal
*All eligible animals were enrolled; animals were culled prior to challenge leaving 28 per treatment group.
^{†}The target volume of this study was 1 mL, after this study was complete, it was determined to move forward with a 0.5 mL dose volume.

All animals were tested by PCR throughout the vaccination phase and remained PCR negative to PCV2 at all timepoints pre-challenge. Animals 223 (T02) and 290 (T02) were flagged from analysis because they were removed from the study post-challenge but prior to necropsy. The results from samples collected from piglets removed prior to challenge were included in the analysis.

Blood, fecal swabs, and nasal swabs were collected from sows on Days-1, 7, 14, and 17 to evaluate vaccine virus spread from vaccinated piglets back to the sow. Blood, fecal swabs, and nasal swabs were collected from piglets post-vaccination to evaluate vaccine viremia and vaccine shedding. This information was collected to aid in the design of future studies.

Blood, fecal swabs, and nasal swabs were collected post-challenge to evaluate efficacy parameters of PCV2 viremia and PCV2 fecal and nasal shedding, as well as PCV2 serum antibody level monitoring. Blood EDTA samples were collected post challenge to test for lymphopenia. At necropsy, tissues were collected from the piglets for lymphoid depletion, histiocytic replacement, and immunohistochemistry.

The primary efficacy variables were histopathological lesions (lymphoid depletion & histiocytic replacement) and the amount of PCV2 antigen in tissues in test treatment groups compared to control pigs by Immunohistochemistry scores, and the secondary variables were viremia, fecal shedding, and nasal shedding.

### Study Results

### Serum Antibody Response

The PCV2 ELISA detects PCV2 capsid serum antibody response. All treatment groups were seronegative to PCV2 prior to vaccination and showed an increase in PCV2 antibody titers post-challenge, as indicated in Table 14 below.

TABLE 14
PCV2 ELISA (S/P Ratios) By Treatment and Day of Study

| | PCV2 ELISA (S/P Ratios) Geometric Means | | | | | | | |
|---|---|---|---|---|---|---|---|---|
| | Day | Day | Day | Day | Day | Day | Day | Day |
| Trt | −1/0 | 8/9 | 15/16 | 22/23 | 57/58 | 64/65 | 71/72 | 79/80 |
| T01 (Control) | 0.61 | 0.81 | 0.92 | 0.85 | 0.91 | 0.90 | 0.61 | 0.50 |
| T02 (2.1 log₁₀ TCID₅₀/mL) | 0.66 | 0.91 | 1.01 | 0.92 | 0.60 | 0.56 | 0.45 | 0.43 |
| T03 (3.1 log₁₀ TCID₅₀/mL) | 0.65 | 0.79 | 0.85 | 0.99 | 0.65 | 0.65 | 0.52 | 0.59 |
| T04 (4.1 log₁₀ TCID₅₀/mL) | 0.57 | 0.86 | 0.78 | 0.81 | 0.81 | 0.71 | 0.63 | 0.71 |

S/P Ratios: positive if ≤0.5, negative if >0.5.

PCV2 Viremia (ddPCR)

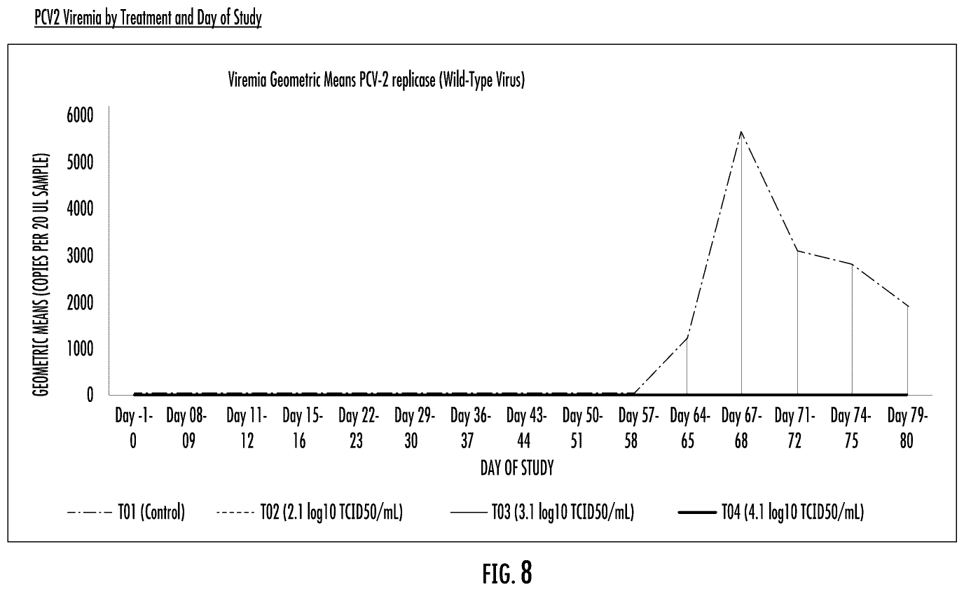

All animals remained PCV2 negative prior to challenge (Day 58/59). T01 control animals had the highest geometric mean of PCV2 viremia (geometric mean 5654.55 copies per 20 μl on Day 67/68) compared to vaccinated animals (T02-T04 geometric means all <1 copy per 20 uL) (Table 15, FIG. 8). T02 and T03 had the lowest number of animals ever PCV2 viremic (Table 16).

TABLE 15
PCV2 Viremia (Copies per 20 μL sample) Detected by ddPCR

| | Geometric Means PCV2 Viremia (Challenge Virus) | | | | | | | | | | | | | | |
|---|---|---|---|---|---|---|---|---|---|---|---|---|---|---|---|
| | Day | Day | Day | Day | Day | Day | Day | Day | Day | Day | Day | Day | Day | Day | Day |
| Treatment | −1/0 | 8/9 | 11/12 | 15/16 | 22/23 | 29/30 | 36/37 | 43/44 | 50/51 | 57/58 | 64/65 | 67/68 | 71/72 | 74/75 | 79/80 |
| T01 (Control) | 0.00 | 0.00 | 0.00 | 0.00 | 0.00 | 0.00 | 0.00 | 0.00 | 0.00 | 0.00 | 1224.17 | 5654.55 | 3075.23 | 2807.36 | 1921.88 |
| T02 (2.1 log10/mL) | 0.00 | 0.00 | 0.00 | 0.00 | 0.00 | 0.00 | 0.00 | 0.00 | 0.00 | 0.00 | 0.00 | 0.00 | 0.18 | 0.00 | 0.00 |
| T03 (3.1 log10/mL) | 0.00 | 0.00 | 0.00 | 0.00 | 0.00 | 0.00 | 0.00 | 0.00 | 0.00 | 0.00 | 0.12 | 0.00 | 0.00 | 0.00 | 0.00 |
| T04 (4.1 log10/mL) | 0.00 | 0.00 | 0.00 | 0.00 | 0.00 | 0.00 | 0.00 | 0.00 | 0.00 | 0.00 | 0.23 | 0.00 | 0.79 | 0.58 | 0.13 |

TABLE 16
PCV2 Viremia Ever Present

| PCV2 Viremia Ever Present | |
|---|---|
| Yes | No |

| Treatment | # | % | # | % |
|---|---|---|---|---|
| T01 (Control) | 28 | 70.0 | 12 | 30.0 |
| T02 (2.1 log₁₀ /mL) | 1 | 2.4 | 40 | 97.6 |
| T03 (3.1 log₁₀ /mL) | 1 | 2.4 | 40 | 97.6 |
| T04 (4.1 log₁₀ /mL) | 3 | 8.6 | 32 | 91.4 |

PCV2 Fecal Shedding (ddPCR)

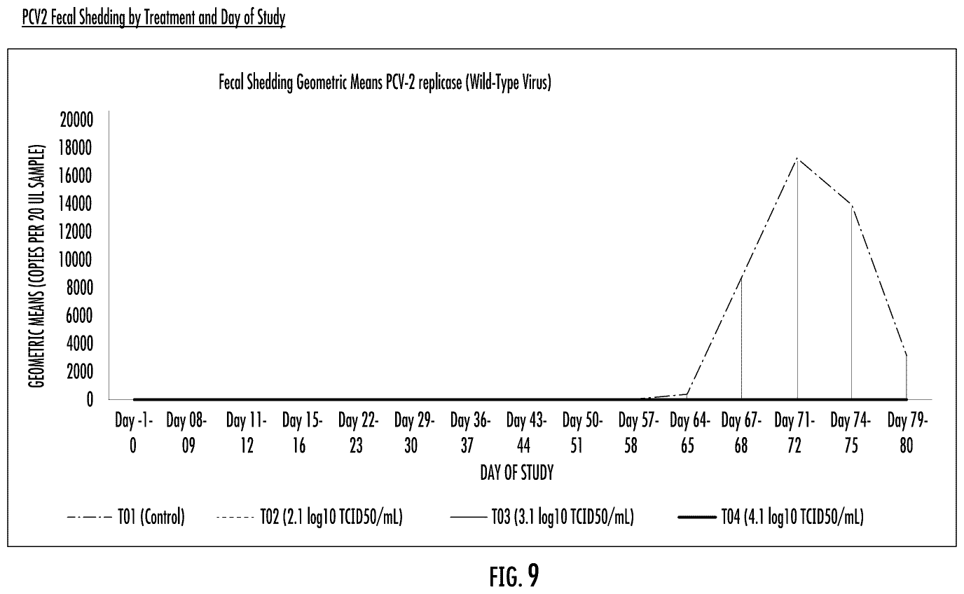

All animals remained PCV2 negative prior to challenge (Day 58/59) in feces. T01 control animals had the highest geometric mean of PCV2 virus in the feces post-challenge (peak geometric mean 17288.69 copies per 20 uL on Day 71/72). T03 shed the lowest geometric mean of virus in feces post-challenge (peak geometric mean 12.06 copies per 20 uL on Day 74/75) (Table 17, FIG. 9). T02 and T03 had the lowest number of animals ever-shedding PCV2 virus in the feces (Table 18).

TABLE 17
PCV2 Fecal Shedding (Copies per 20 μL sample) Detected by ddPCR

| | Geometric Means PCV2 Fecal Shedding (Challenge Virus) | | | | | | | | | | | | | | |
|---|---|---|---|---|---|---|---|---|---|---|---|---|---|---|---|
| | Day | Day | Day | Day | Day | Day | Day | Day | Day | Day | Day | Day | Day | Day | Day |
| Treatment | −1/0 | 8/9 | 11/12 | 15/16 | 22/23 | 29/30 | 36/37 | 43/44 | 50/51 | 57/58 | 64/65 | 67/68 | 71/72 | 74/75 | 79/80 |
| T01 (Control) | 0.00 | 0.00 | 0.00 | 0.00 | 0.00 | 0.00 | 0.00 | 0.00 | 0.00 | 0.00 | 314.55 | 8656.91 | 17288.69 | 14043.01 | 3368.07 |
| T02 (2.1 log10/mL) | 0.00 | 0.00 | 0.00 | 0.00 | 0.00 | 0.00 | 0.00 | 0.00 | 0.00 | 0.00 | 0.18 | 2.23 | 61.29 | 60.48 | 1.63 |
| T03 (3.1 log10/mL) | 0.00 | 0.00 | 0.00 | 0.00 | 0.00 | 0.00 | 0.00 | 0.00 | 0.00 | 0.00 | 0.14 | 1.10 | 11.12 | 12.06 | 0.73 |
| T04 (4.1 log10/mL) | 0.00 | 0.00 | 0.00 | 0.00 | 0.00 | 0.00 | 0.00 | 0.00 | 0.00 | 0.00 | 0.97 | 2.89 | 15.05 | 11.69 | 4.01 |

TABLE 18
PCV2 Fecal Shedding Ever Present

| | Fecal Shedding Ever Present | | | | | | | |
|---|---|---|---|---|---|---|---|---|
| | PCV1 replicase | | | | PCV2 replicase | | | |
| | Yes | | No | | Yes | | No | |
| Treatment | # | % | # | % | # | % | # | % |
| T01 (Control) | 4 | 10.0 | 36 | 90.0 | 28 | 70.0 | 12 | 30.0 |
| T02 (2.1 log₁₀/mL) | 27 | 65.9 | 14 | 34.1 | 16 | 39.0 | 25 | 61.0 |
| T03 (3.1 log₁₀/mL) | 29 | 70.7 | 12 | 29.3 | 16 | 39.0 | 25 | 61.0 |
| T04 (4.1 log₁₀/mL) | 28 | 80.0 | 7 | 20.0 | 17 | 48.6 | 18 | 51.4 |

Nasal Shedding (ddPCR)

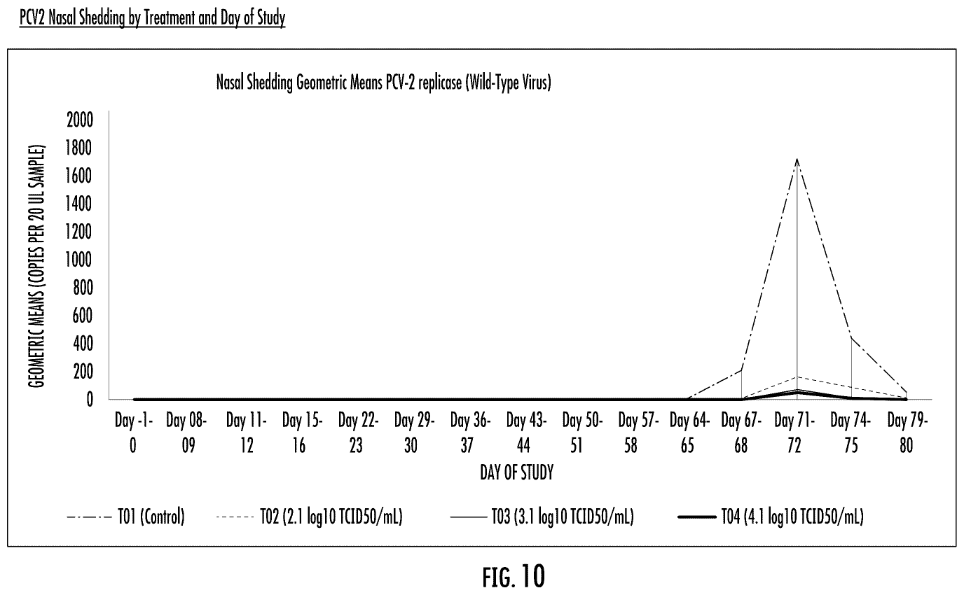

All animals remained PCV2 negative prior to challenge (Day 58/59) in nasal secretions. T01 control animals had the highest geometric mean of PCV2 virus in nasal secretions post-challenge (peak geometric mean 1738.66 copies per 20 uL on Day 71/72). T04 shed the lowest geometric mean of PCV2 virus in nasal secretions post-challenge (peak geometric mean 49.49 copies per 20 uL on Day 71/72) (Table 19, FIG. 10). T02 had the fewest animals ever-nasal shedding (Table 20).

TABLE 19
PCV2 Nasal Shedding (Copies per 20 μL sample) Detected by ddPCR

| | Geometric Means PCV2 Nasal Shedding (Challenge Virus) | | | | | | | | | | | | | | |
|---|---|---|---|---|---|---|---|---|---|---|---|---|---|---|---|
| | Day | Day | Day | Day | Day | Day | Day | Day | Day | Day | Day | Day | Day | Day | Day |
| Treatment | −1/0 | 8/9 | 11/12 | 15/16 | 22/23 | 29/30 | 36/37 | 43/44 | 50/51 | 57/58 | 64/65 | 67/68 | 71/72 | 74/75 | 79/80 |
| T01 (Control) | 0.00 | 0.00 | 0.00 | 0.00 | 0.00 | 0.00 | 0.00 | 0.00 | 0.00 | 0.00 | 6.33 | 211.76 | 1738.66 | 445.36 | 66.12 |
| T02 (2.1 log10/mL) | 0.00 | 0.00 | 0.00 | 0.00 | 0.00 | 0.00 | 0.00 | 0.00 | 0.00 | 0.00 | 2.13 | 6.40 | 164.30 | 84.43 | 21.06 |
| T03 (3.1 log10/mL) | 0.00 | 0.00 | 0.00 | 0.00 | 0.00 | 0.00 | 0.00 | 0.00 | 0.00 | 0.00 | 0.69 | 7.33 | 68.41 | 18.90 | 5.72 |
| T04 (4.1 log10/mL) | 0.00 | 0.00 | 0.00 | 0.00 | 0.00 | 0.00 | 0.00 | 0.00 | 0.00 | 0.00 | 3.96 | 5.16 | 49.49 | 13.07 | 3.29 |

TABLE 20
PCV2 Nasal Shedding Ever Present

| PCV2 Nasal Shedding Ever Present | |
|---|---|
| Yes | No |

| Treatment | # | % | # | % |
|---|---|---|---|---|
| T01 (Control) | 28 | 70.0 | 12 | 30.0 |
| T02 (2.1 log₁₀ /mL) | 18 | 43.9 | 23 | 56.1 |
| T03 (3.1 log₁₀ /mL) | 22 | 53.7 | 19 | 46.3 |
| T04 (4.1 log₁₀ /mL) | 22 | 62.9 | 13 | 37.1 |

### Tissues

Tracheobronchial, Mesenteric, and Inguinal lymph nodes, Spleen, Tonsil, Lung, Heart, Liver, Kidney, lleum, Colon, and Thymus were collected at necropsy for the detection of PCV2 antigen by immunohistochemistry and for appropriate tissues, histopathologic examination for PCVAD pathognomonic lesions (lymphoid depletion and histiocytic replacement) was performed. The results are recorded as:

### Lymphoid Depletion

0, normal; 1, mild lymphoid depletion with loss of overall cellularity; 2, moderate lymphoid depletion; 3, severe lymphoid depletion with loss of lymphoid follicle structure.

### Histiocytic Replacement

0, normal; 1, mild histiocytic-to-granulomatous inflammation; 2, moderate histiocytic-to-granulomatous inflammation; 3, severe histiocytic-to-granulomatous inflammation with replacement of follicles.

### Immunohistochemistry

0, negative; 1, less than 10% of the lymphoid follicles have cells with PCV2 antigen staining; 2, 10-50% of the lymphoid follicles contain cells with PCV2 antigen staining; 3, more than 50% of the lymphoid follicles contain cells with PCV2 antigen staining.

Tissue outcomes for the T01 control group vs. the Treatment groups T02, T03, and T04 are shown below in Tables 21-24.

TABLE 21
Lymphoid Depletion: Number and Percent of Animals Effected

| Lymphoid Depletion | |
|---|---|
| Normal | Abnormal |
| (0) | (1, 2, 3) |

| Treatment | # | % | # | % |
|---|---|---|---|---|
| T01 (Control) | 18 | 64.3 | 10 | 35.7 |
| T02 (2.1 log₁₀ /mL) | 17 | 68.0 | 8 | 32.0 |
| T03 (3.1 log₁₀ /mL) | 21 | 72.4 | 8 | 27.6 |
| T04 (4.1 log₁₀ /mL) | 24 | 85.7 | 4 | 14.3 |

TABLE 22
Histiocytic Replacement: Number and Percent of Animals Effected

| Histiocytic Replacement | |
|---|---|
| Normal | Abnormal |
| (0) | (1, 2, 3) |

| Treatment | # | % | # | % |
|---|---|---|---|---|
| T01 (Control) | 21 | 75.0 | 7 | 25.0 |
| T02 (2.1 log₁₀ /mL) | 18 | 72.0 | 7 | 28.0 |
| T03 (3.1 log₁₀ /mL) | 26 | 89.7 | 3 | 10.3 |
| T04 (4.1 log₁₀ /mL) | 27 | 96.4 | 1 | 3.6 |

TABLE 23
Lymphoid Depletion and/or Histiocytic Replacement:
Number and Percent of Animals Effected

| | Lymphoid Depletion and/or Histiocytic Replacement | | | |
|---|---|---|---|---|
| | Normal | | Abnormal | |
| | (0) | | (1, 2, 3) | |
| Treatment | # | % | # | % |
| T01 (Control) | 16 | 57.1 | 12 | 42.9 |
| T02 (2.1 log₁₀ /mL) | 11 | 44.0 | 14 | 56.0 |
| T03 (3.1 log₁₀ /mL) | 19 | 65.5 | 10 | 34.5 |
| T04 (4.1 log₁₀ /mL) | 23 | 82.1 | 5 | 17.9 |

TABLE 24
Immunohistochemistry: Number and Percent of Animals Effected

| Immunohistochemistry | |
|---|---|
| Normal | Abnormal |
| (0) | (1, 2, 3) |

| Treatment | # | % | # | % |
|---|---|---|---|---|
| T01 (Control) | 7 | 25.0 | 21 | 75.0 |
| T02 (2.1 log₁₀ /mL) | 18 | 72.0 | 7 | 28.0 |
| T03 (3.1 log₁₀ /mL) | 12 | 41.4 | 17 | 58.6 |
| T04 (4.1 log₁₀ /mL) | 18 | 64.3 | 10 | 35.7 |

### CONCLUSIONS

The study was valid in that all the animals met the inclusion criteria and there was no indication of concurrent disease.

All treatment groups (T02-T04) were not biologically different from one another regarding viremia, fecal shedding, and oronasal shedding.

There appears to be a dose-titration effect for all the efficacy parameters evaluated, with T04 (4.1 log 10 TCID50/mL) performing numerically better than T02 (2.1 log 10 TCID50/mL) and T03 (3.1 log 10 TCID50/mL) in nearly all categories.

Overall, tissue outcomes in the T01 control group indicated lower than desired tissue architectural changes. In view of these tissue outcomes, it was decided that alternate animal sources that are more susceptible to PCV2 challenge should be evaluated to allow for better challenge take, and thus a larger difference in tissue outcomes between controls and vaccinates. This was in fact what was observed in the clinical studies described below which employed alternate animal sources as compared to those used in the present example.

### Example 4 Assessment of the Potential Effect of Maternally Derived Antibodies on the Efficacy of Porcine Circovirus Type 1-Type 2d Chimera, MLV Administered to Piglets at 3 Days of Age Followed by Challenge with a Virulent PCV2b Isolate

The objective of the study described in the present example was to evaluate the potential influence of maternally derived antibodies on the efficacy of the Porcine Circovirus Type 1-Type 2d Chimera, Modified Live Virus candidate, administered to three-day-old pigs against a challenge with a virulent strain of PCV2b. The rationale of the present study was based on the EU regulatory guidelines, which require the evaluation of potential maternally derived antibody (MDA) interference of efficacy of vaccines when administered to young animals. The study was designed based on the following guidelines:
Directive 2009/9 EC
European Pharmacopoeia (Ph. Eur.) 8.0, 5.2.7: Evaluation of efficacy of veterinary vaccines of immunosera.
EMA/CVMP/WP/439467/2007: Reflection paper on the demonstration of a possible impact of maternally derived antibodies on vaccine efficacy in young animals

According to EMA/CVMP/WP/439467/2007, the efficacy of the vaccine in animals vaccinated in the presence of MDAs should be, notwithstanding a normal biological variation, similar to that obtained in animals of the same age but vaccinated in the absence of MDAs.

Ph. Eur. Monograph 04/2008:50207 establishes that the efficacy of a product has to be demonstrated for each of the recommended routes and methods of administration and using the recommended schedule to animals of each species and categories for which the use of the product is recommended. In addition, the influence of passively acquired and maternally derived antibodies on the efficacy of a vaccine has to be adequately evaluated.

The test system was a three-day-old pig because this is the age and the target species that the product is intended.

Efficacy in seropositive pigs was evaluated at two target potencies of a 1 mL vaccine or control: 3.2 log 10TCID50/mL (T02 group) and 4.4 log 10TCID50/mL (T03 group). As control non-vaccinated group, one group of seropositive animals was vaccinated with the negative control (saline) product (T01).

The efficacy variables were viremia, fecal and oronasal shedding, serology, presence of PCV2-associated lesions and amount of PCV2 antigen in tissues in test treatment groups T02 and T03 in comparison to T01 negative control pigs.

The test was valid because all pigs from T01 group except one (1120, S/P 0.6141) were seronegative to PCV2 before challenge (Day 96). Furthermore, all animals were PCV2 negative by ddPCR, both in serum and fecal swabs. The oronasal swab of one animal (1141, T02) was positive to ddPCR PCV2 just before challenge (Day 96). Therefore, the data from this animal was flagged out from Day 96 through the end of the study.

Before vaccination, the levels of PCV2 antibody S/P ratio ranged from 1.537 to 3.821, with a geometric mean of 3.102, 3.023, and 2.976 for T01, T02, and T03 groups, respectively (positive if S/P ratio >0.5).

To ensure challenge susceptibility, the animals were challenged when the levels of MDA detected in the T01 (control group) were sufficiently low (geometric mean S/P ratio 0.197; Day 96). MDA did not interfere with challenge take as seen by the high percentage of pigs in the T01 group that became viremic after challenge (100.0%).

### Study Design

The study design is shown below in Table 25 below. Day 0 was the day of vaccination. Six pregnant sows, seropositive to PCV2 before the start of the study, were used to enroll a total of 81 MDA positive piglets (27 for each treatment group). For treatment administration, at day 0, pigs were vaccinated with a single dose of 1 mL of the Investigative Vaccine Product (IVP) or Control Product (CP). Vaccination was done intramuscularly (IM) into the right neck. Pigs were challenged with a total of 3 mL of PCV2b virulent challenge virus, with 2 mL administered intranasally and 1 mL administered intramuscularly in the left neck.

TABLE 25
Study design

| | | | Vaccination | Challenge | End of |
|---|---|---|---|---|---|
| Treatment | N | Test Article | D 0 | D96* | study |
| T01 | 27 | Saline solution (CP) | N/A | PCV2b | 21 days |
| T02 | 27 | cPCV1-2d (3.2 | 1 mL, IM | 2 mL IN (1 | post- |
| | | log₁₀ TCID₅₀/mL) | Right | mL/nostril), | challenge |
| T03 | 27 | cPCV1-2d (4.4 | Neck | 1 mL IM | |
| | | log₁₀ TCID₅₀/mL) | | (left neck) | |

N: number; IM: intramuscular; IN: intranasal
*When the levels of MDAs in the T01 group measured by ELISA become low or undetectable

As indicated in the above table, a PCV2b challenge strain was used. Both PCV2b and PCV2d are the predominant PCV2 genotypes found on farms today. Therefore, it was important for the applicant to assess whether the cPCV1-2d modified live vaccine candidate (which contained the ORF2 from a PCV2d genotype) was capable of cross-protecting against PCV2b. The PCV-Type 2b strain employed was Isolate FD07, the complete genome sequence of which was previously disclosed in US 2009/0162398A1. The dose of challenge strain administered to each animal was 6.42+0.5 TCID50 per animal.

### Validity Requirements

The test was valid since all pigs from T01 group except one (1120, S/P 0.6141) were seronegative to PCV2 before challenge (Day 96). Furthermore, all animals were negative to ddPCR PCV2 either in serum or fecal swab. The oronasal swab of one animal (1141, T02) was positive to ddPCR PCV2 just before challenge (Day 96). Therefore, the data from this animal was flagged out from Day 96 until the end of the study.

### Viremia

PCV1 Viremia (Vaccine Virus)
All pigs were PCV1 ddPCR negative in serum before vaccination (Day 0).

### PCV2 Viremia (Challenge Virus)

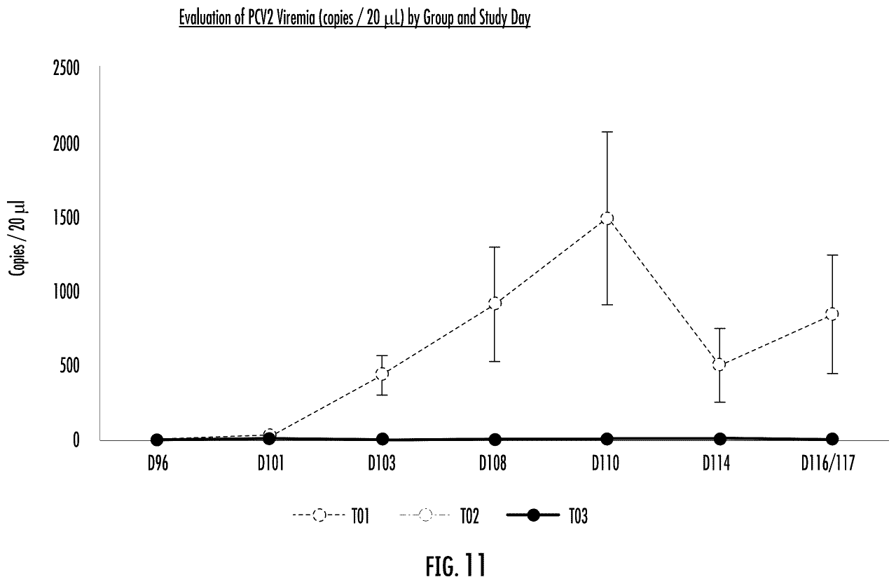

Table 26 and FIG. 11 summarize the viremia results. Back-transformed Least squares means (LSM) and pairwise treatment comparisons at each time point between groups are shown in Table 27. All pigs were PCV2 ddPCR negative in serum before vaccination (Day 0) and before challenge (Day 96).

After challenge, 100.0% (22/22) of pigs from T01 group (control group) became viremic. In vaccinated groups, the percentage of pigs ever viremic was 60.0% (12/20) in T02, and 79.2% (19/24) in T03. Significant differences were observed in the percentage of pigs ever viremic between T01 and T02 groups (P=0.0011). No significant differences were observed between T01 and T03 groups (P=0.0502), or between the two vaccinated groups (T02 and T03, P=0.1990).

The amount of viral load detected in serum was significantly (P≤0.05) higher in the T01 group compared to T02 from Day 101 (5 days post-challenge) until the end of the study (21 days post-challenge). Viremia was significantly (P≤0.05) higher in the control animals (T01) compared to T03 from Day 103 (7 days post-challenge) until the end of the study (21 days post challenge). Viremia was significantly (P≤0.05) higher in T03 animals than T02 at Day 101 (5 days post-challenge).

TABLE 26
Summary of post-challenge viremia results by
group and day of study (copies/20 μL serum)

| | | | BT Least | | | | |
|---|---|---|---|---|---|---|---|
| | | | square | | | Lower | Upper |
| Treatment | Study day | N | Means | SE | Range | 95% CB | 95% CB |
| T01 | D 96 | 20 | 0.0 | 0.0 | 0 to 0.6 | 0.0 | 0.1 |
| | D 101 | 21 | 28.1 | 12.4 | 0.6 to 466 | 11.6 | 66.3 |
| | D 103 | 22 | 434.6 | 133.1 | 6.3 to 99700 | 238.0 | 793.2 |
| | D 108 | 20 | 907.4 | 381.5 | 0 to 120900 | 396.8 | 2073.0 |
| | D 110 | 21 | 1487.7 | 583.2 | 18.1 to 47050 | 688.3 | 3214.2 |
| | D 114 | 21 | 503.4 | 245.2 | 1.9 to 30340 | 192.9 | 13010.4 |
| | D 116/117 | 22 | 844.1 | 399.9 | 8.5 to 197500 | 332.4 | 2141.5 |
| T02 | D 96 | 19 | 0.0 | 0.0 | 0 to 0.6 | 0.0 | 0.1 |
| | D 101 | 20 | 2.0 | 1.3 | 0 to 167 | 0.3 | 6.1 |
| | D 103 | 19 | 1.9 | 0.9 | 0 to 42 | 0.5 | 4.5 |
| | D 108 | 19 | 3.6 | 2.0 | 0 to 448 | 1.0 | 9.8 |
| | D 110 | 20 | 4.1 | 2.0 | 0 to 1211 | 1.3 | 10.2 |
| | D 114 | 18 | 8.8 | 5.1 | 0 to 473 | 2.5 | 26.3 |
| | D 116/117 | 20 | 2.1 | 1.5 | 0 to 409 | 0.2 | 7.3 |
| T03 | D 96 | 23 | 0.0 | 0.0 | 0 to 0.6 | 0.0 | 0.1 |
| | D 101 | 21 | 9.4 | 4.4 | 0 to 50490 | 3.5 | 23.0 |
| | D 103 | 24 | 2.0 | 0.9 | 0 to 41 | 0.7 | 4.4 |
| | D 108 | 22 | 2.3 | 1.3 | 0 to 92 | 0.5 | 6.3 |
| | D 110 | 24 | 2.9 | 1.4 | 0 to 106 | 0.9 | 7.0 |
| | D 114 | 23 | 7.3 | 3.9 | 0 to 849 | 2.3 | 19.8 |
| | D 116/117 | 24 | 1.9 | 1.3 | 0 to 478 | 0.2 | 6.0 |

T01: Control animals;
T02: 3.2 Log₁₀ TCID₅₀/ml IVP;
T03: 4.4 Log₁₀ TCID₅₀/mL IVP
N: number;
SE: standard error;
BT: back-transformed

TABLE 27
Summary table of BT Least Squares Means and pairwise treatment comparisons
at each time point for PCV2 viremia results (copies/20 μL of serum)

| | Back-transformed Least Squares Means at different study dates | | | | | | |
|---|---|---|---|---|---|---|---|
| Treatment | D 96 | D 101 | D 103 | D 108 | D 110 | D 114 | D 116/117 |
| T01 | 0.0 | 28.1 | 434.6 | 907.4 | 1487.7 | 503.2 | 844.1 |
| T02 | 0.0 | 2.0 | 1.9 | 3.6 | 4.1 | 8.8 | 2.1 |
| T03 | 0.0 | 9.4 | 2.0 | 2.3 | 2.9 | 7.3 | 1.9 |
| T01 vs T02 | ns (0.5692) | * (0.0002) | * (<0.0001) | * (<0.0001) | * (<0.0001) | * (<0.0001) | * (<0.0001) |
| (P value) | | | | | | | |
| T01 vs T03 | ns (0.6988) | ns (0.0895) | * (<0.0001) | * (<0.0001) | * (<0.0001) | * (<0.0001) | * (<0.0001) |
| (P value) | | | | | | | |
| T02 vs T03 | ns (0.8366) | * (0.0411) | ns (0.9135) | ns (0.5840) | ns (0.6185) | ns (0.8223) | ns (0.9060) |
| (P value) | | | | | | | |

ddPCR positive: >11 copies/20 μL;
ns: not significant (P > 0.05);
* significant (P ≤ 0.05)

### Fecal Shedding

### PCV1 Fecal Shedding (Vaccine Virus):

All pigs were PCV1 ddPCR negative in fecal swabs before challenge (Day 96).

### PCV2 Fecal Shedding (Challenge Virus):

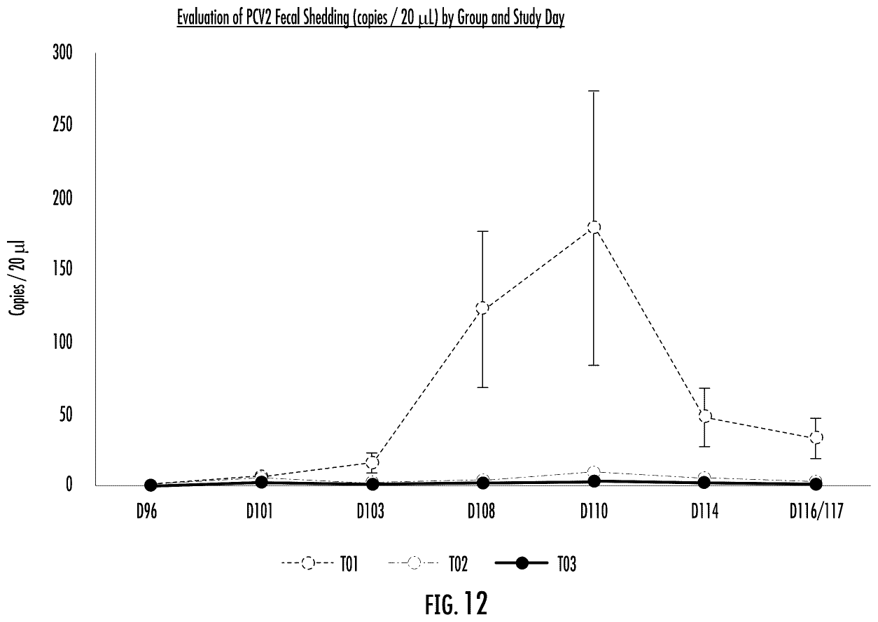

All pigs were PCV2 ddPCR negative in fecal swabs before challenge (Day 96). Table 28 and FIG. 12 summarize the fecal shedding results. Back-transformed Least squares means (LSM) and pairwise treatment comparisons at each time point between groups are shown in Table 29.

After challenge, 72.7% (16/22) of pigs from T01 group (control group) had PCV2 ddPCR positive fecal swabs at least once. In vaccinated groups T02 and T03, the percentage of pigs that ever shed by the fecal route was 30.0% (6/20); and 4.2% (1/24), respectively. Significant differences were observed in the percentage of pigs ever positive between T01 and T02 (P=0.0158); and between T01 and T03 (P=0.0008). No significant differences were observed between the two vaccinated groups T02 and T03 (P=0.0570).

The amount of PCV2 detected in fecal swabs was significantly (P≤0.05) higher in T01 group compared to T02 from Day 103 (7 days post-challenge) until the end of the study (21 days post-challenge). Fecal shedding was significantly higher for T01 animals compared to T03 from Day 101 (5 days post challenge) until the end of the study (21 days post-challenge). Significant differences were observed between the two vaccinated groups (T02 and T03) at day 101 (5-days post-challenge) with T02 animals shedding a higher amount of PCV2 than T03 animals.

TABLE 28
Summary of post-challenge PCV2 fecal shedding results
by group and day of study (copies/20 μL)

| | | | BT Least | | | | |
|---|---|---|---|---|---|---|---|
| | | | squares | | | Lower | Upper |
| Treatment | Study day | N | mean | SE | Range | 95% CB | 95% CB |
| T01 | D 96 | 22 | 0.0 | 0.1 | 0 to 0.6 | 0.0 | 0.2 |
| | D 101 | 22 | 5.4 | 1.4 | 0 to 40 | 3.1 | 8.8 |
| | D 103 | 22 | 16.0 | 4.4 | 0 to 445 | 9.1 | 27.4 |
| | D 108 | 22 | 123.3 | 38.4 | 1.3 to 2298 | 66.8 | 227.0 |
| | D 110 | 21 | 176.2 | 74.6 | 4.6 to 14880 | 76.5 | 404.3 |
| | D 114 | 22 | 48.0 | 14.5 | 0.6 to 1352 | 26.4 | 86.6 |
| | D 116/117 | 22 | 32.9 | 9.5 | 0 to 688 | 18.6 | 57.7 |
| T02 | D 96 | 20 | 0.2 | 0.1 | 0 to 9.4 | 0.0 | 0.3 |
| | D 101 | 20 | 4.0 | 1.1 | 0 to 80 | 2.2 | 6.8 |
| | D 103 | 19 | 1.1 | 0.6 | 0 to 4.9 | 0.2 | 2.6 |
| | D 108 | 20 | 2.7 | 1.2 | 0 to 46 | 1.0 | 6.0 |
| | D 110 | 20 | 8.7 | 4.2 | 0 to 1919 | 3.1 | 21.9 |
| | D 114 | 20 | 4.2 | 1.6 | 0 to 97 | 1.8 | 8.6 |
| | D 116/117 | 20 | 1.4 | 0.7 | 0 to 35 | 0.3 | 3.2 |
| T03 | D 96 | 24 | 0.1 | 0.1 | 0 to 0.6 | 0.0 | 0.2 |
| | D 101 | 24 | 1.6 | 0.6 | 0 to 24.8 | 0.7 | 3.0 |
| | D 103 | 24 | 1.0 | 0.5 | 0 to 7.3 | 0.2 | 2.2 |
| | D 108 | 24 | 1.3 | 0.7 | 0 to 14.9 | 0.3 | 3.2 |
| | D 110 | 23 | 2.4 | 1.4 | 0 to 55 | 0.6 | 6.6 |
| | D 114 | 24 | 1.7 | 0.8 | 0 to 13.7 | 0.6 | 3.8 |
| | D 116/117 | 24 | 0.7 | 0.4 | 0 to 7.8 | 0.0 | 1.8 |

T01: Control animals;
T02: 3.2 Log₁₀ TCID₅₀/ml IVP;
T03: 4.4 Log₁₀ TCID₅₀/mL IVP
N: number;
SE: standard error;
BT: back-transformed

TABLE 29
Summary table of Least Squares Means and pairwise treatment comparisons at each time point for PCV2 shedding results

| | Back-transformed Least Squares Means at different study dates | | | | | | |
|---|---|---|---|---|---|---|---|
| Treatment | D 96 | D 101 | D 103 | D 108 | D 110 | D 114 | D 116/117 |
| T01 | 0.0 | 5.4 | 16.0 | 123.3 | 176.2 | 48.0 | 32.9 |
| T02 | 0.2 | 4.0 | 1.1 | 2.7 | 8.7 | 4.2 | 1.4 |
| T03 | 0.1 | 1.6 | 1.0 | 1.3 | 2.4 | 1.7 | 0.7 |
| T01 vs T02 | ns (0.2214) | ns (0.4397) | * (<0.0001) | * (<0.0001) | * (<0.0001) | * (<0.0001) | * (<0.0001) |
| (P value) | | | | | | | |
| T01 vs T03 | ns (0.5405) | * (0.0038) | * (<0.0001) | * (<0.0001) | * (<0.0001) | * (<0.0001) | * (<0.0001) |
| (P value) | | | | | | | |
| T02 vs T03 | ns (0.5144) | * (0.0408) | ns (0.9197) | ns (0.2960) | ns (0.0805) | ns (0.1201) | ns (0.3693) |
| (P value) | | | | | | | |

ddPCR positive: >45 copies/20 μL;
ns: not significant (P > 0.05);
* significant (P ≤ 0.05)

### Oronasal Shedding

### PCV1 Oronasal Shedding (Vaccine Virus):

All pigs were PCV1 ddPCR negative in oronasal swabs before challenge (Day 96).

### PCV2 Oronasal Shedding (Challenge Virus):

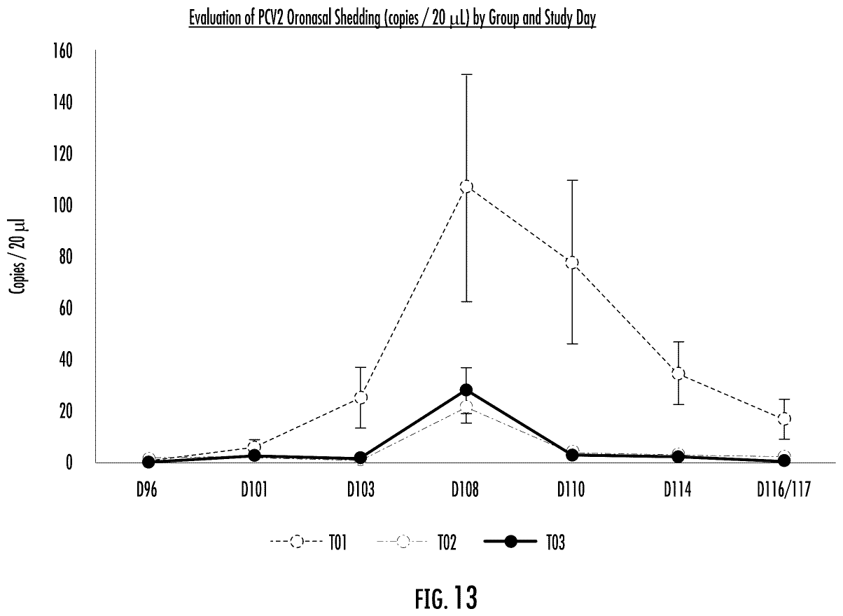

One pig (animal 1141, T02) was removed from the study because it tested PCV2 ddPCR positive in oronasal swabs before challenge (Day 96). All remaining pigs from all treatment groups were PCV2 ddPCR negative in oronasal swabs before challenge (Day 96). Table 30 and FIG. 13 summarize the oronasal shedding results. Back-transformed Least squares means (LSM) and pairwise treatment comparisons at each time point between groups are shown in Table 31.

TABLE 30
Summary of post-challenge PCV2 oronasal shedding
results by group and day of study (copies/20 μL)

| | | | BT Least | | | | |
|---|---|---|---|---|---|---|---|
| | | | squares | | | Lower | Upper |
| Treatment | Study day | N | mean* | SE | Range | 95% CB | 95% CB |
| T01 | D 96 | 22 | 0.0 | 0.0 | 0 to 0.6 | 0.0 | 0.1 |
| | D 101 | 22 | 6.1 | 2.5 | 0 to 1596 | 2.5 | 13.1 |
| | D 103 | 22 | 25.4 | 11.9 | 0 to 8610 | 9.9 | 63.2 |
| | D 108 | 22 | 107.0 | 44.2 | 3.5 to 1906 | 47.3 | 240.6 |
| | D 110 | 21 | 77.7 | 31.6 | 1.8 to 2753 | 34.7 | 172.5 |
| | D 114 | 22 | 34.9 | 12.1 | 2.3 to 712 | 17.5 | 68.6 |
| | D 116/117 | 22 | 17.0 | 7.8 | 0 to 1183 | 6.7 | 41.1 |
| T02 | D 96 | 20 | 0.9 | 0.3 | 0 to 6.1 | 0.5 | 1.5 |
| | D 101 | 20 | 2.5 | 0.5 | 0 to 12.8 | 1.7 | 3.7 |
| | D 103 | 19 | 1.6 | 0.4 | 0 to 8.3 | 0.9 | 2.6 |
| | D 108 | 20 | 19.3 | 5.1 | 0.6 to 101 | 11.4 | 32.2 |
| | D 110 | 20 | 3.9 | 1.3 | 0 to 56 | 2.0 | 7.1 |
| | D 114 | 20 | 2.8 | 0.7 | 0 to 38.8 | 1.6 | 4.5 |
| | D 116/117 | 20 | 2.2 | 0.6 | 0 to 15.6 | 1.1 | 3.7 |
| T03 | D 96 | 24 | 0.3 | 0.1 | 0 to 3.8 | 0.1 | 0.5 |
| | D 101 | 24 | 2.9 | 0.6 | 0 to 19.6 | 1.9 | 4.3 |
| | D 103 | 24 | 1.9 | 0.6 | 0 to 36.6 | 1.0 | 3.2 |
| | D 108 | 24 | 28.1 | 8.9 | 1.2 to 326 | 15.0 | 52.1 |
| | D 110 | 23 | 2.9 | 0.7 | 0 to 59 | 1.7 | 4.6 |
| | D 114 | 24 | 2.1 | 0.4 | 0 to 14.4 | 1.4 | 3.0 |
| | D 116/117 | 24 | 0.6 | 0.1 | 0 to 2.5 | 0.3 | 0.9 |

T01: Control animals;
T02: 3.2 Log₁₀ TCID₅₀/ml IVP;
T03: 4.4 Log₁₀ TCID₅₀/mL IVP
N: number;
SE: standard error;
BT: back-transformed

TABLE 31
Summary table of Least Squares Means and pairwise treatment comparisons
at each time point for PCV2 oronasal shedding results

| | Back-transformed Least Squares Means at different study dates | | | | | | |
|---|---|---|---|---|---|---|---|
| Treatment | D 96 | D 101 | D 103 | D 108 | D 110 | D 114 | D 116/117 |
| T01 | 0.0 | 6.1 | 25.4 | 107.0 | 77.7 | 34.9 | 17.0 |
| T02 | 0.9 | 2.5 | 1.6 | 19.3 | 3.9 | 2.8 | 2.2 |
| T03 | 0.3 | 2.9 | 1.9 | 28.1 | 2.9 | 2.1 | 0.6 |
| T01 vs T02 | * (<0.0001) | ns (0.0673) | * (<0.0001) | * (0.0005) | * (<0.0001) | * (<0.0001) | * (<0.0003) |
| (P value) | | | | | | | |
| T01 vs T03 | * (0.0087) | ns (0.1200) | * (<0.0001) | * (0.0105) | * (<0.0001) | * (<0.0001) | * (<0.0001) |
| (P value) | | | | | | | |
| T02 vs T03 | * (0.0110) | ns (0.6438) | ns (0.7048) | ns (0.3581) | ns (0.4717) | ns (0.3828) | * (0.0013) |
| (P value) | | | | | | | |

ddPCR positive: >11 copies/20 μL;
ns: not significant (P > 0.05);
* significant (P ≤ 0.05)

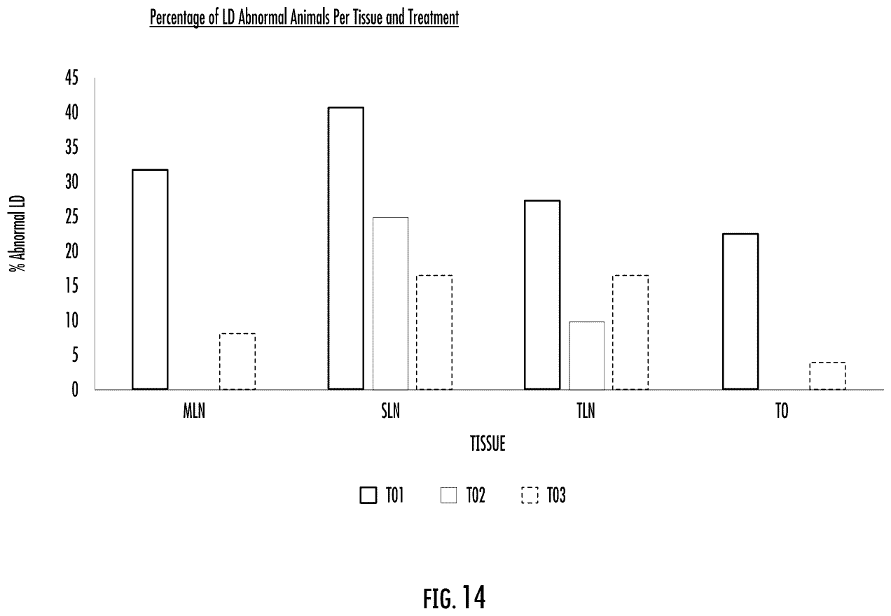

Histopathology and Immunohistochemistry (IHC) and Lymphoid depletion (LD) Table 32 below and FIG. 14 summarize the lymphoid depletion (LD) results per treatment and tissue. Table 33 summarizes the percentage of pigs with abnormal lymphoid depletion (LD) results (scores >0) in any of the four tissue samples evaluated (mesenteric lymph node, superficial inguinal lymph node, tracheobronchial lymph node, and tonsil).

Lymphoid depletion was significantly higher in control animals than in both vaccinated groups, T02 (P=0.0425) and T03 (P=0.0210). No significant differences were observed between T02 and T03 groups (P=0.8360).

TABLE 32
Percentage of animals with LD per tissue and treatment

| | | Lesion score | | | | | |
|---|---|---|---|---|---|---|---|
| | | 0 | | 1 | | 2 | |
| Tissue* | Treatment | Number | % | Number | % | Number | % |
| MLN | T01 | 15 | 68.2 | 6 | 27.3 | 1 | 4.5 |
| | T02 | 20 | 100.0 | 0 | 0 | 0 | 0 |
| | T03 | 22 | 91.7 | 2 | 8.3 | 0 | 0 |
| SLN | T01 | 13 | 59.1 | 6 | 27.3 | 3 | 13.6 |
| | T02 | 15 | 75.0 | 5 | 25.0 | 0 | 0 |
| | T03 | 20 | 83.3 | 3 | 12.5 | 1 | 4.2 |
| TLN | T01 | 16 | 72.7 | 6 | 27.3 | 0 | 0 |
| | T02 | 18 | 90.0 | 2 | 10.0 | 0 | 0 |
| | T03 | 20 | 83.3 | 4 | 16.7 | 0 | 0 |
| TO | T01 | 17 | 77.3 | 4 | 18.2 | 1 | 4.5 |
| | T02 | 20 | 100 | 0 | 0 | 0 | 0 |
| | T03 | 23 | 95.8 | 0 | 0 | 1 | 4.2 |

*MLN: Mesenteric lymph node; SLN: Superficial inguinal lymph node, TLN: tracheobronchial lymph node; TO: tonsil

TABLE 33
Percentage of pigs with abnormal results (score > 0) for
lymphoid depletion (LD) in any of the four tissue samples evaluated

| | Lymphoid |
|---|---|
| Treatment | depletion |
| T01 | 59.1% |
| T02 | 25.0% |
| T03 | 25.0% |
| T01 vs T02 | * |
| (P-value) | 0.0425 |
| T01 vs T03 | * |
| (P-value) | 0.0210 |
| T02 vs T03 | ns |
| (P-value) | 0.8360 |

ns: not significant (P > 0.05);
* significant (P ≤ 0.05)

### Histiocytic Replacement (HR)

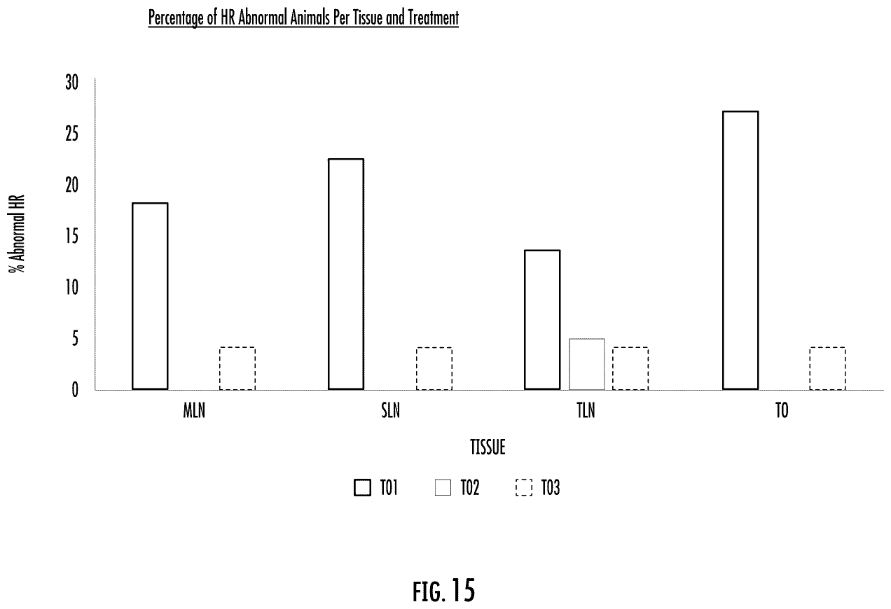

Table 34 and FIG. 15 summarize the histiocytic replacement (HR) results per treatment and tissue. Table 35 summarizes the percentage of pigs with abnormal HR results. No significant differences (P=0.0521) between treatments were observed for histiocytic replacement.

TABLE 34
Percentage of animals with HR per tissue and treatment

| | | Lesion score | | | | | |
|---|---|---|---|---|---|---|---|
| | | 0 | | 1 | | 2 | |
| Tissue* | Treatment | Number | % | Number | % | Number | % |
| MLN | T01 | 18 | 81.8 | 4 | 18.2 | 0 | 0 |
| | T02 | 20 | 100.0 | 0 | 0 | 0 | 0 |
| | T03 | 23 | 95.8 | 1 | 4.2 | 0 | 0 |
| SLN | T01 | 17 | 77.3 | 3 | 13.6 | 2 | 9.1 |
| | T02 | 20 | 100.0 | 0 | 0 | 0 | 0 |
| | T03 | 23 | 95.8 | 0 | 0 | 1 | 4.2 |
| TLN | T01 | 19 | 86.4 | 3 | 13.6 | 0 | 0 |
| | T02 | 19 | 95.0 | 1 | 5.0 | 0 | 0 |
| | T03 | 23 | 95.8 | 1 | 4.2 | 0 | 0 |
| TO | T01 | 16 | 72.7 | 5 | 22.7 | 1 | 4.5 |
| | T02 | 20 | 100.0 | 0 | 0 | 0 | 0 |
| | T03 | 23 | 95.8 | 1 | 4.2 | 0 | 0 |

*MLN: Mesenteric lymph node; SLN: Superficial inguinal lymph node, TLN: tracheobronchial lymph node; TO: tonsil

TABLE 35
Percentage of pigs with abnormal results (score > 0) for histiocytic
replacement (HR) in any of the four tissue samples evaluated

| | Histiocytic |
|---|---|
| Treatment | infiltration |
| T01 | 27.3% |
| T02 | 5.0% |
| T03 | 4.2% |
| T01 vs T02 (P-value) | Not tested |
| T01 vs T03 (P-value) | Not tested |
| T02 vs T03 (P-value) | Not tested |

### LD/HR

Table 36 summarizes the percentage of pigs with abnormal LD/HR results.

When histiocytic infiltration (HI) and lymphoid depletion (LD) were considered together for the analysis, the percentage of pigs with PCV2-associated lesions in any of the lymphoid tissues evaluated was significantly higher in T01 compared to T02 (P=0.0199) and T03 (P =0.0102). No significant differences were observed between the two vaccinated groups (T02 and T03).

TABLE 36
Percentage of pigs with abnormal results (score >
0) for LD/HR in any of the four tissue samples evaluated

| Treatment | LD/HR |
|---|---|
| T01 | 63.6% |
| T02 | 25.0% |
| T03 | 25.0% |
| T01 vs T02 | * |
| (P-value) | 0.0199 |
| T01 vs T03 | * |
| (P-value) | 0.0102 |
| T02 vs T03 | ns |
| (P-value) | 0.8708 |

ns: not significant (P > 0.05);
* significant (P ≤ 0.05)

### Immunohistochemistry (IHC)

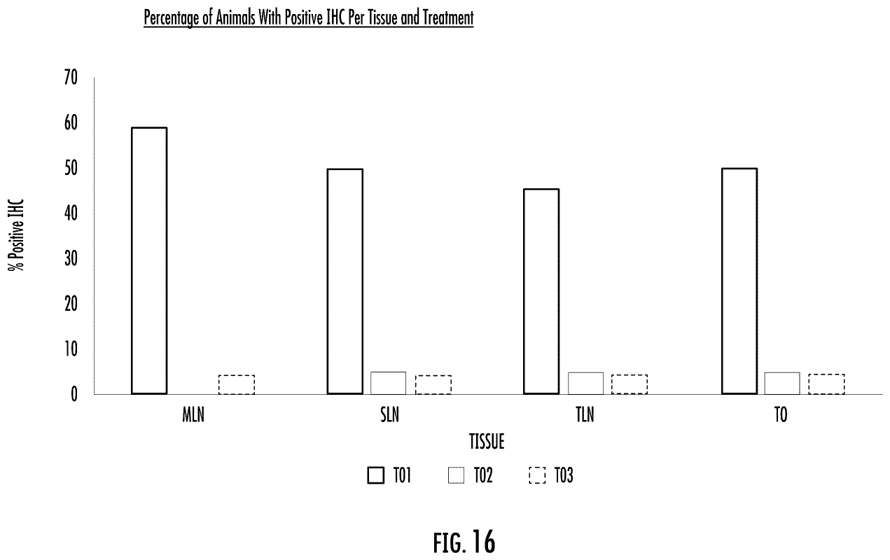

Tissue samples were processed and detected for PCV2 antigen by immunochemistry (IHC). Table 37 and FIG. 16 summarize the IHC results per treatment and tissue. Table 40 summarizes the percentage of pigs with positive IHC results.

The percentage of pigs with positive immunohistochemistry scores in any of the lymphoid tissues evaluated was significantly higher in T01 when compared to T02 (P=0.0019) and T03 (P=0.0010). No significant differences were observed between the two vaccinated groups T02 and T03 (P=0.8776).

TABLE 37
Percentage of animals with IHC per tissue and treatment

| | | Lesion score | | | | | |
|---|---|---|---|---|---|---|---|
| | | 0 | | 1 | | 2 | |
| Tissue* | Treatment | Number | % | Number | % | Number | % |
| MLN | T01 | 9 | 40.9 | 12 | 54.5 | 1 | 4.5 |
| | T02 | 20 | 100.0 | 0 | 0 | 0 | 0 |
| | T03 | 23 | 95.8 | 1 | 4.2 | 0 | 0 |
| SLN | T01 | 11 | 50.0 | 10 | 45.5 | 1 | 4.5 |
| | T02 | 19 | 95.0 | 1 | 5.0 | 0 | 0 |
| | T03 | 23 | 95.8 | 0 | 0 | 1 | 4.2 |
| TLN | T01 | 12 | 54.5 | 9 | 40.9 | 1 | 4.5 |
| | T02 | 19 | 95.0 | 1 | 5.0 | 0 | 0 |
| | T03 | 23 | 95.8 | 1 | 4.2 | 0 | 0 |
| TO | T01 | 11 | 50.0 | 10 | 45.5 | 1 | 4.5 |
| | T02 | 19 | 95.0 | 1 | 5.0 | 0 | 0 |
| | T03 | 23 | 95.8 | 1 | 4.2 | 0 | 0 |

*MLN: Mesenteric lymph node; SLN: Superficial inguinal lymph node, TLN: tracheobronchial lymph node; TO: tonsil

TABLE 38
Percentage of pigs with positive results (score >
0) for IHC in any of the four tissue samples evaluated

| Treatment | IHC |
|---|---|
| T01 | 68.2% |
| T02 | 5.0% |
| T03 | 4.2% |
| T01 vs T02 | * |
| (P-value) | 0.0019 |
| T01 vs T03 | * |
| (P-value) | 0.0010 |
| T02 vs T03 | ns |
| (P-value) | 0.8776 |

ns: not significant (P > 0.05);
* significant (P ≤ 0.05)

### Serology

Serology data before vaccination (Day 0) indicated that all animals met the inclusion criteria for the MDA status before vaccination. All animals had a PCV2 ELISA S/P ratio <0.5. The geometric mean titers were 3.102, 3.023, and 2.976 for T01, T02, and T03 groups, respectively.

The animals were challenged at Day 96, when the MDA from T01 had dropped (S/P ratio <0.5). After challenge (Day 96), PCV2 antibody titers were maintained in all vaccinated groups until Day 101. At Day 103, a boost was observed in both vaccinated groups that was maintained until the end of the study.

The PCV2 antibody titers of groups T02 and T03 were significantly higher (P≤0.05) compared with the control group (T01) from Day 76 until the end of the study. Before that, they were significantly lower from Day 20 to Day 47. No significant differences between groups were observed at Day 60. Significant differences between the two vaccinated groups T02 and T03 were observed at Day 47 and at Day 96.

Table 39 summarizes the serology results. Least square geometric means and significance of treatment comparisons are shown in Table 40.

TABLE 39
Summary of serology results by group and day of study (ELISA S/P ratio)

| | Day of | | Geometric | | | Lower | Upper |
|---|---|---|---|---|---|---|---|
| Treatment | study | N | LSM | SE | Range | 95% CB | 95% CB |
| T01 | D 20 | 24 | 3.719 | 0.222 | 2.668 to 4.745 | 3.304 | 4.175 |
| | D 37 | 24 | 2.270 | 0.153 | 1.217 to 3.541 | 1.982 | 2.585 |
| | D 47 | 24 | 1.898 | 0.136 | 0.919 to 2.658 | 1.643 | 2.178 |
| | D 60 | 23 | 1.967 | 0.139 | 0.432 to 3.629 | 1.706 | 2.253 |
| | D 76 | 23 | 0.926 | 0.090 | 0.276 to 1.819 | 0.756 | 1.112 |
| | D 93 | 22 | 0.534 | 0.072 | 0.156 to 1.111 | 0.399 | 0.682 |
| | D 96 | 22 | 0.197 | 0.056 | 0.002 to 0.848 | 0.092 | 0.313 |
| | D 101 | 22 | 0.189 | 0.05 | 0.001 to 0.761 | 0.084 | 0.303 |
| | D 103 | 22 | 0.533 | 0.072 | 0.094 to 1.545 | 0.398 | 0.681 |
| | D 108 | 22 | 0.646 | 0.077 | 0.011 to 1.108 | 0.501 | 0.805 |
| | D 110 | 22 | 0.732 | 0.081 | 0.392 to 1.322 | 0.580 | 0.899 |
| | D 114 | 22 | 1.383 | 0.112 | 0.72 to 2.072 | 1.173 | 1.613 |
| | D 116/117 | 22 | 1.285 | 0.107 | 0.717 to 2.304 | 1.084 | 1.506 |
| T02 | D 20 | 23 | 1.893 | 0.171 | 0.814 to 3.539 | 1.576 | 2.248 |
| | D 37 | 23 | 1.421 | 0.140 | 0.117 to 2.064 | 1.161 | 1.713 |
| | D 47 | 22 | 1.424 | 0.140 | 0.517 to 2.093 | 1.164 | 1.716 |
| | D 60 | 22 | 1.604 | 0.151 | 0.196 to 2.583 | 1.324 | 1.918 |
| | D 76 | 21 | 1.957 | 0.171 | 1.283 to 2.774 | 1.639 | 2.313 |
| | D 93 | 21 | 2.046 | 0.176 | 1.17 to 2.681 | 1.718 | 2.412 |
| | D 96 | 21 | 0.849 | 0.107 | 0.044 to 1.686 | 0.650 | 1.071 |
| | D 101 | 21 | 1.180 | 0.126 | 0.204 to 1.707 | 0.946 | 1.443 |
| | D 103 | 20 | 2.286 | 0.192 | 1.064 to 3.05 | 1.930 | 2.685 |
| | D 108 | 21 | 2.182 | 0.184 | 0.773 to 2.925 | 1.840 | 2.565 |
| | D 110 | 21 | 1.948 | 0.171 | 0.747 to 2.655 | 1.631 | 2.303 |
| | D 114 | 21 | 2.194 | 0.185 | 0.055 to 3.018 | 1.850 | 2.578 |
| | D 116/117 | 21 | 1.964 | 0.172 | 0.352 to 2.948 | 1.645 | 2.321 |
| T03 | D 20 | 25 | 1.903 | 0.146 | 0.869 to 2.355 | 1.630 | 2.204 |
| | D 37 | 24 | 1.509 | 0.126 | 0.059 to 2.159 | 1.273 | 1.769 |
| | D 47 | 24 | 0.866 | 0.094 | 0.05 to 1.935 | 0.691 | 1.059 |
| | D 60 | 24 | 1.901 | 0.146 | 0.844 to 2.414 | 1.629 | 2.202 |
| | D 76 | 24 | 1.580 | 0.130 | 0.955 to 2.328 | 1.337 | 1.847 |
| | D 93 | 24 | 1.786 | 0.140 | 1.242 to 2.592 | 1.524 | 2.074 |
| | D 96 | 24 | 1.145 | 0.108 | 0.008 to 1.879 | 0.944 | 1.368 |
| | D 101 | 24 | 0.926 | 0.097 | 0.073 to 1.69 | 0.745 | 1.125 |
| | D 103 | 24 | 2.323 | 0.167 | 0.842 to 3.152 | 2.011 | 2.667 |
| | D 108 | 23 | 2.028 | 0.154 | −0.012 to 3.161 | 1.741 | 2.346 |
| | D 110 | 24 | 2.042 | 0.153 | 1.446 to 2.83 | 1.757 | 2.357 |
| | D 114 | 24 | 2.312 | 0.166 | 1.149 to 3.264 | 2.001 | 2.655 |
| | D 116/117 | 24 | 2.111 | 0.156 | 1.217 to 3.176 | 1.819 | 2.433 |

T01: Control animals;
T02: 3.2 Log₁₀ TCID₅₀/ml IVP;
T03: 4.4 Log₁₀ TCID₅₀/mL IVP
N: number;
SE: standard error

TABLE 40
Summary table of Geometric Least Squares Means for serology results

| | Geometric Least Squares Means at different study dates | | | | | | | |
|---|---|---|---|---|---|---|---|---|
| Treatment | D 0* | D 20 | D 37 | D 47 | D 60 | D 76 | D 93 | D 96 |
| T01 | 3.102 | 3.719 | 2.270 | 1.898 | 1.967 | 0.926 | 0.534 | 0.197 |
| T02 | 3.023 | 1.893 | 1.421 | 1.424 | 1.604 | 1.957 | 2.046 | 0.849 |
| T03 | 2.976 | 1.903 | 1.509 | 0.866 | 1.901 | 1.58 | 1.786 | 1.145 |
| T01 vs T02 | Not | * (<0.0001) | * (<0.0001) | * (0.0087) | ns (0.0550) | * (<0.0001) | * (<0.0001) | * (<0.0001) |
| (P-value) | tested | | | | | | | |
| T01 vs T03 | Not | * (<0.0001) | * (<0.0001) | * (<0.0001) | ns (0.7175) | * (<0.0001) | * (<0.0001) | * (<0.0001) |
| (P-value) | tested | | | | | | | |
| T02 vs T03 | Not | ns (0.9603) | ns (0.6131) | * (0.0002) | ns (0.1238) | ns (0.0521) | ns (0.2043) | * (0.0343) |
| (P-value) | tested | | | | | | | |

| | Geometric Least Squares Means at different study dates | | | | | |
|---|---|---|---|---|---|---|
| Treatment | D 101 | D 103 | D 108 | D 110 | D 114 | D 116/117 |
| T01 | 0.189 | 0.533 | 0.646 | 0.732 | 1.383 | 1.285 |
| T02 | 1.180 | 2.286 | 2.182 | 1.948 | 2.194 | 1.964 |
| T03 | 0.926 | 2.323 | 2.028 | 2.042 | 2.312 | 2.111 |
| T01 vs T02 | * (<0.0001) | * (<0.0001) | * (<0.0001) | * (<0.0001) | * (<0.0001) | * (0.0001) |
| (P-value) | | | | | | |
| T01 vs T03 | * (<0.0001) | * (<0.0001) | * (<0.0001) | * (<0.0001) | * (<0.0001) | * (<0.0001) |
| (P-value) | | | | | | |
| T02 vs T03 | ns (0.0771) | ns (0.8746) | ns (0.4843) | ns (0.6546) | ns (0.6060) | ns (0.4906) |
| (P-value) | | | | | | |

Positive >0.5;
ns: not significant (P > 0.05);
* significant (P ≤ 0.05)
*Geometric means

### CONCLUSIONS

Compared to the control group (T01), the vaccinated groups (T02 and T03) demonstrated a statistically significant (P≤0.05) less viral load in blood, fecal and oronasal secretions as well as a percentage of significantly higher levels of PCV2 antibodies after challenge. In addition, both vaccinated groups showed statistically significant (P≤0.05) reduction in the amount of pigs with histopathological lesions associated with PCV2 (lymphoid depletion and lymphoid depletion/histiocytic replacement) as well as a reduction of the amount of pigs with PCV2 antigen in tissues (immunohistochemistry).

Therefore, and under the conditions of the present study, it is concluded that the test vaccine administered to 1-to-3-day old piglets in the face of MDA, induced a significant protection against challenge with a pathogenic strain of PCV2b, thirteen weeks (96 days) post-vaccination, as evidenced by the significant reduction in viremia, fecal, and oronasal shedding as well as a significant increase in PCV2 antibody levels after challenge. Finally, a significant reduction of histopathologic lesions associated with PCV2 as well as significant reduction of PCV2 antigen in tissues in both vaccinated groups was observed.

Significant protection was obtained at both 3.2 and 4.4 Log10 TCID50/mL, with very few significant differences detected between vaccinated groups.

Finally, a significant reduction of histopathological lesions associated with PCV2 as well as significant reduction of PCV2 antigen in tissues in both vaccinated groups was observed.

### Example 5 Evaluation of Porcine Circovirus Type 1-Type 2 Chimera, Modified Live Virus Administered to Piglets at 3 Days of Age with a Needle-free Device or a Needle Followed by Challenge with a Virulent PCV2d Isolate

The objective of the study was to evaluate the efficacy of the candidate Porcine Circovirus Type 1-Type 2d Chimera, Modified Live Virus vaccine when administered intramuscularly with either a needle-free device or a needle and syringe once to pigs at approximately 3 days of age.

Nine sows, seronegative to PCV2 and ddPCR negative to PCV1 and PCV2, were purchased and brought to Zoetis approximately 3 weeks pre-farrow. After farrowing, 115 piglets were enrolled in the study across three treatment groups: 39 in T01 and 38 in each T02 and T03. All eligible piglets were enrolled. On Day 0, when animals were 3-7 days of age, T01 animals were vaccinated in the right neck via a needle-free device with 0.5 mL of saline as a control group and T02-T03 animals were vaccinated in the right neck via either a needle free device (T02) or a needle (T03) with 0.5 mL of Porcine Circovirus Type 1-Type 2d Chimera, Modified Live Virus at 4.1 log 10 TCID50/dose. Piglets were housed separately by treatment group until they were co-mingled prior to challenge.

Piglets were challenged eight weeks post-vaccination via 2 mL intranasally (1 mL per nare) and 1 mL intramuscularly in the left neck with a PCV2d isolate, Isolate Z12 (SEQ ID NO: 21). Piglets were observed daily for clinical signs of PCV2 infection.

Serum, fecal swabs, and oronasal swabs were collected on all piglets once weekly between vaccination and challenge and three times a week post-challenge. Serum was tested via the BioCheck ELISA for PCV2 antibody. Serum, fecal swabs, and oronasal swabs were tested via ddPCR for PCV1 and PCV2 viremia and fecal and nasal shedding. Piglets were necropsied three weeks post-challenge and lymph nodes and tonsil were collected for evaluation of PCV2-related tissue changes and immunohistochemistry.

The study design is shown below in Table 41.

TABLE 41
Study Design

| | Number of | Number of | | Vaccination | Challenge | Necropsy |
|---|---|---|---|---|---|---|
| Trt | Sows | Piglets | Treatment | Day 0 | Day 56 | Day 77 |
| T01 | 3 | 39 | Saline | 0.5 mL NF | 1 mL IM | Necropsy |
| T02 | 3 | 38 | cPCV1-2d MLV | Right Neck | (Left Neck) | and |
| T03 | 3 | 38 | 4.1 TCID₅₀/dose | 0.5 mL N | 2 mL IN | Tissue |
| | | | | Right Neck | (1 mL in | Collection |
| | | | | | each nostril) | |

N = needle
NF = needle-free
IM = intramuscular
IN = intranasal

### Blood Samples and Testing

The blood samples were allowed to clot at room temperature and were processed by PASS lab according to their site standard operating procedures.

Serum was tested at specified time points for PCV2 antibody by the PCV2 BioCheck Elisa (or equivalent).

Serum was also tested for PCV1 and PCV2 viremia by a multiplex ddPCR assay designed with pan-PCV replicase gene primers and probes specific to PCV1 or PCV2 replicase gene.

The assay distinguished between PCV1-2d chimeric vaccine strain and a PCV2 challenge strain based on the PCV1 or PCV2 specific probe. Results are reported in ‘copies/20 uL’.

### Fecal and Nasal Swabs

Swabs collected were tested for PCV1 and PCV2 viremia by a multiplex ddPCR assay designed with pan-PCV replicase gene primers and probes specific to PCV1 or PCV2 replicase gene. The assay distinguished between PCV1-2d chimeric vaccine strain and a PCV2 challenge strain based on the PCV1 or PCV2 specific probe. Results are reported in ‘copies/20 uL’.

### Tissue Scoring

Lymphoid Depletion/Histiocytic Replacement: For appropriate tissues, histopathologic examination for PCVAD pathognomonic lesions (lymphoid depletion and histiocytic replacement). The results are recorded as:

Lymphoid Depletion 0, normal; 1, mild lymphoid depletion with loss of overall cellularity; 2, moderate lymphoid depletion; 3, severe lymphoid depletion with loss of lymphoid follicle structure.

Histiocytic Replacement 0, normal; 1, mild histiocytic-to-granulomatous inflammation; 2, moderate histiocytic-to-granulomatous inflammation; 3, severe histiocytic-to-granulomatous inflammation with replacement of follicles.

Immunohistochemistry: Tissues were also processed for detection of PCV2 antigen by immunohistochemistry (IHC). The results are recorded as 0, negative; 1, less than 10% of the lymphoid follicles have cells with PCV2 antigen staining; 2, 10-50% of the lymphoid follicles contain cells with PCV2 antigen staining; 3, more than 50% of the lymphoid follicles contain cells with PCV2 antigen staining.

### RESULTS

All animals remained ddPCR negative to PCV2 at all timepoints pre-challenge. Animals 553 (T03) and 626 (T01) were flagged from analysis because they were removed from the study post-challenge but prior to necropsy. The results from samples collected from piglets removed prior to challenge were included in the analysis.

### Study Validity

This study is valid in that all piglets were PCV2 viremia free and PCV2 seronegative prior to vaccination.

### Injection Site Reactions

Injection sites were observed and palpated for swelling and redness on Day 1 (Table 42). There were no injection site reactions for any animal on Day 1; therefore, injection sites were not observed on Day 3 according to protocol requirements.

TABLE 42
Injection Site Reactions

Score (Day 1)
0

| Treatment | Number | % |
|---|---|---|
| T01 (Saline NF) | 39 | 100.0 |
| T02 (IVP NF) | 38 | 100.0 |
| T03 (IVP Needle) | 38 | 100.0 |

### Clinical Observations

Animals were observed daily to see if they exhibited PCV2-related clinical signs, which included diarrhea, inappetence, lethargy, pallor/icterus, and respiratory distress. The results are presented in Table 43 below.

TABLE 43
PCV2 Clinical Observations Ever Present by Treatment Group

| | PCV Clinical Observations Ever Present [# of animals (% of animals)] | | | | | | | | | |
|---|---|---|---|---|---|---|---|---|---|---|
| | | | | | | | Pallor/ | | Respiratory | |
| | Diarrhea | | Inappetence | | Lethargy | | Icterus | | Distress | |
| Trmnt | Yes | No | Yes | No | Yes | No | Yes | No | Yes | No |
| T01 (Saline NF) | 6 | 33 | 0 | 39 | 1 | 38 | 0 | 39 | 0 | 39 |
| | (15.4) | (84.6) | (0) | (100) | (2.6) | (97.4) | (0) | (100) | (0) | (100) |
| T02 (IVP NF) | 10 | 27 | 1 | 36 | 1 | 36 | 0 | 37 | 0 | 37 |
| | (27.0) | (73.0) | (2.7) | (97.3) | (2.7) | (97.3) | (0) | (100) | (0) | (100) |
| T03 (IVP Needle) | 7 | 30 | 0 | 37 | 0 | 37 | 0 | 37 | 1 | 36 |
| | (18.9) | (81.1) | (0) | (100) | (0) | (100) | (0) | (100) | (2.7) | (97.3) |

### Serum Antibody Response

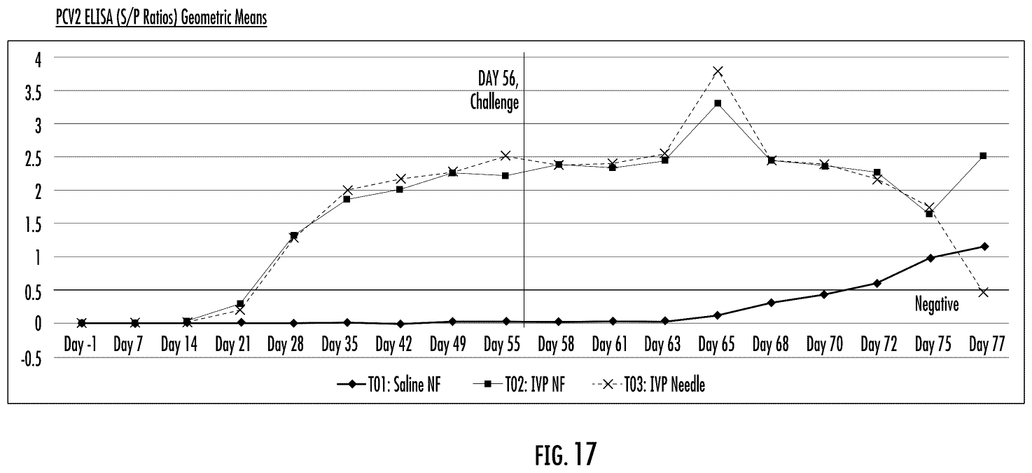

All treatment groups were seronegative to PCV2 prior to vaccination and showed an increase in PCV2 antibody titers post-challenge. The results are presented in Table 44 below, and in FIG. 17.

TABLE 44
PCV2 ELISA (S/P Ratios) By Treatment and Day of Study

| | PCV2 ELISA (S/P Ratios) Geometric Means | | | | | | | | | | | | | | | | | |
|---|---|---|---|---|---|---|---|---|---|---|---|---|---|---|---|---|---|---|
| | Day | Day | Day | Day | Day | Day | Day | Day | Day | Day | Day | Day | Day | Day | Day | Day | Day | Day |
| Trmnt | −1 | 7 | 14 | 21 | 28 | 35 | 42 | 49 | 55 | 58 | 61 | 63 | 65 | 68 | 70 | 72 | 75 | 77 |
| T01 | 0.012 | 0.006 | 0.010 | 0.008 | −0.004 | 0.008 | −0.003 | 0.020 | 0.029 | 0.024 | 0.030 | 0.031 | 0.129 | 0.319 | 0.438 | 0.608 | 0.993 | 1.157 |
| (Saline | | | | | | | | | | | | | | | | | | |
| NF) | | | | | | | | | | | | | | | | | | |
| T02 | 0.007 | 0.008 | 0.036 | 0.308 | 1.329 | 1.856 | 2.009 | 2.246 | 2.222 | 2.368 | 2.345 | 2.452 | 3.308 | 2.451 | 2.370 | 2.268 | 1.639 | 2.513 |
| (IVP | | | | | | | | | | | | | | | | | | |
| NF) | | | | | | | | | | | | | | | | | | |
| T03 | 0.008 | 0.006 | 0.022 | 0.208 | 1.289 | 2.014 | 2.178 | 2.285 | 2.519 | 2.374 | 2.406 | 2.546 | 3.795 | 2.437 | 2.396 | 2.173 | 1.752 | 2.388 |
| (IVP | | | | | | | | | | | | | | | | | | |
| Needle) | | | | | | | | | | | | | | | | | | |

*S/P Ratio: positive if ≥0.5.

PCV2 Viremia (ddPCR)

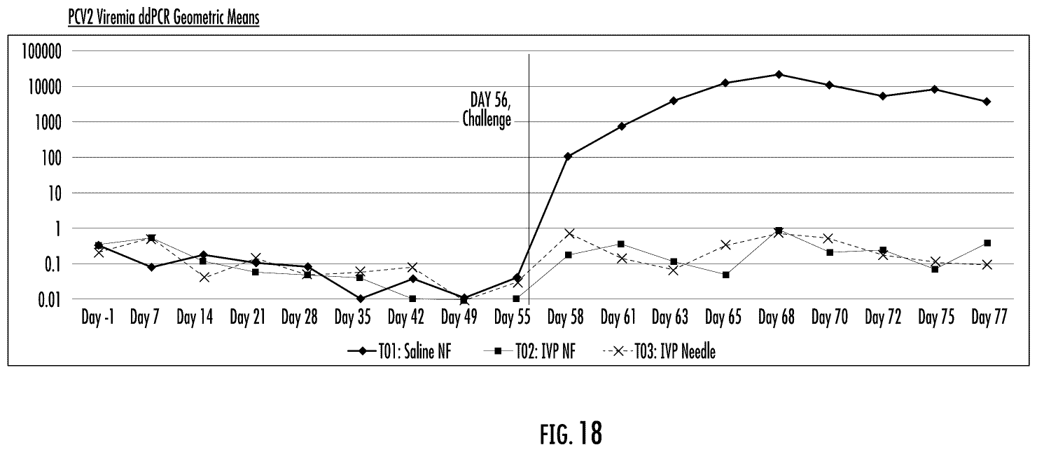

Table 45 and FIG. 18 summarize the viremia results. All animals remain free of both PCV2 viremia prior to challenge, thus validating the study. The results of the ddPCR assay for “PCV2 viremia ever present” are presented in Tables 46 and 47. The viremia results support 5 that both T02 (Needle-free) and T03 (Needle) were significantly different than the control group.

TABLE 45
PCV2 Viremia (Geometric Means Copies per 20 μL sample)
by Treatment Group and Study Day as Detected by ddPCR

| | Geometric Means PCV2 Viremia | | | | | | | | | | |
|---|---|---|---|---|---|---|---|---|---|---|---|
| | Day | Day | Day | Day | Day | Day | Day | Day | Day | Day | Day |
| Trmnt | −1 | 7 | 14 | 21 | 28 | 35 | 42 | 49 | 55 | 58 | 61 |
| T01 (Saline NF) | 0.34 | 0.08 | 0.18 | 0.11 | 0.08 | 0.01 | 0.04 | 0.01 | 0.04 | 96.38 | 725.93 |
| T02 (IVP NF) | 0.33 | 0.49 | 0.11 | 0.06 | 0.05 | 0.04 | 0.01 | 0.01 | 0.01 | 0.18 | 0.36 |
| T03 (IVP Needle) | 0.21 | 0.50 | 0.04 | 0.15 | 0.05 | 0.06 | 0.08 | 0.00 | 0.03 | 0.70 | 0.15 |

| | Geometric Means PCV2 Viremia | | | | | | |
|---|---|---|---|---|---|---|---|
| | Day | Day | Day | Day | Day | Day | Day |
| Trmnt | 63 | 65 | 68 | 70 | 72 | 75 | 77 |
| T01 (Saline NF) | 3902.44 | 12118.67 | 21782.36 | 10929.05 | 5514.59 | 8224.42 | 3654.45 |
| T02 (IVP NF) | 0.11 | 0.05 | 0.85 | 0.21 | 0.24 | 0.07 | 0.37 |
| T03 (IVP Needle) | 0.07 | 0.34 | 0.71 | 0.51 | 0.18 | 0.11 | 0.09 |

Viremia is PCV2 positive if result ≥11 copies per 20 μL sample.

TABLE 46
PCV2 Viremia Ever Present [Number of Animals (Percent of Animals)] by Treatment Group and Study Day as Detected by ddPCR

| | PCV2 Viremia Ever Present | | | | | | | | | | | | | | | | | |
|---|---|---|---|---|---|---|---|---|---|---|---|---|---|---|---|---|---|---|
| | Day | Day | Day | Day | Day | Day | Day | Day | Day | Day | Day | Day | Day | Day | Day | Day | Day | Day |
| Trmnt | −1 | 7 | 14 | 21 | 28 | 35 | 42 | 49 | 55 | 58 | 61 | 63 | 65 | 68 | 70 | 72 | 75 | 77 |
| T01 (Saline NF) | 0 | 0 | 0 | 0 | 0 | 0 | 0 | 0 | 0 | 37 | 37 | 37 | 37 | 37 | 36 | 35 | 36 | 36 |
| | (0) | (0) | (0) | (0) | (0) | (0) | (0) | (0) | (0) | (100) | (100) | (100) | (100) | (100) | (100) | (97.2) | (100) | (100) |
| T02 (IVP NF) | 0 | 0 | 0 | 0 | 0 | 0 | 0 | 0 | 0 | 0 | 1 | 0 | 0 | 1 | 0 | 1 | 0 | 3 |
| | (0) | (0) | (0) | (0) | (0) | (0) | (0) | (0) | (0) | (0) | (2.6) | (0) | (0) | (2.7) | (0) | (2.7) | (0) | (8.1) |
| T03 (IVP Needle) | 0 | 0 | 0 | 0 | 0 | 0 | 0 | 0 | 0 | 2 | 0 | 0 | 1 | 0 | 0 | 0 | 0 | 0 |
| | (0) | (0) | (0) | (0) | (0) | (0) | (0) | (0) | (0) | (6.1) | (0) | (0) | (3) | (0) | (0) | (0) | (0) | (0) |

TABLE 47
PCV2 Viremia Ever Present [# of Animals (% of
Animals)] Summaries as Detected by ddPCR

| | | | Significance Comparison | | |
|---|---|---|---|---|---|
| | Positive | Negative | | | Significance |
| Trmnt | # (%) | # (%) | Comparison | P-Value | (P ≤ 0.10) |
| T01 (Saline NF) | 37 (94.9) | 2 (5.1) | | | |
| T02 (IVP NF) | 6 (16.2) | 31 (83.8) | vs. T01 | 0.0094 | Yes |
| T03 (IVP Needle) | 3 (8.1) | 34 (91.9) | vs. T01 | 0.0070 | Yes |
| | | | vs. T02 | 0.4171 | Not |
| | | | | | Significant |

Fecal Shedding (ddPCR)

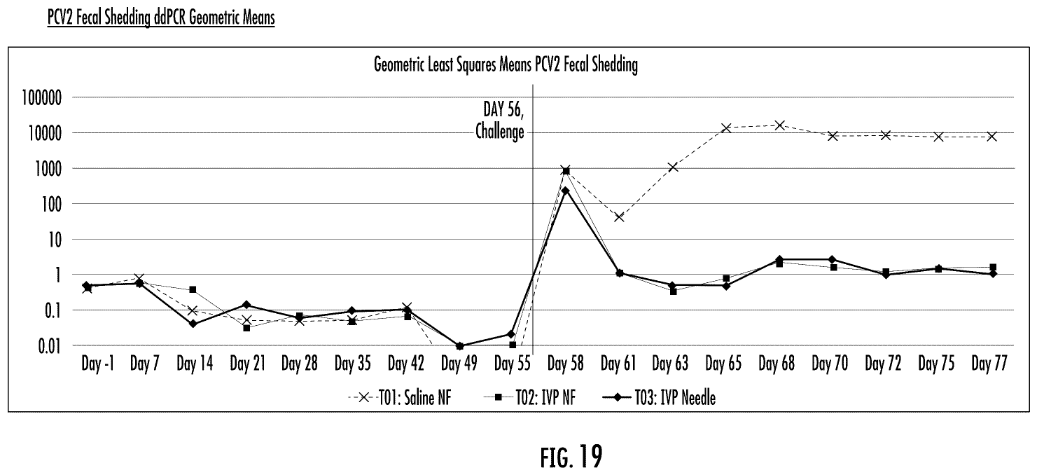

The fecal shedding results are presented in Tables 48, 49, and 51 below, and in FIG. 19.

TABLE 48
PCV2 Fecal Shedding (Geometric Means Copies per 20 μL
sample) by Treatment Group and Study Day as Detected by ddPCR

| | Geometric Means PCV2 Fecal Shedding | | | | | | | | | | |
|---|---|---|---|---|---|---|---|---|---|---|---|
| | Day | Day | Day | Day | Day | Day | Day | Day | Day | Day | Day |
| Trmnt | −1 | 7 | 14 | 21 | 28 | 35 | 42 | 49 | 55 | 58 | 61 |
| T01 (Saline NF) | 0.38 | 0.79 | 0.09 | 0.05 | 0.05 | 0.05 | 0.11 | 0.00 | 0.00 | 918.39 | 37.40 |
| T02 (IVP NF) | 0.43 | 0.54 | 0.34 | 0.03 | 0.07 | 0.05 | 0.07 | 0.01 | 0.01 | 847.29 | 1.22 |
| T03 (IVP Needle) | 0.47 | 0.56 | 0.04 | 0.14 | 0.06 | 0.09 | 0.10 | 0.01 | 0.02 | 244.14 | 1.03 |

| | Geometric Means PCV2 Fecal Shedding | | | | | | |
|---|---|---|---|---|---|---|---|
| | Day | Day | Day | Day | Day | Day | Day |
| Trmnt | 63 | 65 | 68 | 70 | 72 | 75 | 77 |
| T01 (Saline NF) | 1021.57 | 13121.87 | 14959.74 | 7964.23 | 8035.88 | 7355.92 | 7140.47 |
| T02 (IVP NF) | 0.37 | 0.83 | 2.36 | 1.61 | 1.16 | 1.41 | 1.50 |
| T03 (IVP Needle) | 0.50 | 0.49 | 2.59 | 2.58 | 0.96 | 1.44 | 1.01 |

Fecal shedding is PCV2 positive if result ≥45 per 20 μL sample.

TABLE 49
Fecal Shedding Ever Present [Number of Animals (Percent of Animals)] by Treatment Group and Study Day as Detected by ddPCR

| | PCV2 Fecal Shedding Ever Present | | | | | | | | | | | | | | | | | |
|---|---|---|---|---|---|---|---|---|---|---|---|---|---|---|---|---|---|---|
| | Day | Day | Day | Day | Day | Day | Day | Day | Day | Day | Day | Day | Day | Day | Day | Day | Day | Day |
| Trmnt | −1 | 7 | 14 | 21 | 28 | 35 | 42 | 49 | 55 | 58 | 61 | 63 | 65 | 68 | 70 | 72 | 75 | 77 |
| T01 | 0 | 0 | 0 | 0 | 0 | 0 | 0 | 0 | 0 | 36 | 16 | 34 | 37 | 37 | 36 | 35 | 36 | 36 |
| (Saline | (0) | (0) | (0) | (0) | (0) | (0) | (0) | (0) | (0) | (97.3) | (43.2) | (91.9) | (100) | (100) | (100) | (97.2) | (100) | (100) |
| NF) | | | | | | | | | | | | | | | | | | |
| T02 | 0 | 0 | 0 | 0 | 0 | 0 | 0 | 0 | 0 | 36 | 0 | 0 | 0 | 1 | 0 | 0 | 0 | 0 |
| (IVP NF) | (0) | (0) | (0) | (0) | (0) | (0) | (0) | (0) | (0) | (94.7) | (0) | (0) | (0) | (2.7) | (0) | (0) | (0) | (0) |
| T03 | 0 | 0 | 0 | 0 | 0 | 0 | 0 | 0 | 0 | 26 | 1 | 0 | 0 | 1 | 2 | 0 | 0 | 0 |
| (IVP | (0) | (0) | (0) | (0) | (0) | (0) | (0) | (0) | (0) | (78.7) | (3) | (0) | (0) | (3) | (6.1) | (0) | (0) | (0) |
| Needle) | | | | | | | | | | | | | | | | | | |

TABLE 50
PCV2 Fecal Shedding Ever Present Summaries as Detected by ddPCR

| | | | Significance Comparison | | |
|---|---|---|---|---|---|
| | Positive | Negative | | | Significance |
| Trmnt | # (%) | # (%) | Comparison | P-Value | (P ≤ 0.10) |
| T01 (Saline NF) | 37 (94.9) | 2 (5.1) | | | |
| T02 (IVP NF) | 35 (94.6) | 2 (5.4) | vs. T01 | 0.0094 | Not tested |
| T03 (IVP Needle) | 26 (70.3) | 11 (29.7) | vs. T01 | 0.0070 | Not tested |
| | | | vs. T02 | 0.4171 | Not tested |

Nasal Shedding (ddPCR)

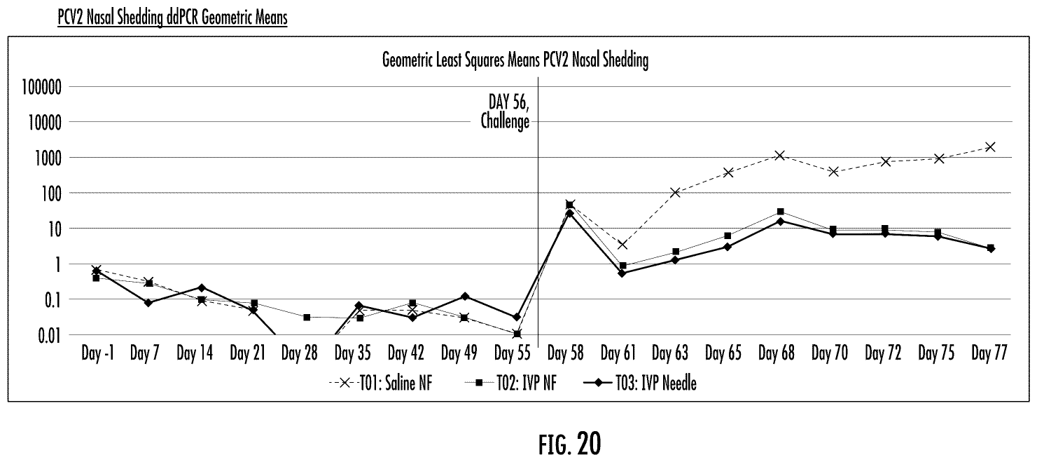

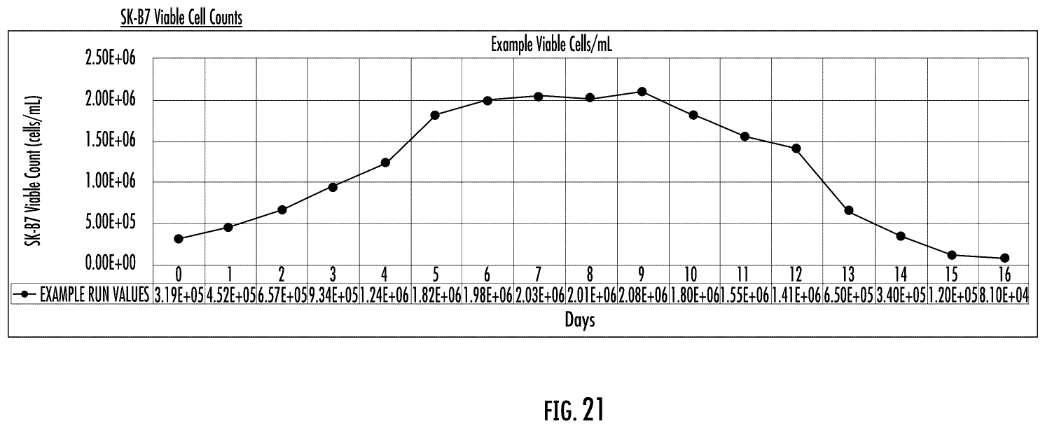

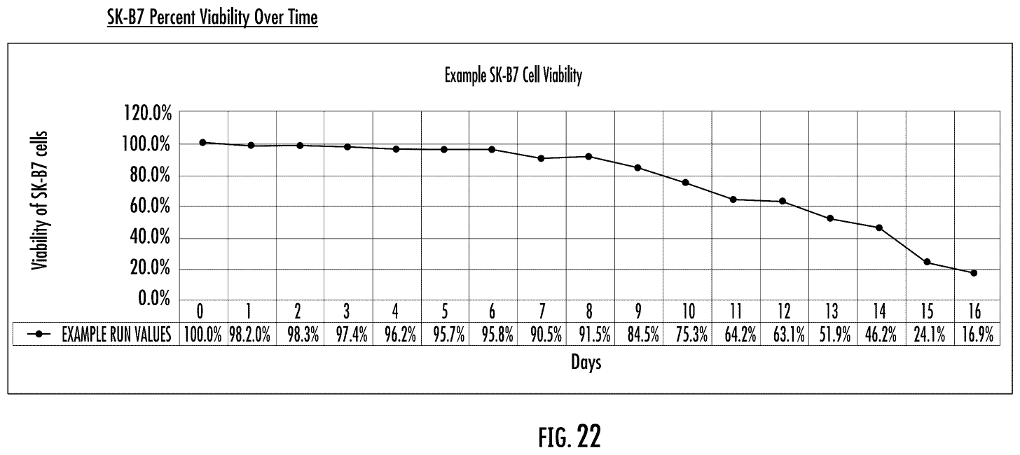

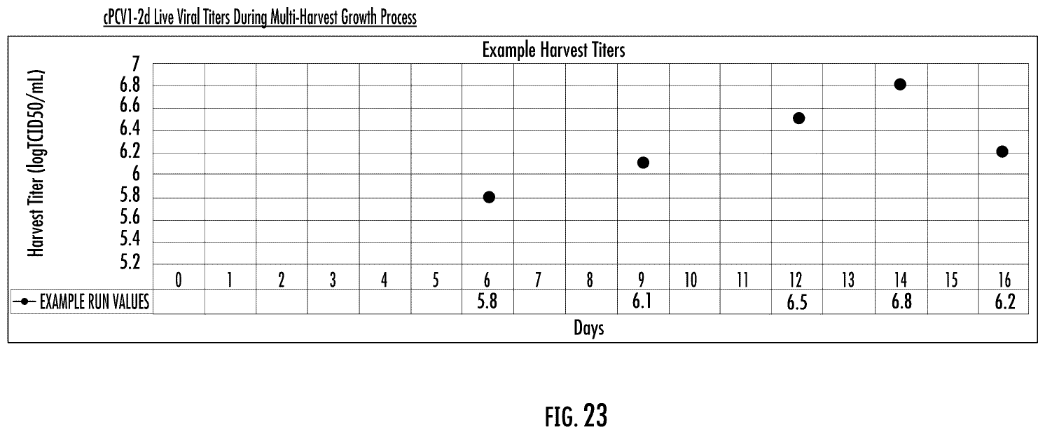

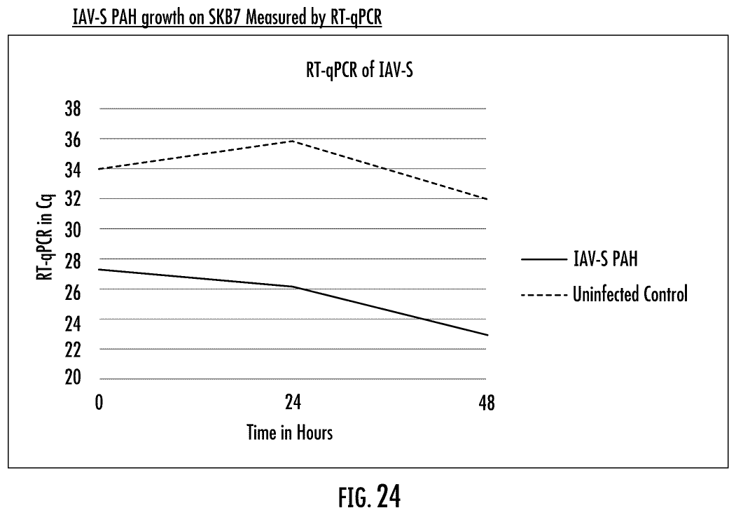

The nasal shedding results are presented in Tables 51, 52, and 53 below, and in FIG. 20.

TABLE 51
PCV2 Nasal Shedding (Geometric Means Copies per 20 μL
sample) by Treatment Group and Study Day as Detected by ddPCR

| | Geometric Means PCV2 Nasal Shedding | | | | | | | | | | |
|---|---|---|---|---|---|---|---|---|---|---|---|
| | Day | Day | Day | Day | Day | Day | Day | Day | Day | Day | Day |
| Trmnt | −1 | 7 | 14 | 21 | 28 | 35 | 42 | 49 | 55 | 58 | 61 |
| T01 (Saline NF) | 0.68 | 0.31 | 0.09 | 0.05 | 0.00 | 0.05 | 0.05 | 0.03 | 0.01 | 49.11 | 3.41 |
| T02 (IVP NF) | 0.41 | 0.28 | 0.10 | 0.08 | 0.03 | 0.03 | 0.08 | 0.03 | 0.01 | 46.23 | 0.84 |
| T03 (IVP Needle) | 0.63 | 0.08 | 0.20 | 0.05 | 0.00 | 0.07 | 0.03 | 0.12 | 0.03 | 30.36 | 0.53 |

| | Geometric Means PCV2 Nasal Shedding | | | | | | |
|---|---|---|---|---|---|---|---|
| | Day | Day | Day | Day | Day | Day | Day |
| Trmnt | 63 | 65 | 68 | 70 | 72 | 75 | 77 |
| T01 (Saline NF) | 99.46 | 371.90 | 1141.43 | 384.55 | 731.74 | 917.86 | 1994.74 |
| T02 (IVP NF) | 2.16 | 6.33 | 29.70 | 8.99 | 8.98 | 7.59 | 2.58 |
| T03 (IVP Needle) | 1.33 | 3.07 | 16.45 | 7.00 | 7.14 | 6.06 | 2.73 |

Oronasal Shedding is PCV2 positive if result ≥11 copies per 20 μL sample.

TABLE 52
Nasal Shedding Ever Present [Number of Animals (Percent of Animals)]
by Treatment Group and Study Day as Detected by ddPCR

| | PCV2 Nasal Shedding Ever Present | | | | | | | | | | |
|---|---|---|---|---|---|---|---|---|---|---|---|
| | Day | Day | Day | Day | Day | Day | Day | Day | Day | Day | Day |
| Trmnt | −1 | 7 | 14 | 21 | 28 | 35 | 42 | 49 | 55 | 58 | 61 |
| T01 (Saline NF) | 0 | 0 | 0 | 0 | 0 | 0 | 0 | 0 | 0 | 34 | 6 |
| | (0) | (0) | (0) | (0) | (0) | (0) | (0) | (0) | (0) | (91.9) | (16.2) |
| T02 (IVP NF) | 0 | 0 | 0 | 0 | 0 | 0 | 0 | 0 | 0 | 28 | 0 |
| | (0) | (0) | (0) | (0) | (0) | (0) | (0) | (0) | (0) | (75.7 | (0) |
| T03 (IVP Needle) | 0 | 0 | 0 | 0 | 0 | 0 | 0 | 0 | 0 | 29 | 0 |
| | (0) | (0) | (0) | (0) | (0) | (0) | (0) | (0) | (0) | (87.9) | (0) |

| | PCV2 Nasal Shedding Ever Present | | | | | | |
|---|---|---|---|---|---|---|---|
| | Day | Day | Day | Day | Day | Day | Day |
| Trmnt | 63 | 65 | 68 | 70 | 72 | 75 | 77 |
| T01 (Saline NF) | 36 | 37 | 37 | 36 | 36 | 36 | 35 |
| | (97.3) | (100) | (100) | (100) | (100) | (100) | (97.2) |
| T02 (IVP NF) | 2 | 11 | 32 | 21 | 14 | 13 | 6 |
| | (5.4) | (29.7) | (86.5) | (56.8) | (37.8) | (35.1) | (16.2) |
| T03 (IVP Needle) | 0 | 4 | 18 | 15 | 7 | 10 | 6 |
| | (0) | (12.1) | (54.5) | (45.5) | (21.2) | (30.3) | (18.2) |

TABLE 53
PCV2 Nasal Shedding Ever Present Summaries as Detected by ddPCR

| | | | Significance Comparison | | |
|---|---|---|---|---|---|
| | Positive | Negative | | | Significance |
| Trmnt | # (%) | # (%) | Comparison | P-Value | (P ≤ 0.10) |
| T01 (Saline NF) | 37 (94.9) | 2 (5.1) | | | |
| T02 (IVP NF) | 37 (100) | 0 (0) | vs. T01 | 0.4937 | Not significant |
| T03 (IVP Needle) | 33 (89.2) | 4 (10.8) | vs. T01 | 0.4246 | Not significant |
| | | | vs. T02 | 0.1148 | Not significant |

### Tissues

The tissue results are presented in Tables 54, and 55 below.

TABLE 54
Individual Tissue Scores by Tissue and Parameter

| | | Number of Animals for Each Score (0-3) by Treatment Group | | | | | | | | | | | |
|---|---|---|---|---|---|---|---|---|---|---|---|---|---|
| | | T01 (Saline NF) | | | | T02 (IVP NF) | | | | T03 (IVP Needle) | | | |
| | | 0 | 1 | 2 | 3 | 0 | 1 | 2 | 3 | 0 | 1 | 2 | 3 |
| Lymphoid | MLN | 30 | 5 | 1 | 1 | 37 | 0 | 0 | 0 | 33 | 0 | 0 | 0 |
| Depletion | SLN | 6 | 23 | 6 | 2 | 27 | 8 | 2 | 0 | 13 | 18 | 2 | 0 |
| (LD) | TLN | 16 | 15 | 5 | 1 | 36 | 1 | 0 | 0 | 31 | 2 | 0 | 0 |
| | Tonsil | 23 | 9 | 4 | 0 | 33 | 4 | 0 | 0 | 31 | 2 | 0 | 0 |
| Histiocytic | MLN | 31 | 5 | 0 | 1 | 37 | 0 | 0 | 0 | 33 | 0 | 0 | 0 |
| Replacement | SLN | 27 | 7 | 2 | 1 | 36 | 1 | 0 | 0 | 33 | 0 | 0 | 0 |
| (HR) | TLN | 27 | 9 | 0 | 1 | 37 | 0 | 0 | 0 | 33 | 0 | 0 | 0 |
| | Tonsil | 20 | 10 | 4 | 2 | 37 | 0 | 0 | 0 | 32 | 1 | 0 | 0 |
| IHC | MLN | 20 | 11 | 5 | 1 | 34 | 1 | 1 | 1 | 30 | 3 | 0 | 0 |
| | SLN | 23 | 7 | 4 | 3 | 36 | 0 | 1 | 0 | 32 | 1 | 0 | 0 |
| | TLN | 17 | 6 | 9 | 5 | 34 | 2 | 1 | 0 | 33 | 0 | 0 | 0 |
| | Tonsil | 1 | 4 | 7 | 25 | 36 | 0 | 1 | 0 | 33 | 0 | 0 | 0 |

TABLE 55
Tissue Score Evaluation

| | | Normal | | Abnormal | | Significance Comparison | | |
|---|---|---|---|---|---|---|---|---|
| | | (0) | | (1, 2, 3) | | | | Significance |
| | | # (%) | | # (%) | | Comparison | P-Value | (P ≤ 0.10) |
| Lymphoid | T01 (Saline NF) | 4 | (10.8) | 33 | (89.2) | | | |
| Depletion | T02 (IVP NF) | 23 | (62.2) | 14 | (37.8) | vs. T01 | 0.0172 | Yes |
| (LD) | T03 (IVP Needle) | 11 | (33.3) | 22 | (66.7) | vs. T01 | 0.1038 | Not |
| | | | | | | | | significant |
| | | | | | | vs. T02 | 0.0965 | Yes |
| Histiocytic | T01 (Saline NF) | 15 | (40.5) | 22 | (59.5) | | | |
| Replacement | T02 (IVP NF) | 36 | (97.3) | 1 | (2.7) | vs. T01 | 0.0252 | Yes |
| (HR) | T03 (IVP Needle) | 32 | (97.0) | 1 | (3.0) | vs. T01 | 0.0280 | Yes |
| | | | | | | vs. T02 | 0.9359 | Not |
| | | | | | | | | significant |
| LD and/ | T01 (Saline NF) | 3 | (8.1) | 34 | (91.9) | | | |
| or HR | T02 (IVP NF) | 23 | (62.2) | 14 | (37.8) | vs. T01 | 0.0216 | Yes |
| | T03 (IVP Needle) | 11 | (33.3) | 22 | (66.7) | vs. T01 | 0.0953 | Yes |
| | | | | | | vs. T02 | 0.1461 | Not |
| | | | | | | | | significant |
| IHC | T01 (Saline NF) | 1 | (2.7) | 36 | (97.3) | | | |
| | T02 (IVP NF) | 34 | (91.9) | 3 | (8.1) | vs. T01 | 0.0091 | Yes |
| | T03 (IVP Needle) | 29 | (87.9) | 4 | (12.1) | vs. T01 | 0.0106 | Yes |

| | | |
|---|---|---|
| vs. T02 | 0.6997 | Not |
| | | significant |

### CONCLUSIONS

No injection site reactions were observed in in animals receiving the IVP when administered intramuscularly via a needle (T03) or a needle-free device (T02).

A cPCV1-2d vaccine formulated at 4.1 log 10/0.5 mL dose was efficacious versus a saline control when administered with a needle or a needle-free device.

T02 (Needle-free) was significantly different than controls for viremia, lymphoid depletion, histiocytic replacement, lymphoid depletion and/or histiocytic replacement, and IHC.

T03 (Needle) was significantly different than controls for viremia, histiocytic replacement, histiocytic replacement and/or lymphoid depletion, and IHC.

T02 (Needle-free) was significantly different than T03 (Needle) for lymphoid depletion.

Neither the Needle-free or Needle were significantly different than controls for fecal shedding nor nasal shedding. Future studies should be powered higher for showing significance. Nasal swabs should be replaced with oronasal swabs to increase the level of shedding and for showing significance.

Needle-free is an appropriate method of administration moving forward.

### Example 6-Growth of PCV1-2a, PCV1-2b, and PCV2d on PERV Negative SKB7 and SKD8 Cells

SKB7 and SKD8 cells were infected with PCV1-2a, PCV1-2b, and PCV1-2d chimeric viruses and PCV2d field virus to confirm the edited cell clones could support growth of these virus stocks.

### Materials

SKB7 cells & SKD8 cells
cPCV1-2d pre-master seed virus (SEQ ID NO:16)
cPCV1-2a working seed virus (SEQ ID NO: 24)
cPCV1-2b working seed virus (SEQ ID NO: 25)
PCV2d field virus (SEQ ID NO: 22)
Optimem with 0.25% LAH (Zoetis)
Fetal bovine serum (Zoetis)
100× GlutaMAX, (Gibco cat #35050061)
Gentamicin, 50 mg/mL (Gibco part #15750060)
Antibiotic/antimycotic, 100× (Gibco cat #15240096)
Growth Media: Optimem with 0.25% LAH with 2% FBS, 1× GlutaMAX, 20 ug/ml gentamicin, 1×antibiotic/antimycotic.
6-well Costar plates
5D5-5H4 anti PCV2 Mab lot #2256-43-050405 (Zoetis)
AlexaFluor 488 AffiniPure Goat Anti-Mouse IgG (H+L) (Jackson ImmunoResearch115-545-003)
80% Acetone

### Method:

Planted one 6-well plate of SKB7 and one 6-well plate of SKD8 cells at 0.4×10⁶ cells/well in Growth media.

Added the following to each well:
Well 1:0.1 MOI cPCV1-2a virus stock (SEQ ID NO: 24)
Well 2:0.1 MOI cPCV1-2b virus stock (SEQ ID NO: 25)
Well 3:0.1 MOI cPCV1-2d virus stock (SEQ ID NO: 16)
Well 4:0.1 MOI PCV2d field virus (SEQ ID NO: 22)
Well 5: Growth media without virus

5 days post infection, the supernatant was removed, and cells were fixed with 80% acetone for 15 minutes.

Acetone was removed and plates were air-dried for 15 minutes.

Wells were stained with 1:1000 dilution of 5D5-5H4 anti PCV2 Mab diluted in PBS. Used 1 mL per well of 6-well plate and incubated 37° C. for 1 hour.

Wells were washed 3× with PBS and stained with 1:100 dilution of Alexa Fluor 488-conjugated Affin pure goat anti-mouse IgG (H+L). Used 1 mL of conjugate per well of 6-well plate and incubated at 37° C. for 1 hour. Wells were washed 3× with PBS and then visualized under a fluorescent microscope.

Scoring Method: Infected wells were given a score of + to +++based on percentage of the well that was infected with virus, as determined by fluorescent staining. A+score indicates 10 to 25% of the well was fluorescent, a ++score indicates 26 to 50% of the well was fluorescent, a +++indicates 51 to 75% of the well was fluorescent and a ++++would indicate that 76-100% of the cells in the well were fluorescent. The—score indicates there was no fluorescent stainning in the well.

TABLE 56
Percent of Infected Cells on SK-B7 and SK-D8 Cell Lines

| Virus Used for Infection | SK-B7 Cells | SK-D8 Cells |
|---|---|---|
| PCV1-2d chimera | ++ | ++ |
| PCV1-2a chimera | +++ | +++ |
| PCV1-2b chimera | +++ | +++ |
| PCV2d | ++ | + |
| No virus, negative control | − | − |

+ = 10-25% of cells infected
++ = 26-50% cells infected
+++ = 51-75% cells infected
++++ = 76-100% cells infected
− = no cells infected

### Conclusions:

SKB7 and SKD8 cells are able to support the growth of PCV1-2a chimeric virus, PCV1-2b chimeric virus, PCV1-2d chimeric virus and PCV2d field virus. This is evidenced by the positive IFA staining seen with both SKB7 and SKD8 cells as represented in Table 56. In contrast, the mock infected SKB7 and SKD8 cells showed negative IFA staining.

### Example 7 Growth of Influenza A Virus, Swine (IAV-S) on PERV negative SKB7 cells

### Materials

SKB7 cells
Optimem with 0.25% LAH (Zoetis)
Fetal bovine serum (Zoetis)
100× GlutaMAX, (Gibco cat #35050061)
Anti-Anti is Antibiotic/antimycotic, 100× (Gibco cat #15240096)
Gentamicin, 50 mg/mL (Gibco part #15750060)
Bovine rota corona trypsin (Zoetis)
6-well plates (Costar #3516)
SIV H1N1 PAH618 virus (GenBank Accession NO: MT377725.1)
Pathogen nucleic acid extraction kit (Indical Bioscience #SP54104)
Anti-influenza A antibody (MilliporeSigma MSxInfluenzaA Mab8257)
AlexaFluor 488 AffiniPure Goat Anti-Mouse IgG (H+L) (Jackson ImmunoResearch 115-545-003)

Growth Media: Optimem with 0.25% LAH with 2% FBS, 1× GlutaMAX, 20 ug/ml gentamicin, 1×antibiotic/antimycotic.

Infection Media: Optimem with 0.25% LAH with 2% FBS, 1× GlutaMAX, 20 ug/ml gentamicin, 1×antibiotic/antimycotic, 0.3% bovine corona trypsin

### Method:

Infection with Influenza a Virus (IAV-S) PAH618:
1. Plate SKB7 cells on 1×6-well plate plates with 3×10\hat{ }5 cells per well and 2 mL media per well (1.5×10\hat{ }5 cells/ml).
2. Incubate at 37 C, 5% CO₂ until wells are confluent (approximately 1.2×10\hat{ }6 cells/well) 2-3 days
3. Remove media and gently rinse wells with PBS.
4. Make infection media with 0.3% trypsin. Add 2 mL infection media (with trypsin) to 2 wells to act as negative control (uninfected) wells.
5. Add PAH618 to media at an MOI of 0.001. Starting titer of PAH618 is 7 log TCID50.
a. For each plate, make 12 mL of media
b. Assuming there are 1.2×10\hat{ }6 cells per confluent well×5 wells to be infected
c. Multiply # of cells by MOI of 0.001 to give a total # of viral particles needed of 6000/plate
d. Virus as a Log TCID50 of 7, 10\hat{ }7 TCID50, 6000 particles/10\hat{ }7 TCID50/mL=0.0006 ml virus=0.6 μl virus per 10 mL of media (enough for 5 wells)
6. Add 2 mL media with virus to remaining 4 wells.
7. Incubate plates at 37 C, 5% CO₂ for 2 days.
8. Take 0.5 mL sample from an infected well and uninfected well at 0 hrs, 24 hrs, 48 hrs post infection.
9. After 2 day incubation, fix plates with ice-cold 80% acetone for 15 minutes and allow to dry for 15 minutes.
10. Add mouse Mab diluted at 1:100 with PBS and incubate plate for 1 hour a 37 C.
11. Add AlexaFluor 488 diluted 1:100 with PBS and incubate plate for 1 hour at 37 C.
12. Image plates with Cytation2 5 or observe infection with another fluorescent microscope.
   RNA Purification and RT qPCR:

### Materials

IndiSpin Pathogen Kit (Indical SP54104)
IAV-S RT-qPCR Reagents

PAH618 Forward primer

(SEQ ID NO: 26)

5′ ATCTATTCAACGGTCGCCAG 3′, 100 μM stock
PAH618 Reverse primer

(SEQ ID NO: 27)

5′ GGATGTGCTCTAATGGGTCG 3′, 100 μM stock
PAH618 Probe

(SEQ ID NO: 28)

5′ TTGGTACTGGTAGTCTCCCTGGGG 3′, 100 μM stock

iTaq Universal Probes One-Step Kit (Bio-Rad 172-5141)
Standard Curve IAV-S RNA, which contains 1e6 copies per 5 ul
Nuclease-Free Water (Ambion AM9937)
Bio-Rad CFX384 Thermocycler with CFX Manager Software
Biomek 3000/4000
384-Well White PCR Plates (Bio-Rad HSP3805)
Microseal B Film (Bio-Rad MSB1001)
Clear U-Bottom 96-Well Plates (Greiner Bio-One 650101)
Quarter module reservoirs, divided by length (Beckman 372788)
General Lab Supplies (pipettes, tips, etc).

### Method

1. Purify RNA from the samples using the Indical IndiSpin Pathogen kit.
2. Test each sample for viral RNA via qPCR using the PAH618 primer/probe

### RTqPCR:

1. In one side of a quarter-module reservoir divided by length, prepare RT-qPCR reagents according to the IAV-S RT-qPCR Excel template.
2. In the other side of the quarter-module reservoir, add 3 mL of nuclease-free water.
3. Add 50-100 uL of each RNA sample to a Greiner clear U-bottom 96-well plate. Add sample 1 to well A1, sample 2 to well B1, etc. Samples are arranged in columns, not rows. 4. Thaw one vial of IAV-S RNA standard. Add 50-100 ul to well A1 of a Greiner 96-well plate.
5. Run the method entitled “384-Well PCR Plate Loading” (most recent version) on the Biomek 3000/4000 in lab 207.1.
1. Use Biomek method 384 well PCR plate prep Qiacube HT
2. Calibrate x and y axis on Biomeck before use.
3. Load materials onto Biomek following protocol-change number of samples
4. Under instrument set up make sure correct number of tips are available (matching tip box)
6. Seal the 384-well plate with Microseal B Film.
7. Centrifuge the plate to collect fluid to the bottom of the plate.
8. Load the plate into the CFX384 Thermocycler. Analyze using the SIV_MLV protocol.
9. Export data to a .csv file and analyze as required using the RT-qPCR Data Analysis Template.

### Results:

Table 57 represents the percentage of SKB7 cells in the 6-well plate infected with IAV-S PAH618 or negative control. Plates were read on the Cytation5 instrument. Between 51-75% of SKB7 cells were infected with IAV-S PAH 618 while no SKB7 cells were infected with IAV-S PAH 618.

TABLE 57
Percentage of Infected SKB7 Cells

| Virus Infected | SKB7 Fluorescent Cells |
|---|---|
| IAV-S PAH618 | +++ |
| No virus, negative control | − |

+ = 10-25% of cells infected
++ = 26-50% cells infected
+++ = 51-75% cells infected
++++ = 76-100% cells infected
− = no cells infected

Cq of IAV-S PAH 618 virus decreased from 0 to 48 hours after infection, as measured by RT qPCR. A decrease in the Cq value indicates the virus genome is increasing and the virus is growing.

TABLE 58
IAV-S RT-qPCR results

| | RT-qPCR Cq Values | | |
|---|---|---|---|
| Sample | 0 HR | 24 HR | 48 HR |
| IAV-S PAH | 27.28 | 26.13 | 22.95 |
| Uninfected Control | 34 | 35.79 | 31.95 |

The results in Table 59 confirm the IAV-S RT-qPCR assay ran correctly since the positive control (standards) gave the expected Cq values and the non-template control (NTC) was not recorded in the assay. The N/A designation indicates no IAV-S nucleic acid was present in the sample.

TABLE 59
RT-qPCR IAV-S Dilution Standard

| Sample | Target copies | Cq |
|---|---|---|
| Std 01 | 1,000,000 | 19.27 |
| Std 02 | 100,000 | 22.35 |
| Std 03 | 10,000 | 25.47 |
| Std 04 | 1,000 | 29.01 |
| Std 05 | 100 | 31.81 |
| Std 06 | 10 | 35.23 |
| Std 07 | 1 | 38.54 |
| NTC | 0 | N/A |

### Conclusions:

The SKB7 cell line can support the growth of IAV-S PAH 618 based on increased fluorescence in IAV-S PAH 618 infected SKB7 cells upon infection with 0.001 MOI of virus. It was observed that between 51-75% of SKB7 cells were infected with IAV-S PAH 618, while no SKB7 cells were infected (fluorescent) in mock-infected cells. In addition, the Cq of IAV-S PAH 618 virus decreased from 0 to 48 hours after infection, as measured by RT qPCR. A decrease in the Cq value indicates the virus genome is increasing (replicating) and the virus is growing.

### Example 8 Growth of BVDV-1 and BVDV-2 on PERV negative SKB7 cells

### Materials:

### Cell Lines:

Bovine turbinate cells
SKB7cells
BK6 cells

### Virus Strains:

BVD-1a strain 5960 cytopathic (cpBDV-1 strain 5960-National Animal Disease Center, United States Department of Agriculture, Ames, Iowa) (Similar to GenBank NC_001461)
BVDV 1b OK1794-1 (similar to GenBank KF835697)
BVD-2 strain C125 (GenBank MH806434)

### Antibodies:

Mab15C5: Recognizes all BVDV, diluted to 1:3000 in PBS prior to use.
AlexaFluor 488 AffiniPure Goat Anti-Mouse IgG (H+L) (Jackson ImmunoResearch cat #115-545-003) diluted 1:250 in PBS.

### Media and Reagents:

Optimem with 0.25% LAH (Zoetis) for SKB7 cell growth.
DMEM (Zoetis) for BT and BK6 cell growth.
Fetal bovine serum (Zoetis)
Horse serum (HS) (Gibco Cat #16050122)
100× GlutaMAX, (Gibco cat #35050061)
Gentamicin, 50 mg/mL (Gibco part #15750060)
Anti/anti: Antibiotic/antimycotic, 100× (Gibco cat #15240096)
SKB7 Growth Media: Optimem with 0.25% LAH supplemented with 2% FBS, 1× GlutaMAX, 20 ug/ml gentamicin, 1× antibiotic/antimycotic.
BT Growth Media: DMEM supplemented with 10% HS, 2× GlutaMAX, 20 ug/mL gentamicin, 1×anti-anti.
BT Titration Media: DMEM supplemented with 5% HS, 2× GlutaMAX, 20 ug/mL gentamicin, 1×anti-anti.
BK6 Growth Media: DMEM supplemented with 5% FBS, 2× GlutaMAX, 20 ug/mL gentamicin, 1×anti-anti.
Infection Media: DMEM media supplemented with 0.5% FBS, 2× GlutaMAX, 20 ug/mL gentamicin, 1× anti-anti.
T75 flasks
96 well plates (Corning Costar Cat #3585)
Assay Blocks, 2 mL (Costar Cat #3960)
80% Acetone

### Methods:

### Virus Growth:

Plant SKB7 cells into 4 × T75 flasks at 5×106 cells per flask with 35 mL SKB7 growth media.
Plant BK6 cells into 4 × T75 flasks at 5×106 cells per flask in 35 mL BK6 growth media
After 1 day, remove media from cells and wash once with Infection media.
Add the following to the flasks:

### SKB7 Flasks:

Flask 1:10 mL infection Media
Flask 2:0.1 MOI BVDV-1a 5960 in 10 mL infection media.
Flask 3:0.1 MOI BVDV-1b OK1794-1 in 10 mL infection media.
Flask 4:0.1 MOI BVDV-2 C125 in 10 mL infection media.

### BK6 Flasks:

Flask 1:10 mL infection Media
Flask 2:0.1 MOI BVDV-1a 5960 in 10 mL infection media.
Flask 3:0.1 MOI BVDV-1b OK1794-1 in 10 mL infection media.
Flask 4:0.1 MOI BVDV-2 C125 in 10 mL infection media.
Incubate flasks for 2 hours at 37° C. then add back 30 mL infection media.
Monitor flasks daily for signs of CPE
8 days post infection, cells were frozen at −80° C. overnight.

### Virus Titration:

Cells are planted 1 day prior to titration using 18K cells per 0.1 mL in each well. Thaw frozen flasks at 37° C. for 1 hr.

Place thawed flask contents in 50 ml conical and centrifuge 2500 rpm for 20 min to remove cell debris.

Titrate the thawed flasks in duplicate for each cell line and each virus.

Fill assay blocks with 900.0 uL/well of Titration media Load 100.0 uL of sample into columns 1 (initial 1:10 dilution) and 7.

Serially dilute 100 uL across the assay block—from columns 1 to 6 and columns 7 to 12

Once titrated, add 100.0 uL of desired dilution range from assay block to 96-well plate with BT cells planted 1 day prior.

Incubate plates at 37° C.+5% CO₂.

### IFA-Fixing/Staining Procedure

Four to five days post infection, dump plates and add 50-100 μL/well of 80% acetone. Let plates sit at room temperature for 10-15 minutes.

Dump fixative solution and rinse 3× with DI water.

Add 65-100 μL of antibody diluted in PBS. Incubate plates at 37° C.+5% CO₂ for 45 to 60 minutes.

Wash plates 3× with DI water.

Blot dry and store in foil until IFA read.

Read BVDV titration plates and calculate TCID50/mL of sample using Spearman-Karber method.

TABLE 60
Virus Titration (TCID50/mL) of BVDV Infected BK6 and
SKB7 Cell Lines at 0 and 8 Days Post-Infection

| | BK6 Cells | | SKB7 Cells | |
|---|---|---|---|---|
| Virus Used for Infection | Day 0 | Day 8 | Day 0 | Day 8 |
| BVDV-1a 5960 | ≤1.5 | 3.77 | ≤1.5 | 2.82 |
| BVDV-1b OK1794-1 | ≤1.5 | 6.12 | ≤1.5 | 3.10 |
| BVDV-2 C125 | ≤1.5 | 6.44 | ≤1.5 | 3.77 |
| No virus, negative control | ≤1.5 | ≤1.5 | ≤1.5 | ≤1.5 |

### BVDV RT-PCR

BVDV infected BK6 and SKB7 cells were used for BVDV RT-PCR to confirm the presence of BVDV RNA in the infected cells. This method was adapted from the Ridpath et al. 1998 method (Ridpath, J. F. and S. R. Bolin (1998). Differentiation of types 1a, 1b and 2 bovine viral diarrhoea virus (BVDV) by PCR. Mol Cell Probes 12 (2): 101-106).

### Materials:

QIAamp Viral RNA Mini Kit; Qiagen 52904
Absolute ethanol, 200 Proof, Fisher BP 2818-4
DNAse/RNAse free sterile microfuge tubes, Axygen MCT-175-C-S
RNA samples and controls purified by QIAamp Viral RNA Mini Kit
PCR thermowell reaction tubes Costar 6571
DNAse/RNAse free sterile barrier tips, Thermo Fisher Scientific-(10,20,200,1000)
Nuclease Free Water (NFW); Ambion AM9937
SuperScript One-Step RT-PCR kit, Invitrogen 10928-042
BVDV Ridpath-F (Forward Primer), 100 UM in TE; Sequence 5′ CAT GCC CAT AGT AGG AC 3′ (SEQ ID NO: 29)
BVDV Ridpath-R (Reverse Primer), 100 uM in TE; Sequence 5′ CCA TGT GCC ATG TAC AG 3′ (SEQ ID NO: 30)

### Extraction and Purification of Sample RNA

For each sample requiring RNA purification; perform the purification method according to manufacturer specifications for the purification of Viral RNA (spin protocol):
1. Add 560 μL of prepared Buffer AVL containing carrier RNA to bottom of a sterile 1.5 mL microfuge tube.
2. Add 140 ul of sample to Buffer AVL tube.
3. Mix with vortex (˜15s with pulse).
4. Incubate at 15-25° C. for 10 minutes.
5. Briefly centrifuge tubes to remove drops from lid.
6. Add 560 uL absolute ethanol, mix by vortex and briefly centrifuge to remove drops from lid.
7. Apply 630 μL of the above mixture to one QIAamp mini spin column in 2 mL collection tube.
8. Centrifuge at 6000 × g for 1 minute.
9. Remove spin column, discard collection tube and place spin column in new collection tube.
10. Repeat steps 7-9 to apply the entire sample preparation volume.
11. Add 500 uL wash buffer 1 (QIAamp solution AW1 supplemented with ethanol per manual instruction), change tips between samples.
12. Centrifuge at 6000 × g for 1 minute.
13. Remove spin column, discard collection tube and place spin column in new collection tube.
14. Add 500 uL wash buffer 2 (QIAamp solution AW2 supplemented with ethanol per manual instruction), change tips between samples.
15. Centrifuge at 20,000×g for 3 minutes.
16. Remove spin column, discard collection tube and place spin column in sterile 1.5 mL microfuge tube.
17. Add 60 μL of Buffer AVE elution buffer (QIAamp solution AVE) and incubate at room temperate for 1 minute.
18. Centrifuge at 6000 × g for 1 minute.
19. Remove spin column and discard. Cap microfuge tube with eluted DNA and store at ≤−20° C.

### Preparation of One-Step Real-time RT-PCR Master Mix

1. Completely thaw primers, buffer and reaction components. Mix all reagents and enzymes by moderate vortex and collect by brief centrifugation within a microcentrifuge, if necessary. Maintain on ice.
2. Combine each of the following assay components on ice in the order indicated. Use a tube large enough to handle the final volume. Add an additional ˜10% to the calculation to account for volume loss:

TABLE 61
Reagents and Volume per Reaction

| | Volume for 1 |
|---|---|
| Reagent | Reaction in ul |
| 2X Reaction Mix | 25.0 |
| Forward Primer (100 uM) | 0.1 |
| Reverse Primer (100 uM) | 0.1 |
| Sterile Nuclease Free Water | 17.8 |
| SuperScript III RT/Platinum Taq Enzyme Mix | 2.0 |

### Reaction and RNA Sample Loading of Reaction Tubes

1. Mix reaction mixture by vortex and transfer 45.0 uL to each PCR tube corresponding to the number of samples.
2. Add 5.0 uL of purified RNA samples and controls to PCR tubes.
3. Ensure fluids are collected at surface bottom by manual or brief centrifugation (quick spin up to 700 × g).
4. Complete one-step real-time RT-PCR using the cycle as defined below.

TABLE 62
Amplification Cycle

| | | | |
|---|---|---|---|
| | Temperature | | Number |
| Stage | ° C. | Duration | of Cycles |

| | | | | | | |
|---|---|---|---|---|---|---|
| Stage 1 | 50° | C. | 30 | min | 1 | cycle |
| Stage 2 | 95° | C. | 15 | min | 1 | cycle |
| Stage 3 | 95° | C. | 1 | min | 35 | cycles |
| | 50° | C. | 1 | min | | |
| | 72° | C. | 1 | min | | |
| Stage 4 | 72° | C. | 7 | min | 1 | Cycle |
| Stage 5 | 4° | C. | Hold | | 1 | Cycle |

### Analysis

Run 10 μL of reaction mixture on a 1-2% agarose gel capable of discerning an ˜ 300 bp band and record+ if a band is present and—if a band is not present.

TABLE 63
RT-PCR of BVDV infected BK6 and SKB7 Cells.

| | | BVDV-1a | BVDV-1b | BVDV-2 |
|---|---|---|---|---|
| Cell | Uninfected | 5960 | OK1794-1 | C125 |
| Type | Cells | Infection | Infection | Infection |
| BK6 | − | + | + | + |
| SK-B7 | − | + | + | + |

+ indicates the presence of a band on the gel post RT-PCR
− indicates the absence of a band on the gel post RT-PCR

### Conclusions:

BVDV-1a, BVDV-1b and BVDV-2 were capable of growing on the SKB7 cells, albeit at a lower titer than when these viruses are grown on bovine kidney cells (BK6). This was evidenced by and increase in titer from Day 0 to Day 8 of cells infected with BVDV virus and the presence of BVDV RNA in BVDV-infected cells compared to non-infected cells.

### Example 9 Growth of Porcine Epidemic Diarrhea Virus (PEDV) on PERV negative SKB7 cells

### PEDV Virus Growth

### Materials and Reagents

SKB7 cells
PMEM Media (Zoetis)
Fetal bovine serum (Zoetis)
100× GlutaMAX, (Gibco cat #35050-061)
Gentamicin, 10 mg/mL (Gibco part #15710-064)
Bovine rota corona trypsin (Zoetis)
TrypLE select (Gibco 12563-020)
T75 flasks (Corning 430825 or equivalent)
TC20 Automated Cell Counter (BioRad 1450102)
PEDV virus at Log (10) TCID50/ml=5.0 (GenBank Accession NO: KF272920)
Growth Media: PMEM with 5% FBS, 1× GlutaMAX, 20 ug/ml gentamicin.
Infection Media: PMEM, 1× GlutaMAX, 20 ug/ml gentamicin, 2 USP/ml bovine corona trypsin

### Method:

Infect SKB7 cells with PEDV virus:
1. Plate SKB7 cells in 3 × T75 flasks with 2.1×106 cells and 25 ml growth media per flask. One flask will be used to count cells prior to infection, and one will serve as a negative control and the third flask will be used for PEDV infection.
2. Incubate at 37 C, 5% CO₂ for 4 days.
3. After four days, wash one of the SKB7 T75 flasks with 10 ml TrypLE select 1 time and add back 10 ml of TrypLE select for cell disassociation.
4. Incubate flask for 10 minutes at 37C, then add back 10 ml growth media and count cells using TC20 Automated Cell counter.
5. SKB7 Cell count was 7.15e5/mL×30 mL=2.14e7 total cells To achieve MOI=. 003 *2.14 e7 total cells =6.4e4 virus particles PEDV Virus is at =5.85 Log10 TCID50/mL =6.4e4 virus particles/707946 particles/mL=90.4 ul
5. Remove media from remaining 2 × T75 flasks with SKB7 cells and gently rinse wells with twice with infection media.
6. Add back 20 ml infection media and incubate for 1 hour
7. Dilute 90.4 ul PEDV virus into 10 mL PEDV infection media and add to one SKB7 flask. Add 10 ml infection media without PEDV to a second SKB7 flask with infection media to be used as a negative control. Incubate both flasks at 37C, 5% CO₂
8. Take a 0.8 ml sample for PEDV RT-qPCR from day 0 to day 4.
9. After 4 days incubation, freeze flasks at −80C overnight.

### Titration of SKB7 Flasks

### Materials and Reagents

Frozen T75 flasks from PEDV growth experiment
50 ml conical tubes (Corning 4558 or equivalent)
Centrifuge (Thermo Scientific, Sorvall Legend XTR or equivalent)
96 well plates (Corning Costar Cat #3585)
Assay Blocks, 2 mL (Costar Cat #3960)
TC20 Automated Cell Counter (BioRad 1450102)
PMEM Media (Zoetis)
Fetal bovine serum (Zoetis)
100× GlutaMAX, (Gibco cat #35050061)
Gentamicin, 10 mg/mL (Gibco part #15710064)
Bovine rota corona trypsin (Zoetis)
PEDV positive assay control (GenBank Accession NO KF272920)
Anti PEDV antibody (Zoetis)
AlexaFluor 488 AffiniPure Goat Anti-Mouse IgG (H+L) (Jackson Immuno Research cat #115-545-003) diluted 1:250 in PBS
DPBS (Gibco 14190-144)
Global Vero cells (Zoetis)
Biomek 4000 or equivalent
Matrix multichannel pipettor or equivalent
Incubator at 37° C. with 5% CO₂
Fluorescent microscope
General lab supplies (pipettes, tips, etc)
80% Acetone in PBS
Growth media: PMEM-W (Zoetis) with 5% FBS (Zoetis), 1× Glutamax (Invitrogen), 10 mg/ml gentamicin (Invitrogen)
Infection media: PMEM-W (Pfizer Lincoln 45023162 or equivalent) with (Zoetis GMS), 1×Glutamax (Invitrogen), 10 mg/mL gentamicin (Invitrogen), 2 USP/mL Bovine Corona trypsin

### Method

Titration of SKB7 Flasks:
1. Thaw the frozen T75 flasks at room temperature.
2. Remove the contents of the T75 flasks and transfer to 50 ml conical tubes.
3. Centrifuge the tubes at 3000 rpm for 10 minutes to remove cell debris.
4. Use the supernatant for titration. Titrate the samples to determine PEDV TCID50 values.
5. Seed 96-well plates with 30,000 Global Vero cells per well, 0.1 ml media per well. Media is PMEM-W, 5% FBS, 1× Glutamax, 20 μg/mL gentamicin. Incubate cells 4 days at 37C and 5% CO₂.
6. After 4 days, remove media from cells and wash 1× with Infection Media, 100 uL per well.
7. Remove media and add 100 uL Infection Media per well. Incubate 45-60 min at 37° C.
8. Fill assay block (Costar or similar) with 0.9 ml infection media (same formulation as above) using a Biomek NX or Matrix pipettor.
9. Add 0.1 ml of each virus sample to column 1 of the deep-well cube.
10. Perform 1:10 serial dilutions (0.1 ml into 0.9 ml) as needed, using a Biomek 3000 or Matrix pipettor. Change tips in between dilutions.
11. Add 0.1 ml of each PEDV virus dilution to each cell plate in 8 replicates (down the plate).
12. Incubate the cell plates at 37° C., 5% CO₂ for 4 days.
13. Discard supernatant in Biohazard bag. Fix with ice-cold 80% acetone and let stand for 15 minutes at room temperature.
14. Discard fixative in Biohazard bag. Allow the plates to air dry for 15 minutes.
15. Add 100 uL MAb PEDV diluted 1:1000 in PBS, to each well. Incubate at 37° C. for 1 hour.
16. Wash plates 3-5 times by submerging them in distilled water. Dump final wash and blot on a paper towel.
17. Add 100 uL Alexafluor 488 goat anti-mouse IgG (H+L) or AlexaFluor 488 goat anti-swine diluted 1:250 in PBS to each well. Incubate at 37° C. for 1 hour.
18. Wash plates three-five times by submerging them in distilled water. Dump final wash and blot on a paper towel.
19. Score the plates using a fluorescent microscope with an ultra-violet light source.
20. Calculate titers using the Spearman-Karber method.
   RNA purification and PEDV RT qPCR Assay

### Materials and Reagents

IndiSpin Pathogen NA Extraction Kit (Indical SP54104)

PEDV N gene-F
(SEQ ID NO: 31)
5′-GAATTCCCAAGGGCGAAAAT-3′, 100 μM stock
PEDV N gene-R
(SEQ ID NO: 32)
5′-TTTTCGACAAATTCCGCATCT-3′, 100 μM stock
PEDV Probe 6FAM
(SEQ ID NO: 33)
5′-CGTAGCAGCTTGCTTCGGACCCA 3′ TAMRA,, 100 μM stock

Path ID Multiplex One-Step RT-PCR kit (Ambion/Life Technolgies 4428407)
Standard Curve PEDV N gene DNA (stored frozen at −20° C.)
Nuclease-free water (Ambion AM9937 or equivalent)
Bio-Rad CFX384 with CFX manager software
PCR plates (Bio-Rad HSP-9601)
96-well U shape microplate (Greiner, 650101)
Microseal B film (Bio-Rad MSB1001)
Quarter Modular Reservoir (Beckman Coulter 372788)
General Lab Supplies

### Method

### Preparation of Assay Master Mix

1. Prepare master mix without RNA sample, yielding a total final volume of 10 ul per reaction. Each reaction should contain the following components.
2. Adjust volume of each component according to number of samples to be tested in the assay.

TABLE 64
Components for PEDV RT-qPCR Reaction

| Component | Volume | |
|---|---|---|
| Multiplex RT-PCR Buffer | 7.5 | μl |
| Multiplex Enzyme Mix | 1.50 | μl |
| PEDV F (Forward Primer@ 100 uM) | 0.045 | μl |
| PEDV R (Reverse Primer @ 100 uM) | 0.045 | μl |
| PEDV Probe (FAM) @ 100 uM | 0.038 | μl |
| Nuclease-Free Water | 0.872 | μl |

### RTqPCR:

1. In one side of a quarter-module reservoir divided by length, prepare RT-qPCR reagents according to table 64 above. Multiply each component above by the number of samples and standard curve replicates needed for each assay.
2. In the other side of the quarter-module reservoir, add 3 mL of nuclease-free water.
3. Add 50-100 uL of each RNA sample to a Greiner clear U-bottom 96-well plate. Add sample 1 to well A1, sample 2 to well B1, etc. Samples are arranged in columns, not rows.
4. Thaw one vial of PEDV standard. Add 50-100 ul to well A1 of a Greiner 96-well plate. Prepare a standard curve by performing serial dilutions of the PEDV DNA in nuclease-free water from 1e6 DNA copies/5 ul through 1e0 DNA copies/5 ul. Add 5 ul each standard curve dilution, “no template controls” (NTC)
5. Use a Biomek method for 384 well PCR plate layout to load master mix, samples, and standard curve components onto a 384 well plate.
6. Cover the assay plate with an adhesive plate cover and centrifuge briefly (2000 RPM for ˜20 seconds) to collect fluids to the bottoms of the wells.
7. Load the plate into the CFX384 Thermocycler and use thermocycling parameters in Table 65 below:

TABLE 65
PEDV RT-qPCR Thermocycle Reaction

| Temp | Time | |
|---|---|---|
| 50 | 10:00 | |
| 95 | 5:00 | |
| 95 | 0:15 | X |
| 55 | 0:30 | 40 |

8. The test samples are compared to the standard curve to determine the copy number present in each sample. Data can be recorded as Ct value or copy number/5 ul sample. The Ct cut-off value for this assay is 35 cycles, any value above 35 cycles is considered negative.

### Results:

Table 66 below represents the TCID50 for SKB7 cells at Day 4, both uninfected and infected with PEDV.

TABLE 66
PEDV TCID50 Results

| | Log₁₀ TCID₅₀ / mL |
|---|---|
| Sample | Day 4 |
| SKB7 Cells | ≤1.50* |
| SKB7 Cells + PEDV (0.01 MOI) | 4.44 |
| Assay Control | 4.13 |

*= Limit of detection of assay

The log₁₀ TCID₅₀/mL of the PEDV infected flask was 4.44 compared to the uninfected flask that had an titer below the limit of detection of 1.50 log₁₀ TCID₅₀/mL.

Copy number of PEDV inoculated flask increased from 0 to 4 day after infection, as measured by RT qPCR (Table 67 below). The flask without PEDV remained at 0 copies per ml. These results indicate that the PEDV virus is growing in the SKB7 cell line.

TABLE 67
PEDV RT-qPCR results

| | |
|---|---|
| | PEDV Copy |
| Day Post | Number/ 5 ul |

| Infection | PEDV | Uninfected |
|---|---|---|
| Day 0 | 1000 | 0 |
| Day 1 | 1520 | 0 |
| Day 2 | 1937 | 0 |
| Day 3 | 2627 | 0 |
| Day 4 | 13367 | 0 |

The results in Table 68 below confirm the PEDV RT-qPCR assay ran correctly since the positive control (standards) gave the expected copy number and Cq values and the non-template control (NTC) was not recorded (N/A) in the assay. The N/A designation indicates no PEDV nucleic acid was present in the sample.

TABLE 68
RT-qPCR PEDV Dilution Standard

| Sample | Target copies | Cq |
|---|---|---|
| Std 01 | 1,000,000 | 13.44 |
| Std 02 | 100,000 | 16.18 |
| Std 03 | 10,000 | 19.33 |
| Std 04 | 1,000 | 22.16 |
| Std 05 | 100 | 25.31 |
| Std 06 | 10 | 28.23 |
| Std 07 | 1 | 33.87 |
| NTC | 0 | N/A |

### Conclusions:

The SKB7 cell line can support the growth of PEDV based on the titration results after 4 days of infection and the increase in copy number of PEDV from Day 0 to Day 4 in the RT-qPCR reaction. The PEDV RT-qPCR reaction would have shown a consistent or decreased PEDV copy number if the PEDV virus was not replicating in the SKB7 cell line.

What is claimed is:

1. A method of preparing a vaccine, comprising:
infecting gene-edited porcine endogenous retrovirus (PERV) negative swine cells with a microorganism which expresses at least one protein antigen capable of inducing protective immunity in an animal against an infectious agent;
culturing the infected cells in culture medium to propagate the microorganism; and
harvesting the propagated microorganism from the culture medium to obtain a fraction comprising a PERV free antigen for use in immunizing an animal against the infectious agent.

2. The method of claim 1, wherein the PERV free antigen comprised in the fraction is the propagated microorganism or is derived therefrom.

3. The method of claim 1, further comprising isolating the PERV free antigen from the propagated microorganism.

4. The method of claim 1, wherein the PERV free antigen is a live antigen.

5. The method of claim 4, further comprising lyophilizing the PERV free live antigen.

6. The method of claim 4, further comprising inactivating the PERV free live antigen with a chemical inactivant.

7. The method of claim 6, further comprising removing the chemical inactivant.

8. The method of claim 6, further comprising combining the inactivated PERV free antigen with an adjuvant.

9. The method of claim 1, further comprising separating the PERV free antigen from cellular material.

10. The method of claim 1, wherein the microorganism used to infect the PERV negative swine cells is contained in a cell lysate.

11. The method of claim 1, further comprising employing culture medium containing the infected PERV negative swine cells as seed material for serial passages to produce a further amount of the PERV free antigen.

12. The method of claim 1, further comprising concentrating the PERV free antigen.

13. The method of claim 12, wherein the PERV free antigen is concentrated by ultrafiltration.

14. The method of claim 12, further comprising washing the concentrated PERV free antigen with a balanced salt solution to reduce serum proteins.

15. The method of claim 1, further comprising combining the PERV free antigen with an additional antigen.

16. The method of claim 1, further comprising combining the PERV free antigen with a pharmaceutically acceptable carrier.

17. The method of claim 1, wherein the microorganism used to infect the PERV negative swine cells is selected from the group consisting of a porcine circovirus (PCV), porcine reproductive and respiratory syndrome virus (PRRSV), Porcine parvovirus (PPV), Influenza A Virus of Swine (IAV-S), Porcine epidemic diarrhea virus (PEDV), Swine delta coronavirus (SDCoV), Lawsonia intracellularis, Salmonella, Bovine Viral Diarrhea Virus-Type 1 (BVDV-1) and Bovine Viral Diarrhea Virus-Type 1 (BVDV-2).

18. The method of claim 17, wherein the microorganism used to infect the PERV negative swine cells is a porcine circovirus.

19. The method of claim 18 wherein the porcine circovirus is a wild-type virus.

20. The method of claim 18, wherein the porcine circovirus is a chimeric whole virus.

21. The method of claim 20, wherein the chimeric whole virus is a recombinant PCV1-2 virus comprising the ORF1 replicase of PCV1 and the ORF2 of a pathogenic PCV2 genotype.

22. The method of claim 21, wherein the ORF2 is from a PCV2 genotype selected from the group consisting of PCV2 type 2a, PCV2 type 2b, PCV2 type 2d, and combinations thereof.

23. The method of claim 22, wherein the ORF2 is from a PCV2d genotype.

24. The method of claim 1, wherein the PERV negative swine cells infected with the microorganism are PERV negative PK-15 cells.

25. The method of claim 24, wherein the PERV negative PK-15 cells are infected with a recombinant PCV1-2 virus comprising the ORF1 replicase of PCV1 and the ORF2 of a PCV2 genotype.

26. The method of claim 25, wherein the ORF2 is from a PCV2 genotype selected from the group consisting of PCV2 type 2a, PCV2 type 2b, PCV2 type 2d, and combinations thereof.

27. The method of claim 1, wherein the PERV negative swine cells infected with the microorganism are engineered to express porcine CD163.

28. The method of claim 27, wherein the PERV negative swine cells engineered to express porcine CD163 are infected with PRRS virus.

29. The method of claim 1, wherein the PERV negative swine cells comprise PERV sequences disrupted at genetic locations within the PERV pol gene, wherein one of said genetic locations is within the catalytic region of the PERV pol gene and another of said genetic locations is upstream of the catalytic region of the PERV pol gene.

30. A porcine endogenous retrovirus (PERV) negative swine cell line, wherein the cell line comprises PERV sequences disrupted at genetic locations within the PERV pol gene, wherein one of said genetic locations is within the catalytic region of the PERV pol gene and another of said genetic locations is upstream of the catalytic region of the PERV pol gene.

31. The PERV negative swine cell line of claim 30, wherein the swine cell line is a porcine kidney (PK) cell line.

32. The PERV negative swine cell line of claim 30, wherein the cell line is produced by a method comprising:
(a) introducing into parent swine cells (i) two guide ribonucleic acids (gRNAs) that target the PERV pol gene, wherein one of said gRNAs targets the catalytic region of the PERV pol gene and the other of said gRNAs targets a region upstream of the catalytic region of the PERV pol gene; and (ii) a nucleic acid sequence that encodes a Cas protein; and
(b) culturing the cells under suitable conditions in which the Cas protein is expressed and the gRNAs recruit the Cas protein to the site of targeted PERV pol gene;
(c) allowing the Cas protein to create double-stranded breaks in multiple copies of the targeted PERV pol gene, thereby inactivating expression of said copies.

33. The PERV negative swine cell line of claim 32, wherein the Cas protein in a Cas9 protein.

34. The PERV negative swine cell line of claim 32, wherein all functional copies of the targeted PERV pol gene are inactivated.

35. The PERV negative swine cell line of claim 32, wherein the gRNA that targets the catalytic region of the PERV pol gene comprises the sequence:

| (SEQ ID NO: 6) | |
|---|---|
| | 5′ TACTGGAGGAGGGTCACCTG 3′. |

36. The PERV negative swine cell line of claim 32, wherein the gRNA that targets a region upstream of the catalytic region of the PERV pol gene comprises the sequence:

| (SEQ ID NO: 4) | |
|---|---|
| | 5′ ACCAGTACAGGACTTGAG 3′. |

37. A PERV free vaccine for use in protecting a pig against a swine pathogen, wherein the vaccine comprises an immunogenic fraction derived from one or more microorganisms capable of infecting the PERV-negative swine cell line of claim 30.

38. The PERV free vaccine of claim 37 wherein the one or more microorganisms are selected from the group consisting of viruses, intracellular bacteria, and combinations thereof.

39. The PERV free vaccine of claim 37, wherein the one or more microorganisms is an intracellular parasite.

40. The PERV free vaccine of claim 37, wherein the pig is a PERV free pig.

41. PERV free cultures of microorganisms, wherein said PERV free cultures are propagated on the PERV free cell line of claim 30.

42. The PERV free cultures of microorganisms of claim 41, wherein the microorganisms are selected from the group consisting of viruses, bacteria, and combinations thereof.

43. An allelic variant of the porcine endogenous retrovirus (PERV) pol gene wherein the allelic variant results from a disruption in the PERV pol gene at about nucleotide 437 to about nucleotide 971 of the PERV pol consensus gene represented of SEQ ID NO: 1, and wherein said allelic variant is capable of conferring loss of PERV function upon a swine cell line.

44. The allelic variant of claim 43, wherein the allelic variant results from a disruption in the PERV pol gene at about nucleotides 537 to about 853 of the PERV pol consensus gene represented by SEQ ID NO: 1.

45. A method of preparing a vaccine, comprising:
transfecting gene-edited PERV negative swine cells with DNA or RNA carrying sequence encoding the viral proteins required for viral packaging, assembly, and recovery of a virus;
culturing the transfected cells in culture medium to propagate the virus; and
recovering the propagated virus from the culture medium to obtain a fraction comprising a PERV free antigen for use in immunizing an animal against an infectious agent.

46. The method of claim 45, wherein the PERV free antigen comprised in the fraction is the virus or is derived therefrom.

47. The method of claim 45, further comprising isolating the PERV free antigen from the virus.

48. The method of claim 45, wherein the PERV free antigen is a live antigen.

49. The method of claim 48, further comprising lyophilizing the PERV free live antigen.

50. The method of claim 48, further comprising inactivating the PERV free live antigen with a chemical inactivant.

51. The method of claim 50, further comprising combining the inactivated PERV free antigen with an adjuvant.

52. The method of claim 45, further comprising combining the PERV free antigen with an additional antigen.

53. The method of claim 45, further comprising combining the PERV free antigen with a pharmaceutically acceptable carrier.

54. The method of claim 45, wherein the PERV negative cells are transfected with DNA or RNA encoding the viral proteins required for viral packaging, assembly, and recovery of an antigenic recombinant PCV1-2 virus comprising the ORF1 replicase of PCV1 and the ORF2 of a PCV2 genotype.

55. The method of claim 54, wherein the ORF2 is from a PCV2 genotype selected from the group consisting of PCV2 type 2a, PCV2 type 2b, PCV2 type 2d, and combinations thereof.

56. A vaccine composition comprising an antigenic virus made by the method claim 45.


SEQUENCE LISTING (33 sequences)

SEQ ID NO: 1 - DNA, 3441 bp, Sus scrofa
  source 1..3441: mol_type = other DNA; note = consensus PERV Polymerase gene sequence
atgggtgccacagggcaacggcagtatccatggactacccgaagaaccgttgacttgggagtgggacgggtaacccactcgtttctggtcatccctgagtgcccagtaccccttctaggtagagacttactgaccaagatgggagctcaaatttcttttgaacaaggaagaccagaagtgtctgcgaataacaaacccatcactgtgttgaccctccaattagatgatgaatatcgactatattctccccaagtaaagcctgatcaagatatacagtcctggttggagcagtttccccaagcctgggcagaaaccgcagggatgggtttggcaaagcaagttcccccacaggttattcaactgaaggccagtgctacaccagtatcagtcagacagtaccccttgagtagagaggctcgagaaggaatttggccgcatgttcaaagattaatccaacagggcatcctagttcctgtccaatccccttggaatactcccctgctaccggttaggaagcctgggaccaatgattatcgaccagtacaggacttgagagaggtcaataaaagggtgcaggacatacacccaacggtcccgaacccttataacctcttgagcgccctcccgcctgaacggaactggtacacagtattggacttaaaagatgccttcttctgcctgagattacaccccactagccaaccactttttgccttcgaatggagagatccaggtacgggaagaaccgggcagctcacctggacccgactgccccaagggttcaagaactccccgaccatctttgacgaagccctacacagggacctggccaacttcaggatccaacaccctcaggtgaccctcctccagtacgtggatgacctgcttctggcgggagccaccaaacaggactgcttagaaggtacgaaggcactactgctggaattgtctgacctaggctacagagcctctgctaagaaggcccagatttgcaggagagaggtaacatacttggggtacagtttgcggggcgggcagcgatggctgacggaggcacggaagaaaactgtagtccagataccggccccaaccacagccaaacaagtgagagagtttttggggacagctggattttgcagactgtggatcccggggtttgcgaccttagcagccccactctacccgctaaccaaagaaaaaggggaattctcctgggctcctgagcaccagaaggcatttgatgctatcaaaaaggccctgctgagcgcacctgctctggccctccctgacgtaactaaaccctttaccctttatgtggatgagcgtaagggagtagcccgaggagttttaacccaaaccctaggaccatggaggagacctgttgcctacctgtcaaagaagcttgatcctgtagccagtggttggcccgtatgcctgaaggctatcgcagctgtggccatactggtcaaggacgctgacaaattgactttgggacagaatataactgtaatagccccccatgcattggagaacatcgttcggcagcccccagaccgatggatgaccaacgcccgcatgacccactatcaaagcctgcttctcacagagagggtcacgttcgctccaccagccgctttcaaccctgccacttttctgcctgaagagactgatgaaccagtgactcatgattgccatcaactattgattgaggagactggggtccgcaaggaccttacagacataccgctgactggagaagtgctaacctggttcactgacggaagcagctatgtggtggaaggtaagaggatggctggggcggcggtggtggacgggacccgcacgatctgggccagcagcctgccagaaggaacttcagcgcaaaaggctgagctcatggccctcacacaagctttgcggctggccgaagggaaatccataaacatttatacggacagcaggtatgcctttgcgactgcacacgtacacggggccatctataaacaaagggggttgcttacctcagcagggagggaaataaagaacaaagaggaaattctaagcctattagaagccttacatttgccaaaaaggctagctattatacactgtcctggacatcagaaagccaaagatctcatatctagagggaaccagatggctgaccgggttgccaagcaggcagcccaggctgttaaccttctgcctataatagaaacgcccaaagccccagaacccagacgacagtacaccctagaagactggcaagagataaaaaagatagaccagttctctgagactccggaggggacctgctatacctcatatgggaaggaaatcctgccccacaaagaagggttagaatatgtccaacagatacatcgtctcacccacctaggaactaaacacctgcagcagttggtcagaacatccccttatcatgttctgaggctaccaggagtggctgactcggtggtcaaacattgtgtgccctgccagctggttaatgctaatccttccagaatacctccaggaaagagactaaggggaagccacccaggcgctcactgggaagtggacttcactgaggtaaagccggctaaatacggaaacaaatatctattggtttttgtagacaccttttcaggatgggtagaggcttatcctactaagaaagagacttcaaccgtggtggctaagaaaatactggaggaaatttttccaagatttggaatacctaaagtaatcgggtcagacaatggtccagcttttgttgcccaggtaagtcagggactggccaagatattggggattgattggaaattacattgtgcatacagaccccaaagctcaggacaggtagagaggatgaatagaaccattaaagagacccttaccaaattgaccacagagactggcattaatgattggatagctctcctgccctttgtgctctttagggttaggaacacccctggacagtttgggctgaccccctatgaattgctctacgggggaccccccccgttggtagaaattgcttctgtacatagtgctgatgtgctgctttcccagcctttgttctctaggctcaaggcgctcgagtgggtgaggcaacgagcgtggaagcagctccgggaggcctactcaggagaaggagacttgcaagttccacatcgcttccaagtgggagattcagtctatgttagacgccaccgtgcaggaaacctcgagactcggtggaagggcccttatctcgtacttttgaccacaccaacggctgtgaaagtcgaaggaatctccacctggatccatgcatcccacgttaagctggcgccacctcccgactcggggtggagagccgaaaagactgagaatccccttaagcttcgcctccatcgcctggttccttactctaacaataactccccaggccagtag

SEQ ID NO: 2 - RNA, 20 nt, synthetic construct
  source 1..20: mol_type = other RNA; note = sequence for gRNA 1848, originally disclosed by Yang et al
ggtcatccacgtactggagg

SEQ ID NO: 3 - RNA, 20 nt, synthetic construct
  source 1..20: mol_type = other RNA; note = sequence for gRNA 1849, originally disclosed by Yang et al
ggtgaccctcctccagtacg

SEQ ID NO: 4 - RNA, 20 nt, synthetic construct
  source 1..20: mol_type = other RNA; note = sequence for gRNA 1844
accagtacaggacttgagag

SEQ ID NO: 5 - RNA, 20 nt, synthetic construct
  source 1..20: mol_type = other RNA; note = sequence for gRNA 1845
cttgaacccttggggcagtc

SEQ ID NO: 6 - RNA, 20 nt, synthetic construct
  source 1..20: mol_type = other RNA; note = sequence for gRNA 1847
tactggaggagggtcacctg

SEQ ID NO: 7 - RNA, 20 nt, synthetic construct
  source 1..20: mol_type = other RNA; note = sequence for gRNA 1850
tctggcgggagccaccaaac

SEQ ID NO: 8 - DNA, 52 bp, synthetic construct
  source 1..52: mol_type = other DNA; note = PERV guide DNA sequence Guide RNA 1844
tgattatcgaccagtacaggacttgagagaggtcaataaaagggtgcaggac

SEQ ID NO: 9 - DNA, 52 bp, synthetic construct
  source 1..52: mol_type = other DNA; note = PERV guide DNA sequence Guide RNA 1847
caacaccctcaggtgaccctcctccagtacgtggatgacctgcttctggcgg

SEQ ID NO: 10 - DNA, 18 bp, synthetic construct
  source 1..18: mol_type = other DNA; note = forward primer termed P12963
gggagtgggacgggtaac

SEQ ID NO: 11 - DNA, 21 bp, synthetic construct
  source 1..21: mol_type = other DNA; note = reverse primer termed P12964
gtcaaagatggtcggggagtt

SEQ ID NO: 12 - DNA, 20 bp, synthetic construct
  source 1..20: mol_type = other DNA; note = forward sequencing primer termed P12677
gtgcccagtaccccttctag

SEQ ID NO: 13 - DNA, 20 bp, synthetic construct
  source 1..20: mol_type = other DNA; note = forward primer termed P12981
gatgccttcttctgcctgag

SEQ ID NO: 14 - DNA, 20 bp, synthetic construct
  source 1..20: mol_type = other DNA; note = reverse primer termed P12304
aaaactcctcgggctactcc

SEQ ID NO: 15 - DNA, 20 bp, synthetic construct
  source 1..20: mol_type = other DNA; note = reverse sequencing primer termed P12303
ggctactcccttacgctcat

SEQ ID NO: 16 - DNA, 1762 bp, synthetic construct
  source 1..1762: mol_type = other DNA; note = nucleotide sequence of a chimeric PCV1-2d virus described herein
accagcgcacttcggcagcggcagcacctcggcagcgtcagtgaaaatgccaagcaggaaaagcggcccgcaaccccataagaggtgggtgttcacccttaataatccttccgaggaggagaaaaacaaaatacgggagcttccaatctccctttttgattattttgtttgcggagaggaaggtttggaagagggtagaactcctcacctccaggggtttgcgaattttgctaagaagcagacttttaacaaggtgaagtggtattttggtgcccgctgccacatcgagaaagcgaaaggaaccgaccagcagaataaagaatactgcagtaaagaaggccacatacttatcgagtgtggagctccgcggaaccaggggaagcgcagcgacctgtctactgctgtgagtacccttttggagacggggtctttggtgactgtagccgagcagttccctgtaacgtatgtgagaaatttccgcgggctggctgaacttttgaaagtgagcgggaagatgcagcagcgtgattggaagacagctgtacacgtcatagtgggcccgcccggttgtgggaagagccagtgggcccgtaattttgctgagcctagcgacacctactggaagcctagtagaaataagtggtgggatggatatcatggagaagaagttgttgttttggatgatttttatggctggttaccttgggatgatctactgagactgtgtgaccggtatccattgactgtagagactaaagggggtactgttccttttttggcccgcagtattttgattaccagcaatcaggccccccaggaatggtactcctcaactgctgtcccagctgtagaagctctctatcggaggattactactttgcaattttggaagactgctggagaacaatccacggaggtacccgaaggccgatttgaagcagtggacccaccctgtgcccttttcccatataaaataaattactgagtcttttttgttatcacatcgtaatggtttttatttttacttagggttaagtggggggtctttaagattaaattctctgaattgtacatacatggttatacggatattgtagtcctggtcgtatatactgttttcgaacgcagtgccgaggcctacatggtctacatttccagtagtttgtagtctcagccagagttgatttcttttgttattgggttggaagtaatcgattgtcctatcaaggacaggtttcggggtaaagtaccgggagtggtaggagaagggctgggttatggtatggcgggaggagtagtttacataggggtcataggttagggcattggcctttgttacaaagttatcatctagaataacagcagtggagcccactcccctgtcaccctgggtgattggggagcagggccagaattcaaccttaaccttccttattctgtagtattcaaagggcacagtgagggggtttgagccccctcctgggggaagaaaatcattaatattaaatctcatcatgtccacattccaggagggcgttctgactgtggttttcttgacagtataaccgatggtgcgggagaggcgggtgttgaagatgccatttttccttctccagcggtaacggtggcgggggtggacgagccaggggcggcggcggaggatctggccaagatggctgcgggggcggtgtcttcgtctgcggaaacgcctccttggatacgtcatcctataaaagtgaaagaagtgcgctgctgtagtatt

SEQ ID NO: 17 - DNA, 2088 bp, synthetic construct
  source 1..2088: mol_type = other DNA; note = nucleotide sequence of a chimeric PCV1-2d virus
ggccaaaaaggccttatggctggttaccttgggatgatctactgagactgtgtgaccggtatccattgactgtagagactaaagggggtactgttccttttttggcccgcagtattttgattaccagcaatcaggccccccaggaatggtactcctcaactgctgtcccagctgtagaagctctctatcggaggattactactttgcaattttggaagactgctggagaacaatccacggaggtacccgaaggccgatttgaagcagtggacccaccctgtgcccttttcccatataaaataaattactgagtcttttttgttatcacatcgtaatggtttttatttttacttagggttaagtggggggtctttaagattaaattctctgaattgtacatacatggttatacggatattgtagtcctggtcgtatatactgttttcgaacgcagtgccgaggcctacatggtctacatttccagtagtttgtagtctcagccagagttgatttcttttgttattgggttggaagtaatcgattgtcctatcaaggacaggtttcggggtaaagtaccgggagtggtaggagaagggctgggttatggtatggcgggaggagtagtttacataggggtcataggttagggcattggcctttgttacaaagttatcatctagaataacagcagtggagcccactcccctgtcaccctgggtgattggggagcagggccagaattcaaccttaaccttccttattctgtagtattcaaagggcacagtgagggggtttgagccccctcctgggggaagaaaatcattaatattaaatctcatcatgtccacattccaggagggcgttctgactgtggttttcttgacagtataaccgatggtgcgggagaggcgggtgttgaagatgccatttttccttctccagcggtaacggtggcgggggtggacgagccaggggcggcggcggaggatctggccaagatggctgcgggggcggtgtcttcgtctgcggaaacgcctccttggatacgtcatcctataaaagtgaaagaagtgcgctgctgtagtattaccagcgcacttcggcagcggcagcacctcggcagcgtcagtgaaaatgccaagcaggaaaagcggcccgcaaccccataagaggtgggtgttcacccttaataatccttccgaggaggagaaaaacaaaatacgggagcttccaatctccctttttgattattttgtttgcggagaggaaggtttggaagagggtagaactcctcacctccaggggtttgcgaattttgctaagaagcagacttttaacaaggtgaagtggtattttggtgcccgctgccacatcgagaaagcgaaaggaaccgaccagcagaataaagaatactgcagtaaagaaggccacatacttatcgagtgtggagctccgcggaaccaggggaagcgcagcgacctgtctactgctgtgagtacccttttggagacggggtctttggtgactgtagccgagcagttccctgtaacgtatgtgagaaatttccgcgggctggctgaacttttgaaagtgagcgggaagatgcagcagcgtgattggaagacagctgtacacgtcatagtgggcccgcccggttgtgggaagagccagtgggcccgtaattttgctgagcctagcgacacctactggaagcctagtagaaataagtggtgggatggatatcatggagaagaagttgttgttttggatgatttttatggctggttaccttgggatgatctactgagactgtgtgaccggtatccattgactgtagagactaaagggggtactgttccttttttggcccgcagtattttgattaccagcaatcaggccccccaggaatggtactcctcaactgctgtcccagctgtagaagctctctatcggaggattactactttgcaattttggaagactgctggagaacaatccacggaggtacccgaaggccgatttgaagcagtggacccaccctgtgcccttttcccatataaaataaattactgagtggccaaaaaggcc

SEQ ID NO: 18 - DNA, 939 bp, synthetic construct
  source 1..939: mol_type = other DNA; note = nucleotide sequence of the PCV1 ORF1
atgccaagcaggaaaagcggcccgcaaccccataagaggtgggtgttcacccttaataatccttccgaggaggagaaaaacaaaatacgggagcttccaatctccctttttgattattttgtttgcggagaggaaggtttggaagagggtagaactcctcacctccaggggtttgcgaattttgctaagaagcagacttttaacaaggtgaagtggtattttggtgcccgctgccacatcgagaaagcgaaaggaaccgaccagcagaataaagaatactgcagtaaagaaggccacatacttatcgagtgtggagctccgcggaaccaggggaagcgcagcgacctgtctactgctgtgagtacccttttggagacggggtctttggtgactgtagccgagcagttccctgtaacgtatgtgagaaatttccgcgggctggctgaacttttgaaagtgagcgggaagatgcagcagcgtgattggaagacagctgtacacgtcatagtgggcccgcccggttgtgggaagagccagtgggcccgtaattttgctgagcctagcgacacctactggaagcctagtagaaataagtggtgggatggatatcatggagaagaagttgttgttttggatgatttttatggctggttaccttgggatgatctactgagactgtgtgaccggtatccattgactgtagagactaaagggggtactgttccttttttggcccgcagtattttgattaccagcaatcaggccccccaggaatggtactcctcaactgctgtcccagctgtagaagctctctatcggaggattactactttgcaattttggaagactgctggagaacaatccacggaggtacccgaaggccgatttgaagcagtggacccaccctgtgcccttttcccatataaaataaattactga

SEQ ID NO: 19 - DNA, 705 bp, synthetic construct
  source 1..705: mol_type = other DNA; note = nucleotide sequence of the PCV2d ORF2
atgacgtatccwaggaggcgtttccgcagacgaagacaccgcccccgcagccatcttggccagatcctccgccgccgcccctggctcgtccacccccgccaccgttaccgctggagaaggaaaaatggcatcttcaacacccgcctctcccgcaccatcggttatactgtcaagaaaaccacagtcagaacgccctcctggaatgtggacatgatgagatttaatattaatgattttcttcccccaggagggggctcaaaccccctcactgtgccctttgaatactacagaataaggaaggttaaggttgaattctggccctgctccccaatcacccagggtgacaggggagtgggctccactgctgttattctagatgataactttgtaacaaaggccaatgccctaacctatgacccctatgtaaactactcctcccgccataccataacccagcccttctcctaccactcccggtactttaccccgaaacctgtccttgataggacaatcgattacttccaacccaataacaaaagaaatcaactctggctgagactacaaactactggaaatgtagaccatgtaggcctcggcactgcgttcgaaaacagtatatacgaccaggactacaatatccgtataaccatgtatgtacaattcagagaatttaatcttaaagaccccccacttaaccctaagtga

SEQ ID NO: 20 - protein, 234 aa, synthetic construct
  source 1..234: note = amino acid sequence of the full-length PCV2d ORF2
MTYPRRRFRRRRHRPRSHLGQILRRRPWLVHPRHRYRWRRKNGIFNTRLSRTIGYTVKKTTVRTPSWNVDMMRFNINDFLPPGGGSNPLTVPFEYYRIRKVKVEFWPCSPITQGDRGVGSTAVILDDNFVTKANALTYDPYVNYSSRHTITQPFSYHSRYFTPKPVLDRTIDYFQPNNKRNQLWLRLQTTGNVDHVGLGTAFENSIYDQDYNIRITMYVQFREFNLKDPPLNPK

SEQ ID NO: 21 - DNA, 2579 bp, synthetic construct
  source 1..2579: mol_type = other DNA; note = sequence of the plasmid pUC57-Kan
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcgagctcggtacctcgcgaatacatctagatatcggatcccgggcccgtcgactgcagaggcctgcatgcaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 22 - DNA, 1767 bp, Sus scrofa
  source 1..1767: mol_type = genomic DNA; note = sequence of a PCV2d
gtcttttttatcacttcgtaatggtttttattattcacttagggttaagtggggggtctttaagattaaattctctgaattgtacatacatggttatacggatattgtagtcctggtcgtatatactgttttcgaacgcagtgccgaggcctacatggtctacatttccagtagtttgtagtctcagccagagttgatttcttttgttattgggttggaagtaatcgattgtcctatcaaggacaggtttcggggtaaagtaccgggagtggtaggagaagggctgggttatggtatggcgggaggagtagtttacataggggtcataggttagggcattggcctttgttacaaagttatcatctagaataacagcagtggagcccactcccctgtcaccctgggtgattggggagcagggccagaattcaaccttaaccttccttattctgtagtattcaaagggcacagtgagggggtttgagccccctcctgggggaagaaaatcattaatattaaatctcatcatgtccacattccaggagggcgttctgactgtggttttcttgacagtataaccgatggtgcgggagaggcgggtgttgaagatgccatttttccttctccagcggtaacggtggcgggggtggacgagccaggggcggcggcggaggatctggccaagatggctgcgggggcggtgtcttcgtctgcggaaacgcctccttggatacgtcatcgctgaaaacgaaagaagtgcgctgtaagtattaccagcgcacttcggcagcggcagcacctcggcagcacctcagcagcaacatgcccagcaagaagagtggaagaagcggaccccaaccacataaaaggtgggtgttcacgctgaataatccttccgaagacgagcgcaagaaaatacgggagctcccaatctccctatttgattattttattgttggcgaggaaggtaatgaggagggccgaacaccgcacctacaggggttcgctaattttgtgaagaagcaaacttttaataaagtgaagtggtattttggtgcccgctgccacatcgagaaagcgaaaggaacagatcagcagaataaagaatattgcagtaaagaaggcaacttactgatagaatgtggagctcctagatctcaaggacaacggagtgacctctctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagacgaatgtacacgtcattgtggggccacctgggtgtggcaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactctgtgatcgatatcctttgactgttgagactaaaggtggaactgtaccttttttggcccgcagtattctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactga

SEQ ID NO: 23 - DNA, 4667 bp, synthetic construct
  source 1..4667: mol_type = other DNA; note = nucleotide sequence
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacctcgcgaatgcatctagatggcctttttggccactcagtaatttattttatatgggaaaagggcacagggtgggtccactgcttcaaatcggccttcgggtacctccgtggattgttctccagcagtcttccaaaattgcaaagtagtaatcctccgatagagagcttctacagctgggacagcagttgaggagtaccattcctggggggcctgattgctggtaatcaaaatactgcgggccaaaaaaggaacagtaccccctttagtctctacagtcaatggataccggtcacacagtctcagtagatcatcccaaggtaaccagccataaaaatcatccaaaacaacaacttcttctccatgatatccatcccaccacttatttctactaggcttccagtaggtgtcgctaggctcagcaaaattacgggcccactggctcttcccacaaccgggcgggcccactatgacgtgtacagctgtcttccaatcacgctgctgcatcttcccgctcactttcaaaagttcagccagcccgcggaaatttctcacatacgttacagggaactgctcggctacagtcaccaaagaccccgtctccaaaagggtactcacagcagtagacaggtcgctgcgcttcccctggttccgcggagctccacactcgataagtatgtggccttctttactgcagtattctttattctgctggtcggttcctttcgctttctcgatgtggcagcgggcaccaaaataccacttcaccttgttaaaagtctgcttcttagcaaaattcgcaaacccctggaggtgaggagttctaccctcttccaaaccttcctctccgcaaacaaaataatcaaaaagggagattggaagctcccgtattttgtttttctcctcctcggaaggattattaagggtgaacacccacctcttatggggttgcgggccgcttttcctgcttggcattttcactgacgctgccgaggtgctgccgctgccgaagtgcgctggtaatactacagcagcgcacttctttcacttttataggatgacgtatccaaggaggcgtttccgcagacgaagacaccgcccccgcagccatcttggccagatcctccgccgccgcccctggctcgtccacccccgccaccgttaccgctggagaaggaaaaatggcatcttcaacacccgcctctcccgcaccatcggttatactgtcaagaaaaccacagtcagaacgccctcctggaatgtggacatgatgagatttaatattaatgattttcttcccccaggagggggctcaaaccccctcactgtgccctttgaatactacagaataaggaaggttaaggttgaattctggccctgctccccaatcacccagggtgacaggggagtgggctccactgctgttattctagatgataactttgtaacaaaggccaatgccctaacctatgacccctatgtaaactactcctcccgccataccataacccagcccttctcctaccactcccggtactttaccccgaaacctgtccttgataggacaatcgattacttccaacccaataacaaaagaaatcaactctggctgagactacaaactactggaaatgtagaccatgtaggcctcggcactgcgttcgaaaacagtatatacgaccaggactacaatatccgtataaccatgtatgtacaattcagagaatttaatcttaaagaccccccacttaaccctaagtaaaaataaaaaccattacgatgtgataacaaaaaagactcagtaatttattttatatgggaaaagggcacagggtgggtccactgcttcaaatcggccttcgggtacctccgtggattgttctccagcagtcttccaaaattgcaaagtagtaatcctccgatagagagcttctacagctgggacagcagttgaggagtaccattcctggggggcctgattgctggtaatcaaaatactgcgggccaaaaaaggaacagtaccccctttagtctctacagtcaatggataccggtcacacagtctcagtagatcatcccaaggtaaccagccataaggcctttttggccatcggatcccgggcccgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 24 - DNA, 1767 bp, synthetic construct
  source 1..1767: mol_type = other DNA; note = nucleotide sequence of cPCV1-2a
accagcgcacttcggcagcggcagcacctcggcagcgtcagtgaaaatgccaagcaagaaaagcggcccgcaaccccataagaggtgggtgttcacccttaataatccttccgaggaggagaaaaacaaaatacgggagcttccaatctccctttttgattattttgtttgtggcgaggaaggtttggaagagggtagaactcctcacctccaggggtttgcgaattttgctaagaagcagacttttaacaaggtgaagtggtattttggtgcccgctgccacatcgagaaagcgaaaggaaccgaccagcagaataaagaatactgcagtaaagaaggccacatacttatcgagtgtggagctccgcggaaccaggggaagcgcagcgacctgtctactgctgtgagtacccttttggagacggggtctttggtgactgtagccgagcagtttcctgtaacgtatgtgagaaatttccgcgggctggctgaacttttgaaagtgagcgggaagatgcagcagcgtgattggaagacagctgtacacgtcatagtgggcccgcccggttgtgggaagagccagtgggcccgtaattttgctgagcctagggacacctactggaagcctagtagaaataagtggtgggatggatatcatggagaagaagttgttgttttggatgatttttatggctggttaccttgggatgatctactgagactgtgtgaccggtatccattgactgtagagactaaagggggtactgttccttttttggcccgcagtattttgattaccagcaatcaggccccccaggaatggtactcctcaactgctgtcccagctgtagaagctctctatcggaggattactactttgcaattttggaagactgctggagaacaatccacggaggtacccgaaggccgatttgaagcagtggacccaccctgtgcccttttcccatataaaataaattactgagtcttttttgttatcacatcgtaatggtttttatttttattcatttagggtttaagtggggggtctttaagattaaattctctgaattgtacatacatggttacacggatattgtagtcctggtcgtatatactgttttcgaacgcagtgccgaggcctacgtggtccacatttctagaggtttgtagcctcatccaaagctgattccttttgttatttggttggaagtaatcaatggtggagtcaagaacaggtttgggtgtgaagtaacgggagtggtaggagaagggttgggggattgtatggcgggaggagtagtttacatatgggtcataggttagggctgtggcctttgttacaaagttatcatctagaataacagcagtggagcccactcccctatcaccctgggtgatgggggagcagggccagaattcaaccttaacctttcttattctgtagtattcaaagggtatagagattttgttggtcccccctcccgggggaacaaagtcgtcaatattaaatctcatcatgtccaccgcccaggagggcgttctgactgtggtagccttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttctgcggtaacgcctccttggatacgtcatcctataaaagtgaaagaagtgcgctgctgtagtatt

SEQ ID NO: 25 - DNA, 1759 bp, synthetic construct
  source 1..1759: mol_type = other DNA; note = nucleotide sequence for cPCV1-2b
cttttttgttatcacatcgtaatggtttttatttttaagggttaagtggggggtctttaagattaaattctctgaattgtacatacatggttacacggatattgtattcctggtcgtatatactgttttcgaacgcagtgccgaggcctacgtggtctacatttccagcagtttgtagtctcagccacagctggtttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttctgactgtggttcgcttgatagtatatccgaaggtgcgggataggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatcctataaaagtgaaagaagtgcgctgctgtagtattaccagcgcacttcggcagcggcagcacctcggcagcgtcagtgaaaatgccaagcaggaaaagcggcccgcaaccccataagaggtgggtgttcacccttaataatccttccgaggaggagaaaaacaaaatacgggagcttccaatctccctttttgattattttgtttgcggagaggaaggtttggaagagggtagaactcctcacctccaggggtttgcgaattttgctaagaagcagacttttaacaaggtgaagtggtattttggtgcccgctgccacatcgagaaagcgaaaggaaccgaccagcagaataaagaatactgcagtaaagaaggccacatacttatcgagtgtggagctccgcggaaccaggggaagcgcagcgacctgtctactgctgtgagtacccttttggagacggggtctttggtgactgtagccgagcagttccctgtaacgtatgtgagaaatttccgcgggctggctgaacttttgaaagtgagcgggaagatgcagcagcgtgattggaagacagctgtacacgtcatagtgggcccgcccggttgtgggaagagccagtgggcccgtaattttgctgagcctagcgacacctactggaagcctagtagaaataagtggtgggatggatatcatggagaagaagttgttgttttggatgatttttatggctggttaccttgggatgatctactgagactgtgtgaccggtatccattgactgtagagactaaagggggtactgttccttttttggcccgcagtattttgattaccagcaatcaggccccccaggaatggtactcctcaactgctgtcccagctgtagaagctctctatcggaggattactactttgcaattttggaagactgctggagaacaatccacggaggtacccgaaggccgatttgaagcagtggacccaccctgtgcccttttcccatataaaataaattactgagt

SEQ ID NO: 26 - DNA, 20 bp, synthetic construct
  source 1..20: mol_type = other DNA; note = forward primer used to PCR amplify Influenza A Virus
atctattcaacggtcgccag

SEQ ID NO: 27 - DNA, 20 bp, synthetic construct
  source 1..20: mol_type = other DNA; note = reverse primer used to PCR amplify Influenza A Virus
ggatgtgctctaatgggtcg

SEQ ID NO: 28 - DNA, 24 bp, synthetic construct
  source 1..24: mol_type = other DNA; note = PCR amplification of Influenza A Virus
ttggtactggtagtctccctgggg

SEQ ID NO: 29 - DNA, 17 bp, synthetic construct
  source 1..17: mol_type = other DNA; note = forward primer used to PCR amplify BVDV
catgcccatagtaggac

SEQ ID NO: 30 - DNA, 17 bp, synthetic construct
  source 1..17: mol_type = other DNA; note = reverse primer used to PCR amplify BVDV
ccatgtgccatgtacag

SEQ ID NO: 31 - DNA, 20 bp, synthetic construct
  source 1..20: mol_type = other DNA; note = Forward primer used to PCR amplify the Porcine Epidemic Diarrhea Virus
gaattcccaagggcgaaaat

SEQ ID NO: 32 - DNA, 21 bp, synthetic construct
  source 1..21: mol_type = other DNA; note = Reverse primer used to PCR amplify the Porcine Epidemic Diarrhea Virus
ttttcgacaaattccgcatct

SEQ ID NO: 33 - DNA, 23 bp, synthetic construct
  source 1..23: mol_type = other DNA; note = successful PCR amplification of Porcine Epidemic Diarrhea Virus
cgtagcagcttgcttcggaccca